(19) United States
(12) Patent Application Publication
(10) Pub. No.: US 2022/0170033 A1
(43) Pub. Date: Jun. 2, 2022

(54) PLANT EXPLANT TRANSFORMATION

(71) Applicants: PIONEER HI-BRED INTERNATIONAL, INC., JOHNSTON, IA (US); CORTEVA AGRISCIENCE LLC, INDIANAPOLIS, IN (US)

(72) Inventors: MAREN L ARLING, DES MOINES, IA (US); SHUJUN CHANG, JOHNSTON, IA (US); WILLIAM JAMES GORDON-KAMM, URBANDALE, IA (US); NATHALIE SANYOUR-DOYEL, POLK CITY, IA (US); NAGESH SARDESAI, JOHNSTON, IA (US)

(73) Assignees: PIONEER HI-BRED INTERNATIONAL, INC., JOHNSTON, IA (US); CORTEVA AGRISCIENCE LLC, INDIANAPOLIS, IN (US)

(21) Appl. No.: 17/442,552

(22) PCT Filed: Mar. 26, 2020
(86) PCT No.: PCT/US2020/024814

§ 371 Date: Sep. 23, 2021

Related U.S. Application Data
(60) Provisional application No. 62/824,746, filed on Mar. 27, 2019.

(51) Int. Cl. C12N 15/82; C07K 14/415; C12N 9/22

(52) U.S. Cl. CPC C12N 15/8213; C07K 14/415; C12N 2310/20; C12N 15/8241; C12N 15/8205; C12N 9/22

(57) ABSTRACT

Methods for transforming dicot vegetative plant organs and their composite tissues are provided.

### CROSS-REFERENCE TO RELATED APPLICATIONS

This application claims the benefit of PCT Application Serial Number PCT/US2020/024814, filed Mar. 26, 2020, which claims the benefit of U.S. Provisional Application No. 62/824,746, filed Mar. 27, 2019, all of which are hereby incorporated herein by reference in their entireties.

### FIELD OF THE DISCLOSURE

The present disclosure relates to the field of plant molecular biology, more particularly to vegetative plant organs and their composite tissues transformation in dicot plants.

### REFERENCE TO SEQUENCE LISTING SUBMITTED ELECTRONICALLY

The official copy of the sequence listing is submitted electronically via EFS-Web as an ASCII formatted sequence listing with a file named 8045-US-PCT Seq TXT created on Jul. 14, 2021, and having a size of 1,151,693 bytes and is filed concurrently with the specification. The sequence listing contained in this ASCII formatted document is part of the specification and is herein incorporated by reference in its entirety.

### BACKGROUND OF THE DISCLOSURE

In recent years, there has been a tremendous expansion of the capabilities for the genetic engineering of plants. Current transformation technology provides an opportunity to produce commercially viable transgenic plants, enabling the creation of new plant varieties containing desirable traits. One limitation of the genetic engineering of plants is the availability of plant tissue explants that are amenable to transformation since many plant tissue explants are recalcitrant to transformation and regeneration. Thus, there is a need for plant transformation methods permitting a broader range of transformable and regenerable plant explant tissues.

### SUMMARY OF THE DISCLOSURE

The present disclosure comprises methods and compositions for producing transgenic plants that contain a heterologous polynucleotide and methods and compositions for producing gene edited plants. In a further aspect, the present disclosure provides a seed from the plant produced by the methods disclosed herein.

The present disclosure provides a method of producing a transgenic dicot plant that contains a heterologous polynucleotide comprising contacting a dicot vegetative plant organ or its composite tissue with a T-DNA containing the heterologous polynucleotide and a morphogenic gene expression cassette; selecting a plant cell containing the heterologous polynucleotide and no morphogenic gene expression cassette, wherein the plant cell forms a regenerable plant structure containing the heterologous polynucleotide and no morphogenic gene expression cassette; and regenerating a transgenic plant from the regenerable plant structure containing the heterologous polynucleotide and no morphogenic gene expression cassette. In a further aspect, the morphogenic gene expression cassette comprises (i) a nucleotide sequence encoding a functional WUS/WOX polypeptide; or (ii) a nucleotide sequence encoding a Babyboom (BBM) polypeptide or an Ovule Development Protein 2 (ODP2) polypeptide; or (iii) a combination of (i) and (ii). In a further aspect, the nucleotide sequence encodes the functional WUS/WOX polypeptide. In a further aspect, the nucleotide sequence encoding the functional WUS/WOX polypeptide is selected from WUS, WUS1, WUS2, WUS3, WOX2 A, WOX4, WOX5, and WOX9. In a further aspect, the nucleotide sequence encodes the Babyboom (BBM) polypeptide or the Ovule Development Protein 2 (ODP2) polypeptide. In a further aspect, the nucleotide sequence encoding the Babyboom (BBM) polypeptide is selected from BBM2, BMN2, and BMN3 or the Ovule Development Protein 2 (ODP2) polypeptide is ODP2. In a further aspect, the nucleotide sequence encodes the functional WUS/WOX polypeptide and the Babyboom (BBM) polypeptide or the Ovule Development Protein 2 (ODP2) polypeptide. In a further aspect, the nucleotide sequence encoding the functional WUS/WOX polypeptide is selected from WUS, WUS1, WUS2, WUS3, WOX2 A, WOX4, WOX5, and WOX9 and the Babyboom (BBM) polypeptide is selected from BBM2, BMN2, and BMN3 or the Ovule Development Protein 2 (ODP2) polypeptide is ODP2. In a further aspect, the heterologous polynucleotide is selected from the group consisting of a heterologous polynucleotide conferring a nutritional enhancement, a heterologous polynucleotide conferring a modified oil content, a heterologous polynucleotide conferring a modified protein content, a heterologous polynucleotide conferring a modified metabolite content, a heterologous polynucleotide conferring increased yield, a heterologous polynucleotide conferring abiotic stress tolerance, a heterologous polynucleotide conferring drought tolerance, a heterologous polynucleotide conferring cold tolerance, a heterologous polynucleotide conferring herbicide tolerance, a heterologous polynucleotide conferring pest resistance, a heterologous polynucleotide conferring pathogen resistance, a heterologous polynucleotide conferring insect resistance, a heterologous polynucleotide conferring nitrogen use efficiency (NUE), a heterologous polynucleotide conferring disease resistance, a heterologous polynucleotide conferring increased biomass, a heterologous polynucleotide conferring an ability to alter a metabolic pathway, and a combination of the foregoing. In a further aspect, the dicot vegetative plant organ or its composite tissue is selected from the group consisting of a leaf explant, a leaf primordia, a stipule, a cotyledon, a cotyledonary node, a mesocotyl, a stem explant, a primary root, a lateral secondary root, a root segment, a bud, and a meristem, including but not limited to an apical meristem, a root meristem, a secondary meristem, an axillary meristem, a floral meristem, and a combination of the foregoing. In a further aspect, the leaf explant is selected from the group consisting of a leaf, a radical leaf, a cauline leaf, an alternate leaf, and opposite leaf, a decussate leaf, an opposite superposed leaf, a whorled leaf, a petiolate leaf, a sessile leaf, a subsessile leaf, a stipulate leaf, an exstipulate leaf, a simple leaf, a compound leaf, and a combination of the foregoing. In a further aspect, the stem explant is selected from the group consisting of a stem nodal region, a stem internodal region, a petiole, a hypocotyl, an epicotyl, a stolon, a rhizome, a tuber, a corm, and a combination of the foregoing. In a further aspect, the dicot is selected from the group consisting of soybean, cotton, sunflower, cassava, common bean, cowpea, tomato, potato, beet, grape, Eucalyptus, citrus, papaya, cacao, cucumber, apple, Capsicum, melon, and Brassica. In a further aspect, the morphogenic gene expression cassette comprises a polynucleotide encoding a functional WUS/WOX polypeptide, wherein the functional WUS/WOX polypeptide comprises an amino acid sequence of any of SEQ ID NOS: 61, 63, 65, 67, 69, 71, 73, 75, 77, 79, 81, 83, 85, 87, 89, 91, 93, 95, 97, 99, 101, 103, 105, 107, 128, 130, 132, 134, 136, 138, 140, 142, 144, 146, or 148; or wherein the functional WUS/WOX polypeptide is encoded by a nucleotide sequence of any of SEQ ID NOS: 60, 62, 64, 66, 68, 70, 72, 74, 76, 78, 80, 82, 84, 86, 88, 90, 92, 94, 96, 98, 100, 102, 104, 106, 127, 129, 131, 133, 135, 137, 139, 141, 143, 145, or 147. In a further aspect, the morphogenic gene expression cassette further comprises a polynucleotide sequence encoding a site-specific recombinase selected from the group consisting of FLP, FLPe, KD, Cre, SSV1, lambda Int, phi C31 Int, HK022, R, B2, B3, Gin, Tn1721, CinH, ParA, Tn5053, Bxb1, TP907-1, or U153, wherein the site-specific recombinase is operably linked to a constitutive promoter, an inducible promoter, a tissue-specific promoter, or a developmentally regulated promoter. In a further aspect, the method further comprising excising the morphogenic gene expression cassette. In a further aspect, a transgenic plant produced by the method is provided. In a further aspect, a seed of the transgenic plant produced by the method is provided, wherein the seed comprises the heterologous polynucleotide. In a further aspect, the regenerable plant structure is formed at an increased frequency of from about 0.1% to about 1.0%, from about 1.1% to about 10%, from about 10.1% to about 20%, from about 20.1% to about 30%, from about 30.1% to about 40%, from about 40.1% to about 50%, from about 50.1% to about 60%, from about 60.1% to about 70%, from about 70.1% to about 80%, from about 80.1% to about 90%, and from about 90.1% to about 100%, compared to the frequency of regenerable plant structures formed when the dicot vegetative plant organ or its composite tissue is not contacted with the morphogenic gene expression cassette.

The present disclosure provides a method of producing a genome-edited dicot plant comprising contacting a dicot vegetative plant organ or its composite tissue with a T-DNA containing a morphogenic gene expression cassette and providing a polynucleotide encoding a site-specific polypeptide or a site-specific polypeptide; selecting a plant cell containing a genome edit and no morphogenic gene expression cassette, wherein the plant cell forms a regenerable plant structure containing the genome edit and no morphogenic gene expression cassette; and regenerating a genome-edited plant from the regenerable plant structure containing the genome edit and no morphogenic gene expression cassette. In a further aspect, the morphogenic gene expression cassette comprises (i) a nucleotide sequence encoding a functional WUS/WOX polypeptide; or (ii) a nucleotide sequence encoding a Babyboom (BBM) polypeptide or an Ovule Development Protein 2 (ODP2) polypeptide; or (iii) a combination of (i) and (ii). In a further aspect, the nucleotide sequence encodes the functional WUS/WOX polypeptide. In a further aspect, the nucleotide sequence encoding the functional WUS/WOX polypeptide is selected from WUS, WUS1, WUS2, WUS3, WOX2 A, WOX4, WOX5, and WOX9. In a further aspect, the nucleotide sequence encodes the Babyboom (BBM) polypeptide or the Ovule Development Protein 2 (ODP2) polypeptide. In a further aspect, the nucleotide sequence encoding the Babyboom (BBM) polypeptide is selected from BBM2, BMN2, and BMN3 or the Ovule Development Protein 2 (ODP2) polypeptide is ODP2. In a further aspect, the nucleotide sequence encodes the functional WUS/WOX polypeptide and the Babyboom (BBM) polypeptide or the Ovule Development Protein 2 (ODP2) polypeptide. In a further aspect, the nucleotide sequence encoding the functional WUS/WOX polypeptide is selected from WUS, WUS1, WUS2, WUS3, WOX2 A, WOX4, WOX5, and WOX9 and the Babyboom (BBM) polypeptide is selected from BBM2, BMN2, and BMN3 or the Ovule Development Protein 2 (ODP2) polypeptide is ODP2. In a further aspect, the site-specific polypeptide is selected from the group consisting of a zinc finger nuclease, a meganuclease, TALEN, and a CRISPR-Cas nuclease. In a further aspect, wherein the CRISPR-Cas nuclease is Cas9 or Cpfl nuclease and further comprising providing a guide RNA. In a further aspect, the site-specific nuclease effects an insertion, a deletion, or a substitution mutation. In a further aspect, the guide RNA and CRISPR-Cas nuclease is a ribonucleoprotein complex. In a further aspect, the dicot vegetative plant organ or its composite tissue is selected from the group consisting of a leaf explant, a leaf primordia, a stipule, a cotyledon, a cotyledonary node, a mesocotyl, a stem explant, a primary root, a lateral secondary root, a root segment, a bud, and a meristem, including but not limited to an apical meristem, a root meristem, a secondary meristem, an axillary meristem, a floral meristem, and a combination of the foregoing. In a further aspect, the leaf explant is selected from the group consisting of a leaf, a radical leaf, a cauline leaf, an alternate leaf, and opposite leaf, a decussate leaf, an opposite superposed leaf, a whorled leaf, a petiolate leaf, a sessile leaf, a subsessile leaf, a stipulate leaf, an exstipulate leaf, a simple leaf, a compound leaf, and a combination of the foregoing. In a further aspect, the stem explant is selected from the group consisting of a stem nodal region, a stem internodal region, a petiole, a hypocotyl, an epicotyl, a stolon, a rhizome, a tuber, a corm, and a combination of the foregoing. In a further aspect, the dicot, is selected from the group consisting of soybean, cotton, sunflower, cassava, common bean, cowpea, tomato, potato, beet, grape, Eucalyptus, citrus, papaya, cacao, cucumber, apple, Capsicum, melon, and Brassica. In a further aspect, the morphogenic gene expression cassette comprises a polynucleotide encoding a functional WUS/WOX polypeptide, wherein the functional WUS/WOX polypeptide comprises an amino acid sequence of any of SEQ ID NOS: 61, 63, 65, 67, 69, 71, 73, 75, 77, 79, 81, 83, 85, 87, 89, 91, 93, 95, 97, 99, 101, 103, 105, 107, 128, 130, 132, 134, 136, 138, 140, 142, 144, 146, or 148; or wherein the functional WUS/WOX polypeptide is encoded by a nucleotide sequence of any of SEQ ID NOS: 60, 62, 64, 66, 68, 70, 72, 74, 76, 78, 80, 82, 84, 86, 88, 90, 92, 94, 96, 98, 100, 102, 104, 106, 127, 129, 131, 133, 135, 137, 139, 141, 143, 145, or 147. Ina further aspect, the morphogenic gene expression cassette further comprises a polynucleotide sequence encoding a site-specific recombinase selected from the group consisting of FLP, FLPe, KD, Cre, SSV1, lambda Int, phi C31 Int, HK022, R, B2, B3, Gin, Tn1721, CinH, ParA, Tn5053, Bxb1, TP907-1, or U153, wherein the site-specific recombinase is operably linked to a constitutive promoter, an inducible promoter, a tissue-specific promoter, or a developmentally regulated promoter. In a further aspect, further comprising excising the morphogenic gene expression cassette. In a further aspect, the genome-edited plant produced by the method is provided. In a further aspect, a seed of the genome-edited plant produced by the method is provided, wherein the seed comprises the genome edit. In a further aspect, the regenerable plant structure is formed at an increased frequency of from about 0.1% to about 1.0%, from about 1.1% to about 10%, from about 10.1% to about 20%, from about 20.1% to about 30%, from about 30.1% to about 40%, from about 40.1% to about 50%, from about 50.1% to about 60%, from about 60.1% to about 70%, from about 70.1% to about 80%, from about 80.1% to about 90%, and from about 90.1% to about 100%, compared to the frequency of genome-edited regenerable plant structures formed when the dicot vegetative plant organ or its composite tissue is not contacted with the morphogenic gene expression cassette.

### BRIEF DESCRIPTION OF THE FIGURES

FIG. 1 shows a graphical representation of the percent (%) of tobacco leaf segments with de novo shoots after Agrobacterium infection with the different WUS genes as described in Example 14.

### DETAILED DESCRIPTION

The disclosures herein will be described more fully hereinafter with reference to the accompanying FIGURE, in which some, but not all possible aspects are shown. Indeed, disclosures may be embodied in many different forms and should not be construed as limited to the aspects set forth herein; rather, these aspects are provided so that this disclosure will satisfy applicable legal requirements.

Many modifications and other aspects disclosed herein will come to mind to one skilled in the art to which the disclosed methods pertain having the benefit of the teachings presented in the following descriptions and the associated FIGURE. Therefore, it is to be understood that the disclosures are not to be limited to the specific aspects disclosed and that modifications and other aspects are intended to be included within the scope of the appended claims. Although specific terms are employed herein, they are used in a generic and descriptive sense only and not for purposes of limitation.

It is also to be understood that the terminology used herein is for the purpose of describing particular aspects only and is not intended to be limiting. As used in the specification and in the claims, the term “comprising” can include the aspect of “consisting of” Unless defined otherwise, all technical and scientific terms used herein have the same meaning as commonly understood by one of ordinary skill in the art to which the disclosed compositions and methods belong. In this specification and in the claims which follow, reference will be made to a number of terms which shall be defined herein.

As used herein, a “regenerable plant structure” is a multicellular structure capable of forming a fully functional fertile plant. Regenerable plant structures capable of forming a fully functional fertile plant include but are not limited to, shoot meristem, shoots, somatic embryos, embryogenic callus, somatic meristems, and/or organogenic callus.

As used herein, a “somatic embryo” is a multicellular structure that progresses through developmental stages that are similar to the development of a zygotic embryo, including formation of globular and transition-stage embryos, formation of an embryo axis and a scutellum, and accumulation of lipids and starch. Single somatic embryos derived from a zygotic embryo germinate to produce single non-chimeric plants, which may originally derive from a single-cell.

As used herein, an “embryogenic callus” is a friable or non-friable mixture of undifferentiated or partially undifferentiated cells which subtend proliferating primary and secondary somatic embryos capable of regenerating into mature fertile plants.

As used herein, a “somatic meristem” is a multicellular structure that is similar to the apical meristem which is part of a seed-derived embryo, characterized as having an undifferentiated apical dome flanked by leaf primordia and subtended by vascular initials, the apical dome giving rise to an above-ground vegetative plant. Such somatic meristems can form single or fused clusters of meristems.

As used herein, an “organogenic callus” is a compact mixture of differentiated growing plant structures, including, but not limited to, apical meristems, root meristems, leaves and roots.

As used herein, “germination” is the growth of a regenerable structure to form a plantlet which continues growing to produce a plant.

As used herein, a “transgenic plant” is a mature, fertile plant that contains a transgene.

The methods of the disclosure can be used to transform vegetative plant organs and their composite tissues. As used herein, “vegetative plant organs and their composite tissues” include but are not limited to leaf explants, leaf primordia, stipule, cotyledons, cotyledonary nodes, mesocotyl, stem explants, primary roots, lateral secondary roots, root segments, buds, and meristems, including but not limited to apical meristems, root meristems, secondary meristems, axillary meristems, and floral meristems. As used herein, “stem explants” include but are not limited to the nodal and internodal regions of the stem, the petiole, hypocotyl, epicotyl, stolon, rhizome, tuber, and corm. As used herein, “leaf explants” include but are not limited to radical leaves, cauline leaves, alternate leaves, opposite leaves, decussate leaves, opposite superposed leaves, whorled leaves, petiolate leaves, sessile leaves, subsessile leaves, stipulate leaves, exstipulate leaves, simple leaves, or compound leaves. Leaf explants include the leaf base or the portion of the leaf immediately proximal to its attachment point to the petiole or stem. Such vegetative organs and their composite tissues can be used for transformation with nucleotide sequences encoding agronomically important traits.

As used herein, a “leaf” is a flat lateral structure that protrudes from a plant's stem, including the supporting stalk between the flattened leaf and the plant stem, but not including the axillary meristem located at the junction of the petiole and stem, including but not limited to a radical leaf, a cauline leaf, an alternate leaf, and opposite leaf, a decussate leaf, an opposite superposed leaf, a whorled leaf, a petiolate leaf, a sessile leaf, a subsessile leaf, a stipulate leaf, an exstipulate leaf, a simple leaf, or a compound leaf.

As used herein, a “stem internode” is the tissue located in the intervals between stem nodes of the plant, with the “stem node” being the region of the stem from which branches, petioles, leaves, or aerial roots grow out of the stem.

As used herein, the term “morphogenic gene” means a gene that when ectopically expressed stimulates formation of a somatically-derived structure that can produce a plant. More precisely, ectopic expression of the morphogenic gene stimulates the de novo formation of a somatic embryo or an organogenic structure, such as a shoot meristem, that can produce a plant. This stimulated de novo formation occurs either in the cell in which the morphogenic gene is expressed, or in a neighboring cell. A morphogenic gene can be a transcription factor that regulates expression of other genes, or a gene that influences hormone levels in a plant tissue, both of which can stimulate morphogenic changes. A morphogenic gene may be stably incorporated into the genome of a plant or it may be transiently expressed. In an aspect, expression of the morphogenic gene is controlled. The controlled expression may be a pulsed expression of the morphogenic gene for a particular period of time. Alternatively, the morphogenic gene may be expressed in only some transformed cells and not expressed in others. The control of expression of the morphogenic gene can be achieved by a variety of methods as disclosed herein below. The morphogenic genes useful in the methods of the present disclosure may be obtained from or derived from any plant species described herein.

As used herein, the term “morphogenic factor” means a morphogenic gene and/or the protein expressed by a morphogenic gene.

A morphogenic gene is involved in plant metabolism, organ development, stem cell development, cell growth stimulation, organogenesis, regeneration, somatic embryogenesis initiation, accelerated somatic embryo maturation, initiation and/or development of the apical meri stem, initiation and/or development of shoot meristem, initiation and/or development of shoots, or a combination thereof, such as WUS/WOX genes (WUS, WUS1, WUS2, WUS3, WOX2 A, WOX4, WOX5, or WOX9) see U.S. Pat. Nos. 7,348,468 and 7,256,322 and United States Patent Application publications 20170121722 and 20070271628; Laux et al. (1996) Development 122:87-96; and Mayer et al. (1998) Cell 95:805-815; van der Graaff et al., 2009, Genome Biology 10:248; Dolzblasz et al., 2016, Mol. Plant 19:1028-39 are useful in the methods of the disclosure. Modulation of WUS/WOX is expected to modulate plant and/or plant tissue phenotype including plant metabolism, organ development, stem cell development, cell growth stimulation, organogenesis, regeneration, somatic embryogenesis initiation, accelerated somatic embryo maturation, initiation and/or development of the apical meristem, initiation and/or development of shoot meristem, initiation and/or development of shoots, or a combination thereof. Expression of Arabidopsis WUS can induce stem cells in vegetative tissues, which can differentiate into somatic embryos (Zuo, et al. (2002) Plant J 30:349-359). Also of interest in this regard would be a MYB118 gene (see U.S. Pat. No. 7,148,402), MYB115 gene (see Wang et al. (2008) Cell Research 224-235), a BABYBOOM gene (BBM; see Boutilier et al. (2002) Plant Cell 14:1737-1749), or a CLAVATA gene (see, for example, U.S. Pat. No. 7,179,963).

Morphogenic polynucleotide sequences and amino acid sequences of functional WUS/WOX polypeptides are useful in the disclosed methods. As defined herein, a “functional WUS/WOX nucleotide” is any polynucleotide encoding a protein that contains a homeobox DNA binding domain, a WUS box, and an EAR repressor domain (Ikeda et al., 2009 Plant Cell 21:3493-3505). As demonstrated by Rodriguez et al., 2016 PNAS www.pnas.org/cgi/doi/10.1073/pnas.1607673113 removal of the dimerization sequence which leaves behind the homeobox DNA binding domain, a WUS box, and an EAR repressor domain results in a functional WUS/WOX polypeptide. The Wuschel protein, designated hereafter as WUS, plays a key role in the initiation and maintenance of the apical meristem, which contains a pool of pluripotent stem cells (Endrizzi, et al., (1996) Plant Journal 10:967-979; Laux, et al., (1996) Development 122:87-96; and Mayer, et al., (1998) Cell 95:805-815). Arabidopsis plants mutant for the WUS gene contain stem cells that are misspecified and that appear to undergo differentiation. WUS encodes a novel homeodomain protein which presumably functions as a transcriptional regulator (Mayer, et al., (1998) Cell 95:805-815). The stem cell population of Arabidopsis shoot meristems is believed to be maintained by a regulatory loop between the CLAVATA (CLV) genes which promote organ initiation and the WUS gene which is required for stem cell identity, with the CLV genes repressing WUS at the transcript level, and WUS expression being sufficient to induce meristem cell identity and the expression of the stem cell marker CLV3 (Brand, et al., (2000) Science 289:617-619; Schoof, et al., (2000) Cell 100:635-644). Constitutive expression of WUS in Arabidopsis has been shown to lead to adventitious shoot proliferation from leaves (in planta) (Laux, T., Talk Presented at the XVI International Botanical Congress Meeting, Aug. 1-7, 1999, St. Louis, Mo.).

In an aspect, the functional WUS/WOX polypeptides useful in the methods of the present disclosure is a WUS, WUS1, WUS2, WUS3, WOX2 A, WOX4, WOX5, WOX5 A, or WOX9 polypeptide (see, U.S. Pat. Nos. 7,348,468 and 7,256,322 and US Patent Application Publication Numbers 2017/0121722 and 2007/0271628, herein incorporated by reference in their entirety and van der Graaff et al., 2009, Genome Biology 10:248). The functional WUS/WOX polypeptides useful in the methods of the present disclosure can be obtained from or derived from any plant including but not limited to monocots, dicots, Angiospermae, and Gymnospermae. Additional WUS/WOX genes useful in the methods of the present disclosure are listed in Table 3.

Other morphogenic genes useful in the present disclosure include, but are not limited to, LEC1 (U.S. Pat. No. 6,825,397 incorporated herein by reference in its entirety, Lotan et al., 1998, Cell 93:1195-1205), LEC2 (Stone et al., 2008, PNAS 105:3151-3156; Belide et al., 2013, Plant Cell Tiss. Organ Cult 113:543-553), KN1/STM (Sinha et al., 1993. Genes Dev 7:787-795), the IPT gene from Agrobacterium (Ebinuma and Komamine, 2001, In vitro Cell. Dev Biol-Plant 37:103-113), MONOPTEROS-DELTA (Ckurshumova et al., 2014, New Phytol. 204:556-566), the Agrobacterium AV-6b gene (Wabiko and Minemura 1996, Plant Physiol. 112:939-951), the combination of the Agrobacterium IAA-h and IAA-m genes (Endo et al., 2002, Plant Cell Rep., 20:923-928), the Arabidopsis SERK gene (Hecht et al., 2001, Plant Physiol. 127:803-816), the Arabidopsis AGL15 gene (Harding et al., 2003, Plant Physiol. 133:653-663), the FUSCA gene (Castle and Meinke, Plant Cell 6:25-41), and the PICKLE gene (Ogas et al., 1999, PNAS 96:13839-13844).

As used herein, the term “transcription factor” means a protein that controls the rate of transcription of specific genes by binding to the DNA sequence of the promoter and either up-regulating or down-regulating expression. Examples of transcription factors that are also morphogenic genes, include members of the AP2/EREBP family (including BBM (ODP2)), plethora and aintegumenta sub-families, CAAT-box binding proteins such as LEC1 and HAP3, and members of the MYB, bHLH, NAC, MADS, bZIP and WRKY families.

Morphogenic polynucleotide sequences and amino acid sequences of Ovule Development Protein 2 (ODP2) polypeptides, and related polypeptides, e.g., Babyboom (BBM) protein family proteins are useful in the methods of the disclosure. In an aspect, a polypeptide comprising two AP2-DNA binding domains is an ODP2, BBM2, BMN2, or BMN3 polypeptide see, US Patent Application Publication Number 2017/0121722, herein incorporated by reference in its entirety. ODP2 polypeptides useful in the methods of the disclosure contain two predicted APETALA2 (AP2) domains and are members of the AP2 protein family (PFAM Accession PF00847). The AP2 family of putative transcription factors has been shown to regulate a wide range of developmental processes, and the family members are characterized by the presence of an AP2 DNA binding domain. This conserved core is predicted to form an amphipathic alpha helix that binds DNA. The AP2 domain was first identified in APETALA2, an Arabidopsis protein that regulates meristem identity, floral organ specification, seed coat development, and floral homeotic gene expression. The AP2 domain has now been found in a variety of proteins.

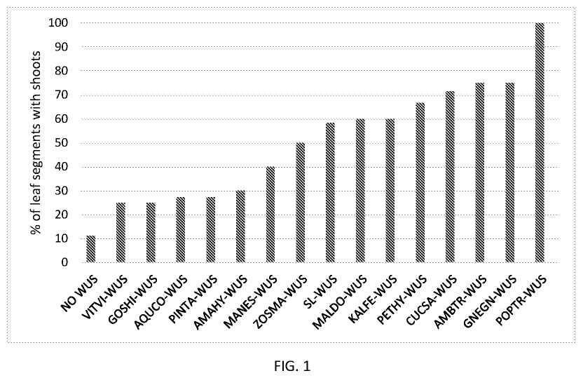

ODP2 polypeptides useful in the methods of the disclosure share homology with several polypeptides within the AP2 family, e.g., see FIG. 1 of U.S. Pat. No. 8,420,893, which is incorporated herein by reference in its entirety, and provides an alignment of the maize and rice ODP2 polypeptides with eight other proteins having two AP2 domains. A consensus sequence of all proteins appearing in the alignment of U.S. Pat. No. 8,420,893 is also provided in FIG. 1 therein. The polypeptide comprising the two AP2-DNA binding domains useful in the methods of the disclosure can be obtained from or derived from any of the plants described herein. In an aspect, the polypeptide comprising the two AP2-DNA binding domains useful in the methods of the disclosure is an ODP2 polypeptide. In an aspect, the polypeptide comprising the two AP2-DNA binding domains useful in the methods of the disclosure is a BBM2 polypeptide. The ODP2 polypeptide and the BBM2 polypeptide useful in the methods of the disclosure can be obtained from or derived from any plant including but not limited to monocots, dicots, Angiospermae, and Gymnospermae.

As used herein, the term “expression cassette” means a distinct component of vector DNA consisting of coding and non-coding sequences including 5′ and 3′ regulatory sequences that control expression in a transformed/transfected cell.

As used herein, the term “coding sequence” means the portion of DNA sequence bounded by a start and a stop codon that encodes the amino acids of a protein.

As used herein, the term “non-coding sequence” means the portions of a DNA sequence that are transcribed to produce a messenger RNA, but that do not encode the amino acids of a protein, such as 5′ untranslated regions, introns and 3′ untranslated regions. Non-coding sequence can also refer to RNA molecules such as micro-RNAs, interfering RNA or RNA hairpins, that when expressed can down-regulate expression of an endogenous gene or another transgene.

As used herein, the term “regulatory sequence” means a segment of a nucleic acid molecule which is capable of increasing or decreasing the expression of a gene. Regulatory sequences include promoters, terminators, enhancer elements, silencing elements, 5′ UTR and 3′ UTR (untranslated regions).

As used herein, the term “transfer cassette” means a T-DNA comprising an expression cassette or expression cassettes flanked by the right border and the left border.

As used herein, T-DNA means a portion of a Ti plasmid that is inserted into the genome of a host plant cell.

As used herein, the term “selectable marker” means a transgene that when expressed in a transformed/transfected cell confers resistance to selective agents such as antibiotics, herbicides and other compounds toxic to an untransformed/untransfected cell.

As used herein, the term “EAR” means an Ethylene-responsive element binding factor-associated Amphiphilic Repression motif” having general consensus sequences that act as transcriptional repression signals within transcription factors. Addition of an EAR-type repressor element to a DNA-binding protein such as a transcription factor, dCAS9, or LEXA (as examples) confers transcriptional repression function to the fusion protein (Kagale, S., and Rozwadowski, K. 2010. Plant Signaling and Behavior 5:691-694).

In an aspect, the transformation methods of the disclosure use the recombinant expression cassette or construct comprising a nucleotide sequence encoding a functional WUS/WOX polypeptide, or a nucleotide sequence encoding a Babyboom (BBM) polypeptide or an Ovule Development Protein 2 (ODP2) polypeptide, or a combination of a nucleotide sequence encoding a functional WUS/WOX polypeptide and a nucleotide sequence encoding a Babyboom (BBM) polypeptide or an Ovule Development Protein 2 (ODP2) polypeptide. In an aspect, the nucleotide sequence encoding the functional WUS/WOX polypeptide, or the nucleotide sequence encoding a Babyboom (BBM) polypeptide or an Ovule Development Protein 2 (ODP2) polypeptide, or the combination of a nucleotide sequence encoding a functional WUS/WOX polypeptide and a nucleotide sequence encoding a Babyboom (BBM) polypeptide or an Ovule Development Protein 2 (ODP2) polypeptide can be targeted for excision by a site-specific recombinase. Thus, the expression of the nucleotide sequence encoding the functional WUS/WOX polypeptide, or the nucleotide sequence encoding a Babyboom (BBM) polypeptide or an Ovule Development Protein 2 (ODP2) polypeptide, or the combination of a nucleotide sequence encoding a functional WUS/WOX polypeptide and a nucleotide sequence encoding a Babyboom (BBM) polypeptide or an Ovule Development Protein 2 (ODP2) polypeptide can be controlled by excision at a desired time post-transformation. It is understood that when a site-specific recombinase is used to control the expression of the nucleotide sequence encoding the functional WUS/WOX polypeptide, or the nucleotide sequence encoding a Babyboom (BBM) polypeptide or an Ovule Development Protein 2 (ODP2) polypeptide, or the combination of a nucleotide sequence encoding a functional WUS/WOX polypeptide and a nucleotide sequence encoding a Babyboom (BBM) polypeptide or an Ovule Development Protein 2 (ODP2) polypeptide, the expression construct comprises appropriate site-specific excision sites flanking the polynucleotide sequences to be excised, e.g., Cre lox sites if Cre recombinase is utilized. It is not necessary that the site-specific recombinase be co-located on the expression construct comprising the nucleotide sequence encoding the functional WUS/WOX polypeptide, or the nucleotide sequence encoding a Babyboom (BBM) polypeptide or an Ovule Development Protein 2 (ODP2) polypeptide, or the combination of a nucleotide sequence encoding a functional WUS/WOX polypeptide and a nucleotide sequence encoding a Babyboom (BBM) polypeptide or an Ovule Development Protein 2 (ODP2) polypeptide. However, in an aspect, the morphogenic gene expression cassette further comprises a nucleotide sequence encoding a site-specific recombinase.

The site-specific recombinase used to control expression of the nucleotide sequence encoding the functional WUS/WOX polypeptide, or the nucleotide sequence encoding a Babyboom (BBM) polypeptide or an Ovule Development Protein 2 (ODP2) polypeptide, or the combination of a nucleotide sequence encoding a functional WUS/WOX polypeptide and a nucleotide sequence encoding a Babyboom (BBM) polypeptide or an Ovule Development Protein 2 (ODP2) polypeptide can be chosen from a variety of suitable site-specific recombinases. For examples, in various aspects, the site-specific recombinase is FLP, FLPe, KD, Cre, SSV1, lambda Int, phi C31 Int, HK022, R, B2 (Nern et al., (2011) PNAS Vol. 108, No. 34 pp 14198-14203), B3 (Nern et al., (2011) PNAS Vol. 108, No. 34 pp 14198-14203), Gin, Tn1721, CinH, ParA, Tn5053, Bxb1, TP907-1, or U153. The site-specific recombinase can be a destabilized fusion polypeptide. The destabilized fusion polypeptide can be TETR(G17 A)˜CRE or ESR(G17 A)˜CRE.

In an aspect, the nucleotide sequence encoding a site-specific recombinase is operably linked to a constitutive promoter, an inducible promoter, a tissue-specific promoter, or a developmentally-regulated promoter. Suitable constitutive promoters, inducible promoters, tissue-specific promoters, and developmentally-regulated promoters include UBI, LLDAV, EVCV, DMMV, BSV(AY) PRO, CYMV PRO FL, UBIZM PRO, SI-UB3 PRO, SB-UBI PRO (ALT1), USB1ZM PRO, ZM-GOS2 PRO, ZM-H1B PRO (1.2 KB), IN2-2, NOS, the −135 version of 35S, ZM-ADF PRO (ALT2), AXIG1, DRS, XVE, GLB1, OLE, LTP2 (Kalla et al., 1994. Plant J. 6:849-860 and U.S. Pat. No. 5,525,716 incorporated herein by reference in its entirety), HSP17.7, HSP26, HSP18 A, AT-HSP811, AT-HSP811L, GM-HSP173B, promoters activated by tetracycline, ethametsulfuron or chlorsulfuron, PLTP, PLTP1, PLTP2, PLTP3, SDR, LGL, LEA-14 A, or LEA-D34 (United States Patent Application publications 20170121722 and 20180371480 incorporated herein by reference in their entireties).

In an aspect, the chemically inducible promoter operably linked to the site-specific recombinase is XVE. The chemically-inducible promoter can be repressed by the tetracycline repressor (TETR), the ethametsulfuron repressor (ESR), or the chlorsulfuron repressor (CR), and de-repression occurs upon addition of tetracycline-related or sulfonylurea ligands. The repressor can be TETR and the tetracycline-related ligand is doxycycline or anhydrotetracycline. (Gatz, C., Frohberg, C. and Wendenburg, R. (1992) Stringent repression and homogeneous de-repression by tetracycline of a modified CaMV 35S promoter in intact transgenic tobacco plants, Plant J. 2, 397-404). Alternatively, the repressor can be ESR and the sulfonylurea ligand is ethametsulfuron, chlorsulfuron, metsulfuron-methyl, sulfometuron methyl, chlorimuron ethyl, nicosulfuron, primisulfuron, tribenuron, sulfosulfuron, trifloxysulfuron, foramsulfuron, iodosulfuron, prosulfuron, thifensulfuron, rimsulfuron, mesosulfuron, or halosulfuron (US20110287936 incorporated herein by reference in its entirety).

In an aspect, when the morphogenic gene expression cassette or construct comprises site-specific recombinase excision sites, the nucleotide sequence encoding the functional WUS/WOX polypeptide, or the nucleotide sequence encoding a Babyboom (BBM) polypeptide or an Ovule Development Protein 2 (ODP2) polypeptide, or the combination of a nucleotide sequence encoding a functional WUS/WOX polypeptide and a nucleotide sequence encoding a Babyboom (BBM) polypeptide or an Ovule Development Protein 2 (ODP2) polypeptide can be operably linked to an auxin inducible promoter, a developmentally regulated promoter, a tissue-specific promoter, or a constitutive promoter. Exemplary auxin inducible promoters, developmentally regulated promoters, tissue-specific promoters, and constitutive promoters useful in this context include UBI, LLDAV, EVCV, DMMV, BSV(AY) PRO, CYMV PRO FL, UBIZM PRO, SI-UB3 PRO, SB-UBI PRO (ALT1), USB1ZM PRO, ZM-GOS2 PRO, ZM-H1B PRO (1.2 KB), IN2-2, NOS, the −135 version of 35S, ZM-ADF PRO (ALT2), AXIG1 (U.S. Pat. No. 6,838,593 incorporated herein by reference in its entirety), DRS, XVE, GLB1, OLE, LTP2, HSP17.7, HSP26, HSP18 A, AT-HSP811 (Takahashi, T, et al., (1992) Plant Physiol. 99 (2): 383-390), AT-HSP811L (Takahashi, T, et al., (1992) Plant Physiol. 99 (2): 383-390), GM-HSP173B (Schoffl, F., et al. (1984) EMBO J. 3(11): 2491-2497), promoters activated by tetracycline, ethamethsulfuron or chlorsulfuron, PLTP, PLTP1, PLTP2, PLTP3, SDR, LGL, LEA-14 A, LEA-D34 (United States Patent Application publications 20170121722 and 20180371480 incorporated herein by reference in their entireties), and any of the promoters disclosed herein.

When using a morphogenic gene cassette and a trait gene cassette (heterologous polynucleotide) to produce transgenic plants it is desirable to have the ability to segregate the morphogenic gene locus away from the trait gene (heterologous polynucleotide) locus in co-transformed plants to provide transgenic plants containing only the trait gene (heterologous polynucleotide). This can be accomplished using an Agrobacterium tumefaciens two T-DNA binary system, with two variations on this general theme (see Miller et al., 2002). For example, in the first, a two T-DNA vector, where expression cassettes for morphogenic genes and herbicide selection (i.e. HRA) are contained within a first T-DNA and the trait gene cassette (heterologous polynucleotide) is contained within a second T-DNA, where both T-DNA's reside on a single binary vector. When a plant cell is transformed by an Agrobacterium containing the two T-DNA plasmid a high percentage of transformed cells contain both T-DNA's that have integrated into different genomic locations (for example, onto different chromosomes). In the second method, for example, two Agrobacterium strains, each containing one of the two T-DNA's (either the morphogenic gene T-DNA or the trait gene (heterologous polynucleotide) T-DNA), are mixed together in a ratio, and the mixture is used for transformation. After transformation using this mixed Agrobacterium method, it is observed at a high frequency that recovered transgenic events contain both T-DNA's, often at separate genomic locations. For both co-transformation methods, it is observed that in a large proportion of the produced transgenic events, the two T-DNA loci segregate independently and progeny T1 plants can be readily identified in which the T-DNA loci have segregated away from each other, resulting in the recovery of progeny seed that contain the trait genes (heterologous polynucleotides) with no morphogenic genes/herbicide genes. See, Miller et al. Transgenic Res 11(4):381-96.

The methods provided herein rely upon the use of bacteria-mediated and/or biolistic-mediated gene transfer to produce regenerable plant cells having an incorporated nucleotide sequence of interest. Bacterial strains useful in the methods of the disclosure include, but are not limited to, a disarmed Agrobacteria, an Ochrobactrum bacteria or a Rhizobiaceae bacteria. Disarmed Agrobacteria useful in the present methods include, but are not limited to, AGL-1, EHA105, GV3101, LBA4404, and LBA4404 THY-. Ochrobactrum bacterial strains useful in the present methods include, but are not limited to, those disclosed in U.S. Pat. Pub. No. US20180216123 incorporated herein by reference in its entirety. Rhizobiaceae bacterial strains useful in the present methods include, but are not limited to, those disclosed in U.S. Pat. No. 9,365,859 incorporated herein by reference in its entirety.

Also embodied is a plant with the described expression cassette stably incorporated into the genome of the plant, a seed of the plant, wherein the seed comprises the expression cassette. Further embodied is a plant wherein a gene or gene product of a heterologous polynucleotide or a polynucleotide of interest that confers a nutritional enhancement, increased yield, abiotic stress tolerance, drought tolerance, cold tolerance, herbicide tolerance, pest resistance, pathogen resistance, insect resistance, nitrogen use efficiency (NUE), disease resistance, or an ability to alter a metabolic pathway. A plant wherein expression of a heterologous polynucleotide or a polynucleotide of interest alters the phenotype of said plant is also embodied.

The disclosure encompasses isolated or substantially purified nucleic acid compositions. An “isolated” or “purified” nucleic acid molecule or biologically active portion thereof is substantially free of other cellular material or culture medium when produced by recombinant techniques or substantially free of chemical precursors or other chemicals when chemically synthesized. An “isolated” nucleic acid is substantially free of sequences (including protein encoding sequences) that naturally flank the nucleic acid (i.e., sequences located at the 5′ and 3′ ends of the nucleic acid) in the genomic DNA of the organism from which the nucleic acid is derived. For example, in various aspects, the isolated nucleic acid molecule can contain less than about 5 kb, 4 kb, 3 kb, 2 kb, 1 kb, 0.5 kb or 0.1 kb of nucleotide sequences that naturally flank the nucleic acid molecule in genomic DNA of the cell from which the nucleic acid is derived.

As used herein, the term “fragment” refers to a portion of the nucleic acid sequence. Fragments of sequences useful in the methods of the present disclosure retain the biological activity of the nucleic acid sequence. Alternatively, fragments of a nucleotide sequence that are useful as hybridization probes may not necessarily retain biological activity. Fragments of a nucleotide sequence disclosed herein may range from at least about 20, 25, 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1025, 1050, 1075, 1100, 1125, 1150, 1175, 1200, 1225, 1250, 1275, 1300, 1325, 1350, 1375, 1400, 1425, 1450, 1475, 1500, 1525, 1550, 1575, 1600, 1625, 1650, 1675, 1700, 1725, 1750, 1775, 1800, 1825, 1850, 1875, or 1900 nucleotides, and up to the full length of the subject sequence. A biologically active portion of a nucleotide sequence can be prepared by isolating a portion of the sequence and assessing the activity of the portion.

Fragments and variants of nucleotide sequences and the proteins encoded thereby useful in the methods of the present disclosure are also encompassed. As used herein, the term “fragment” refers to a portion of a nucleotide sequence and hence the protein encoded thereby or a portion of an amino acid sequence. Fragments of a nucleotide sequence may encode protein fragments that retain the biological activity of the native protein. Alternatively, fragments of a nucleotide sequence useful as hybridization probes generally do not encode fragment proteins retaining biological activity. Thus, fragments of a nucleotide sequence may range from at least about 20 nucleotides, about 50 nucleotides, about 100 nucleotides, and up to the full-length nucleotide sequence encoding the proteins useful in the methods of the present disclosure.

As used herein, the term “variants” is means sequences having substantial similarity with a promoter sequence disclosed herein. A variant comprises a deletion and/or addition of one or more nucleotides at one or more internal sites within the native polynucleotide and/or a substitution of one or more nucleotides at one or more sites in the native polynucleotide. As used herein, a “native” nucleotide sequence comprises a naturally occurring nucleotide sequence. For nucleotide sequences, naturally occurring variants can be identified with the use of well-known molecular biology techniques, such as, for example, with polymerase chain reaction (PCR) and hybridization techniques as outlined herein.

Variant nucleotide sequences also include synthetically derived nucleotide sequences, such as those generated, for example, by using site-directed mutagenesis. Generally, variants of a nucleotide sequence disclosed herein will have at least 40%, 50%, 60%, 65%, 70%, 75%, 80%, 85%, 90%, 91%, 92%, 93%, 94%, to 95%, 96%, 97%, 98%, 99% or more sequence identity to that nucleotide sequence as determined by sequence alignment programs described elsewhere herein using default parameters. Biologically active variants of a nucleotide sequence disclosed herein are also encompassed. Biological activity may be measured by using techniques such as Northern blot analysis, reporter activity measurements taken from transcriptional fusions, and the like. See, for example, Sambrook, et al., (1989) Molecular Cloning: A Laboratory Manual (2d ed., Cold Spring Harbor Laboratory Press, Cold Spring Harbor, N.Y.), hereinafter “Sambrook,” herein incorporated by reference in its entirety. Alternatively, levels of a reporter gene such as green fluorescent protein (GFP) or yellow fluorescent protein (YFP) or the like produced under the control of a promoter operably linked to a nucleotide fragment or variant can be measured. See, for example, Matz et al. (1999) Nature Biotechnology 17:969-973; U.S. Pat. No. 6,072,050, herein incorporated by reference in its entirety; Nagai, et al., (2002) Nature Biotechnology 20(1):87-90. Variant nucleotide sequences also encompass sequences derived from a mutagenic and recombinogenic procedure such as DNA shuffling. With such a procedure, one or more different nucleotide sequences can be manipulated to create a new nucleotide sequence. In this manner, libraries of recombinant polynucleotides are generated from a population of related sequence polynucleotides comprising sequence regions that have substantial sequence identity and can be homologously recombined in vitro or in vivo. Strategies for such DNA shuffling are known in the art. See, for example, Stemmer, (1994) Proc. Natl. Acad. Sci. USA 91:10747-10751; Stemmer, (1994) Nature 370:389 391; Crameri, et al., (1997) Nature Biotech. 15:436-438; Moore, et al., (1997) J. Mol. Biol. 272:336-347; Zhang, et al., (1997) Proc. Natl. Acad. Sci. USA 94:4504-4509; Crameri, et al., (1998) Nature 391:288-291 and U.S. Pat. Nos. 5,605,793 and 5,837,458, herein incorporated by reference in their entirety.

Methods for mutagenesis and nucleotide sequence alterations are well known in the art. See, for example, Kunkel, (1985) Proc. Natl. Acad. Sci. USA 82:488-492; Kunkel, et al., (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein, herein incorporated by reference in their entirety. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be optimal.

The nucleotide sequences of the present disclosure can be used to isolate corresponding sequences from other organisms, particularly other plants, more particularly other monocots or dicots. In this manner, methods such as PCR, hybridization and the like can be used to identify such sequences based on their sequence homology to the sequences set forth herein. Sequences isolated based on their sequence identity to the entire sequences set forth herein or to fragments thereof are encompassed by the present disclosure.

In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art and are disclosed in, Sambrook, supra. See also, Innis, et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York), herein incorporated by reference in their entirety. Known methods of PCR include, but are not limited to, methods using paired primers, nested primers, single specific primers, degenerate primers, gene-specific primers, vector-specific primers, partially-mismatched primers and the like. In hybridization techniques, all or part of a known nucleotide sequence is used as a probe that selectively hybridizes to other corresponding nucleotide sequences present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides and may be labeled with a detectable group such as 32P or any other detectable marker. Thus, for example, probes for hybridization can be made by labeling synthetic oligonucleotides based on the sequences of the present disclosure. Methods for preparation of probes for hybridization and for construction of genomic libraries are generally known in the art and are disclosed in Sambrook, supra.

In general, sequences that have activity and hybridize to the sequences disclosed herein will be at least 40% to 50% homologous, about 60%, 70%, 80%, 85%, 90%, 95% to 98% homologous or more with the disclosed sequences. That is, the sequence similarity of sequences may range, sharing at least about 40% to 50%, about 60% to 70%, and about 80%, 85%, 90%, 95% to 98% sequence similarity.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller, (1988) CABIOS 4:11-17; the algorithm of Smith, et al., (1981) Adv. Appl. Math. 2:482; the algorithm of Needleman and Wunsch, (1970) J. Mol. Biol. 48:443-453; the algorithm of Pearson and Lipman, (1988) Proc. Natl. Acad. Sci. 85:2444-2448; the algorithm of Karlin and Altschul, (1990) Proc. Natl. Acad. Sci. USA 872:264, modified as in Karlin and Altschul, (1993) Proc. Natl. Acad. Sci. USA 90:5873-5877, herein incorporated by reference in their entirety. Computer implementations of these mathematical algorithms are well known in the art and can be utilized for comparison of sequences to determine sequence identity.

As used herein, “sequence identity” or “identity” in the context of two nucleic acid or polypeptide sequences refers to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically, this involves scoring a conservative substitution as a partial rather than a full mismatch, thereby increasing the percentage sequence identity. Thus, for example, where an identical amino acid is given a score of one and a non-conservative substitution is given a score of zero, a conservative substitution is given a score between zero and one. The scoring of conservative substitutions is calculated, e.g., as implemented in the program PC/GENE (Intelligenetics, Mountain View, Calif.).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The term “substantial identity” of polynucleotide sequences means that a polynucleotide comprises a sequence that has at least 70% sequence identity, optimally at least 80%, more optimally at least 90% and most optimally at least 95%, compared to a reference sequence using an alignment program using standard parameters. One of skill in the art will recognize that these values can be appropriately adjusted to determine corresponding identity of proteins encoded by two nucleotide sequences by considering codon degeneracy, amino acid similarity, reading frame positioning and the like. Substantial identity of amino acid sequences for these purposes normally means sequence identity of at least 60%, 70%, 80%, 90% and at least 95%.

Another indication that nucleotide sequences are substantially identical is if two molecules hybridize to each other under stringent conditions. Generally, stringent conditions are selected to be about 5° C. lower than the Tm for the specific sequence at a defined ionic strength and pH. However, stringent conditions encompass temperatures in the range of about 1° C. to about 20° C. lower than the Tm, depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

The methods, sequences, and genes disclosed herein are useful for genetic engineering of plants, e.g. to produce a transformed or transgenic plant, to express a phenotype of interest. As used herein, the terms “transformed plant” and “transgenic plant” refer to a plant that comprises within its genome a heterologous polynucleotide. Generally, the heterologous polynucleotide is stably integrated within the genome of a transgenic or transformed plant such that the polynucleotide is passed on to successive generations. The heterologous polynucleotide may be integrated into the genome alone or as part of a recombinant DNA construct. It is to be understood that as used herein the term “transgenic” includes any cell, cell line, callus, tissue, plant part or plant the genotype of which has been altered by the presence of a heterologous nucleic acid including those transgenics initially so altered as well as those created by sexual crosses or asexual propagation from the initial transgenic.

A transgenic “event” is produced by transformation of plant cells with a heterologous DNA construct, including a nucleic acid expression cassette that comprises a gene of interest, the regeneration of a population of plants resulting from the insertion of the transferred gene into the genome of the plant and selection of a plant characterized by insertion into a particular genome location. An event is characterized phenotypically by the expression of the inserted gene. At the genetic level, an event is part of the genetic makeup of a plant. The term “event” also refers to progeny produced by a sexual cross between the transformant and another plant wherein the progeny include the heterologous DNA.

The term “plant” refers to whole plants, plant organs (e.g., leaves, stems, roots, etc.), plant tissues, plant cells, plant parts, seeds, propagules, embryos, and progeny of the same. Plant cells can be differentiated or undifferentiated (e.g. callus, undifferentiated callus, immature and mature embryos, immature zygotic embryo, immature cotyledon, embryonic axis, suspension culture cells, protoplasts, leaf, leaf cells, root cells, phloem cells and pollen). Plant cells include, without limitation, cells from seeds, suspension cultures, explants, immature embryos, embryos, zygotic embryos, somatic embryos, embryogenic callus, meristem, somatic meristems, meristematic regions, organogenic callus, callus tissue, protoplasts, embryos derived from mature ear-derived seed, leaves, leaf bases, leaves from mature plants, leaf tips, immature inflorescences, tassel, immature ear, silks, cotyledons, immature cotyledons, embryonic axes, cells from leaves, cells from stems, cells from roots, cells from shoots, roots, shoots, gametophytes, sporophytes, pollen, microspores, multicellular structures (MCS), regenerable plant structures (RPS), and embryo-like structures.

Plant parts include differentiated and undifferentiated tissues including, but not limited to the following: roots, stems, shoots, leaves, pollen, seeds, tumor tissue and various forms of cells and culture (e.g., single cells, protoplasts, embryos and callus tissue). The plant tissue may be in a plant or in a plant organ, tissue or cell culture.

Grain is intended to mean the mature seed produced by commercial growers for purposes other than growing or reproducing the species. Progeny, variants and mutants of the regenerated plants are also included within the scope of the disclosure, provided these progeny, variants and mutants comprise the introduced polynucleotides.

The present disclosure also includes plants obtained by any of the methods disclosed herein. The present disclosure also includes seeds from a plant obtained by any of the methods disclosed herein.

The methods of the present disclosure may be used for transformation of plant species, including, but not limited to, alfalfa, soybean, cotton, sunflower, cassava, common bean, cowpea, tomato, potato, beet, grape, Eucalyptus, poplar, pine, douglas fir, citrus, papaya, cacao, cucumber, apple, Capsicum, melon, and Brassica. In an aspect, dicot plants used in the methods of the present disclosure, include, but are not limited to, kale, cauliflower, broccoli, mustard plant, cabbage, pea, clover, alfalfa, broad bean, tomato, peanut, cassava, soybean, canola, sunflower, safflower, tobacco, Arabidopsis, or cotton.

Higher plants, e.g., classes of Angiospermae and Gymnospermae may be used the methods of the present disclosure. Plants of suitable species useful in the methods of the present disclosure may come from the families Acanthaceae, Alliaceae, Alstroemeriaceae, Amaryllidaceae, Apocynaceae, Arecaceae, Asteraceae, Berberidaceae, Bixaceae, Brassicaceae, Bromeliaceae, Cannabaceae, Caryophyllaceae, Cephalotaxaceae, Chenopodiaceae, Colchicaceae, Cucurbitaceae, Dioscoreaceae, Ephedraceae, Erythroxylaceae, Euphorbiaceae, Fabaceae, Lamiaceae, Linaceae, Lycopodiaceae, Malvaceae, Melanthiaceae, Musaceae, Myrtaceae, Nyssaceae, Papaveraceae, Pinaceae, Plantaginaceae, Rosaceae, Rubiaceae, Salicaceae, Sapindaceae, Solanaceae, Taxaceae, Theaceae, and Vitaceae. Plants from members of the genus Abelmoschus, Abies, Acer, Allium, Alstroemeria, Ananas, Andrographis, Andropogon, Artemisia, Atropa, Berberis, Beta, Bixa, Brassica, Calendula, Camellia, Camptotheca, Cannabis, Capsicum, Carthamus, Catharanthus, Cephalotaxus, Chrysanthemum, Cinchona, Citrullus, Coffea, Colchicum, Coleus, Cucumis, Cucurbita, Cynodon, Datura, Dianthus, Digitalis, Dioscorea, Elaeis, Ephedra, Erianthus, Erythroxylum, Eucalyptus, Festuca, Fragaria, Galanthus, Glycine, Gossypium, Helianthus, Hevea, Hyoscyamus, Jatropha, Lactuca, Linum, Lupinus, Lycopersicon, Lycopodium, Manihot, Medicago, Mentha, Musa, Nicotiana, Papaver, Parthenium, Pennisetum, Petunia, Phalaris, Phleum, Pinus, Poa, Poinsettia, Populus, Rauwolfia, Ricinus, Rosa, Saccharum, Salix, Sanguinaria, Scopolia, Solanum, Spinacea, Tanacetum, Taxus, Theobroma, Uniola, Veratrum, Vinca, and Vitis.

Plants important or interesting for agriculture, horticulture, biomass production (for production of liquid fuel molecules and other chemicals), and/or forestry may be used in the methods of the disclosure. Non-limiting examples include, for instance, Populus balsamifera (poplar), cotton (Gossypium barbadense, Gossypium hirsutum), Helianthus annuus (sunflower), Medicago sativa (alfalfa), Beta vulgaris (sugarbeet), Erianthus spp., Salix spp. (willow), Eucalyptus spp. (eucalyptus, including E. grandis (and its hybrids, known as “urograndis”), E. globulus, E. camaldulensis, E. tereticornis, E. viminalis, E. nitens, E. saligna and E. urophylla), Carthamus tinctorius (safflower), Jatropha curcas (jatropha), Ricinus communis (castor), Manihot esculenta (cassava), Solanum lycopersicum (tomato), Lactuca sativa (lettuce), Phaseolus vulgaris (green beans), Phaseolus limensis (lima beans), Lathyrus spp. (peas), Solanum tuberosum (potato), Brassica spp. (B. napus (canola), B. rapa, B. juncea), Brassica oleracea (broccoli, cauliflower, brussel sprouts), Camellia sinensis (tea), Fragaria ananassa (strawberry), Theobroma cacao (cocoa), Coffea arabica (coffee), Vitis vinifera (grape), Ananas comosus (pineapple), Capsicum annum (hot & sweet pepper), Arachis hypogaea (peanuts), Ipomoea batatus (sweet potato), Cocos nucifera (coconut), Citrus spp. (citrus trees), Persea americana (avocado), fig (Ficus casica), guava (Psidium guajava), mango (Mangifera indica), olive (Olea europaea), Carica papaya (papaya), Anacardium occidentale (cashew), Macadamia integrifolia (macadamia tree), Prunus amygdalus (almond), Allium cepa (onion), Cucumis melo (musk melon), Cucumis sativus (cucumber), Cucumis cantalupensis (cantaloupe), Cucurbita maxima (squash), Cucurbita moschata (squash), Spinacea oleracea (spinach), Citrullus lanatus (watermelon), Abelmoschus esculentus (okra), Solanum melongena (eggplant), Cyamopsis tetragonoloba (guar bean), Ceratonia siliqua (locust bean), Trigonella foenum-graecum (fenugreek), Vigna radiata (mung bean), Vigna unguiculata (cowpea), Vicia faba (fava bean), Cicer arietinum (chickpea), Lens culinaris (lentil), Papaver somniferum (opium poppy), Papaver orientale, Taxus baccata, Taxus brevifolia, Artemisia annua, Cannabis sativa, Camptotheca acuminate, Catharanthus roseus, Vinca rosea, Cinchona officinalis, Colchicum autumnale, Veratrum californica, Digitalis lanata, Digitalis purpurea, Dioscorea spp., Andrographis paniculata, Atropa belladonna, Datura stomonium, Berberis spp., Cephalotaxus spp., Ephedra sinica, Ephedra spp., Erythroxylum coca, Galanthus wornorii, Scopolia spp., Lycopodium serratum (Huperzia serrata), Lycopodium spp., Rauwolfia serpentina, Rauwolfia spp., Sanguinaria canadensis, Hyoscyamus spp., Calendula officinalis, Chrysanthemum parthenium, Coleus forskohlii, Tanacetum parthenium, Parthenium argentatum (guayule), Hevea spp. (rubber), Mentha spicata (mint), Mentha piperita (mint), Bixa orellana (achiote), Alstroemeria spp., Rosa spp. (rose), Rhododendron spp. (azalea), Macrophylla hydrangea (hydrangea), Hibiscus rosasanensis (hibiscus), Tulipa spp. (tulips), Narcissus spp. (daffodils), Petunia hybrida (petunias), Dianthus caryophyllus (carnation), Euphorbia pulcherrima (poinsettia), chrysanthemum, Nicotiana tabacum (tobacco), Lupinus albus (lupin), Populus tremuloides (aspen), Pinus spp. (pine), Abies spp. (fir), Acer spp. (maple), and conifers.

Conifers may be used in the methods of the present disclosure and include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Eastern or Canadian hemlock (Tsuga canadensis); Western hemlock (Tsuga heterophylla); Mountain hemlock (Tsuga mertensiana); Tamarack or Larch (Larix occidentalis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis).

In specific aspects, plants useful in the methods of the present disclosure are crop plants (for example, alfalfa, sunflower, Brassica, soybean, cotton, safflower, peanut, tobacco, etc.). Other plants useful in the methods of the present disclosure include cassava, common bean, cowpea, tomato, potato, beet, grape, Eucalyptus, poplar, pine, douglas fir, citrus, papaya, cacao, cucumber, apple, Capsicum, and melon.

Additional heterologous coding sequences, heterologous polynucleotides, and polynucleotides of interest may be used in the methods of the disclosure for varying the phenotype of a plant. Various changes in phenotype are of interest including modifying expression of a gene in a plant, altering a plant's pathogen or insect defense mechanism, increasing a plant's tolerance to herbicides, altering plant development to respond to environmental stress, modulating the plant's response to salt, temperature (hot and cold), drought and the like. These results can be achieved by the expression of a heterologous nucleotide sequence of interest comprising an appropriate gene product. In specific aspects, the heterologous nucleotide sequence of interest is an endogenous plant sequence whose expression level is increased in the plant or plant part. Results can be achieved by providing for altered expression of one or more endogenous gene products, particularly hormones, receptors, signaling molecules, enzymes, transporters or cofactors or by affecting nutrient uptake in the plant. These changes result in a change in phenotype of the transformed plant.

General categories of heterologous polynucleotides or nucleotide sequences of interest for use in the methods of the present disclosure include, for example, those genes involved in information, such as zinc fingers, those involved in communication, such as kinases and those involved in housekeeping, such as heat shock proteins. More specific categories of transgenes (heterologous polynucleotides or nucleotide sequences of interest), for example, include genes encoding important traits for agronomics, insect resistance, disease resistance, herbicide resistance, environmental stress resistance (altered tolerance to cold, salt, drought, etc.) and grain characteristics. Still other categories of transgenes include genes for inducing expression of exogenous products such as enzymes, cofactors, and hormones from plants and other eukaryotes as well as prokaryotic organisms. It is recognized that any gene or polynucleotide of interest can be operably linked to a promoter and expressed in a plant using the methods disclosed herein.

Many agronomic traits can affect “yield”, including without limitation, plant height, pod number, pod position on the plant, number of internodes, incidence of pod shatter, grain size, efficiency of nodulation and nitrogen fixation, efficiency of nutrient assimilation, resistance to biotic and abiotic stress, carbon assimilation, plant architecture, resistance to lodging, percent seed germination, seedling vigor, and juvenile traits. Other traits that can affect yield include, efficiency of germination (including germination in stressed conditions), growth rate (including growth rate in stressed conditions), ear number, seed number per ear, seed size, composition of seed (starch, oil, protein) and characteristics of seed fill. Also of interest is the generation of transgenic plants that demonstrate desirable phenotypic properties that may or may not confer an increase in overall plant yield. Such properties include enhanced plant morphology, plant physiology or improved components of the mature seed harvested from the transgenic plant.

“Increased yield” of a transgenic plant of the present disclosure may be evidenced and measured in a number of ways, including test weight, seed number per plant, seed weight, seed number per unit area (i.e. seeds, or weight of seeds, per acre), bushels per acre, tons per acre, kilo per hectare. For example, maize yield may be measured as production of shelled corn kernels per unit of production area, e.g. in bushels per acre or metric tons per hectare, often reported on a moisture adjusted basis, e.g., at 15.5% moisture. Increased yield may result from improved utilization of key biochemical compounds, such as nitrogen, phosphorous and carbohydrate, or from improved tolerance to environmental stresses, such as cold, heat, drought, salt, and attack by pests or pathogens. Trait-enhancing recombinant DNA may also be used to provide transgenic plants having improved growth and development, and ultimately increased yield, as the result of modified expression of plant growth regulators or modification of cell cycle or photosynthesis pathways.

An “enhanced trait” as used herein describing the aspects of the present disclosure includes improved or enhanced water use efficiency or drought tolerance, osmotic stress tolerance, high salinity stress tolerance, heat stress tolerance, enhanced cold tolerance, including cold germination tolerance, increased yield, improved seed quality, enhanced nitrogen use efficiency, early plant growth and development, late plant growth and development, enhanced seed protein, and enhanced seed oil production.

Multiple genes of interest (heterologous polynucleotides or nucleotide sequences of interest) can be used in the methods of the disclosure and expressed in a plant, for example insect resistance traits herbicide resistance, fungal resistance, virus resistance, stress tolerance, disease resistance, male sterility, stalk strength, and the like) or output traits (e.g., increased yield, modified starches, improved oil profile, balanced amino acids, high lysine or methionine, increased digestibility, improved fiber quality, drought resistance, nutritional enhancement, and the like).

In an aspect, the methods of the disclosure can be used to transform vegetative plant organs and their composite tissues including but are not limited to leaf explants, leaf primordia, stipule, cotyledons, cotyledonary nodes, mesocotyl, stem explants, primary roots, lateral secondary roots, root segments, buds, and meristems, including but not limited to apical meristems, root meristems, secondary meristems, axillary meristems, and floral meristems with insect resistance genes (heterologous polynucleotides or nucleotide sequences of interest) that encode resistance to pests that have great yield drag such as rootworm, cutworm, European corn borer and the like. Such genes (heterologous polynucleotides or nucleotide sequences of interest) include, for example, Bacillus thuringiensis toxic protein genes, U.S. Pat. Nos. 5,366,892; 5,747,450; 5,736,514; 5,723,756; 5,593,881 and Geiser, et al., (1986) Gene 48:109, the disclosures of which are herein incorporated by reference in their entirety. Genes (heterologous polynucleotides or nucleotide sequences of interest) encoding disease resistance traits can also be used in the methods of the disclosure including, for example, detoxification genes, such as those which detoxify fumonisin (U.S. Pat. No. 5,792,931); avirulence (avr) and disease resistance (R) genes (Jones, et al., (1994) Science 266:789; Martin, et al., (1993) Science 262:1432; and Mindrinos, et al., (1994) Cell 78:1089), herein incorporated by reference in their entirety.

Herbicide resistance traits (heterologous polynucleotides or nucleotide sequences of interest) can be used in the methods of the disclosure including genes coding for resistance to herbicides that act to inhibit the action of acetolactate synthase (ALS), in particular the sulfonylurea-type herbicides (e.g., the acetolactate synthase (ALS) gene containing mutations leading to such resistance, in particular the S4 and/or Hra mutations), genes coding for resistance to herbicides that act to inhibit action of glutamine synthase, such as phosphinothricin or basta (e.g., the bar gene), genes coding for resistance to glyphosate (e.g., the EPSPS gene and the GAT gene; see, for example, US Patent Application Publication Number 2004/0082770 and WO 03/092360, herein incorporated by reference in their entirety) or other such genes known in the art. The bar gene encodes resistance to the herbicide basta, the nptll gene encodes resistance to the antibiotics kanamycin and geneticin and the ALS-gene mutants encode resistance to the herbicide chlorsulfuron any and all of which can be operably linked to a promoter and used in the methods of the disclosure.

Glyphosate resistance is imparted by mutant 5-enolpyruvl-3-phosphikimate synthase (EPSPS) and aroA genes which can be operably linked to a promoter and used in the methods of the disclosure. See, for example, U.S. Pat. No. 4,940,835 to Shah, et al., which discloses the nucleotide sequence of a form of EPSPS which can confer glyphosate resistance. U.S. Pat. No. 5,627,061 to Barry, et al., also describes genes encoding EPSPS enzymes which can be operably linked to a promoter and used in the methods of the disclosure. See also, U.S. Pat. Nos. 6,248,876 B1; 6,040,497; 5,804,425; 5,633,435; 5,145,783; 4,971,908; 5,312,910; 5,188,642; 4,940,835; 5,866,775; 6,225,114 B 1; 6,130,366; 5,310,667; 4,535,060; 4,769,061; 5,633,448; 5,510,471; Re. 36,449; RE 37,287 E and 5,491,288 and international publications WO 97/04103; WO 97/04114; WO 00/66746; WO 01/66704; WO 00/66747 and WO 00/66748, which are incorporated herein by reference in their entirety. Glyphosate resistance is also imparted to plants that express a gene that encodes a glyphosate oxido-reductase enzyme as described more fully in U.S. Pat. Nos. 5,776,760 and 5,463,175, which are incorporated herein by reference in their entirety. Glyphosate resistance can also be imparted to plants by the over expression of genes encoding glyphosate N-acetyltransferase. See, for example, U.S. patent application Ser. Nos. 11/405,845 and 10/427,692, herein incorporated by reference in their entirety. Sterility genes (heterologous polynucleotides or nucleotide sequences of interest) can be used in the methods of the disclosure to provide an alternative to physical detasseling. Examples of genes used in such ways include male tissue-preferred genes and genes with male sterility phenotypes such as QM, described in U.S. Pat. No. 5,583,210, herein incorporated by reference in its entirety. Other genes which can be operably linked to a promoter and used in the methods of the disclosure include kinases and those encoding compounds toxic to either male or female gametophytic development.

Commercial traits can also be produced using the methods of the disclosure that could increase for example, starch for ethanol production, or provide expression of proteins. Another important commercial use of transformed plants is the production of polymers and bioplastics such as described in U.S. Pat. No. 5,602,321, herein incorporated by reference in its entirety. Genes such as β-Ketothiolase, PHBase (polyhydroxybutyrate synthase), and acetoacetyl-CoA reductase which can be operably linked to a promoter and used in the methods of the disclosure (see, Schubert, et al., (1988) J. Bacterial. 170:5837-5847, herein incorporated by reference in its entirety) facilitate expression of polyhydroxyalkanoates (PHAs).

Numerous trait genes (heterologous polynucleotides or nucleotide sequences of interest) are known in the art and can be used in the methods disclosed herein. By way of illustration, without intending to be limiting, trait genes (heterologous polynucleotides) that confer resistance to insects or diseases, trait genes (heterologous polynucleotides) that confer resistance to a herbicide, trait genes (heterologous polynucleotides) that confer or contribute to an altered grain characteristic, such as altered fatty acids, altered phosphorus content, altered carbohydrates or carbohydrate composition, altered antioxidant content or composition, or altered essential seed amino acids content or composition are examples of the types of trait genes (heterologous polynucleotides) which can be operably linked to a promoter for expression in plants transformed by the methods disclosed herein. Additional genes known in the art may be included in the expression cassettes useful in the methods disclosed herein. Non-limiting examples include genes that create a site for site specific DNA integration, genes that affect abiotic stress resistance (including but not limited to flowering, ear and seed development, enhancement of nitrogen utilization efficiency, altered nitrogen responsiveness, drought resistance or tolerance, cold resistance or tolerance, and salt resistance or tolerance) and increased yield under stress, or other genes and transcription factors that affect plant growth and agronomic traits such as yield, flowering, plant growth and/or plant structure.

The methods of the disclosure can be used to transform a plant with a heterologous nucleotide sequence that is an antisense sequence for a targeted gene. As used herein, “antisense orientation” includes reference to a polynucleotide sequence that is operably linked to a promoter in an orientation where the antisense strand is transcribed. The antisense strand is sufficiently complementary to an endogenous transcription product such that translation of the endogenous transcription product is often inhibited. “Operably linked” refers to the association of two or more nucleic acid fragments on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The terminology “antisense DNA nucleotide sequence” is intended to mean a sequence that is in inverse orientation to the 5′-to-3′ normal orientation of that nucleotide sequence. When delivered into a plant cell, expression of the antisense DNA sequence prevents normal expression of the DNA nucleotide sequence for the targeted gene. The antisense nucleotide sequence encodes an RNA transcript that is complementary to and capable of hybridizing to the endogenous messenger RNA (mRNA) produced by transcription of the DNA nucleotide sequence for the targeted gene. In this case, production of the native protein encoded by the targeted gene is inhibited to achieve a desired phenotypic response. Modifications of the antisense sequences may be made as long as the sequences hybridize to and interfere with expression of the corresponding mRNA. In this manner, antisense constructions having 70%, 80%, 85% sequence identity to the corresponding antisense sequences may be used. Furthermore, portions of the antisense nucleotides may be used to disrupt the expression of the target gene. Generally, sequences of at least 50 nucleotides, 100 nucleotides, 200 nucleotides or greater may be used. Thus, the promoter sequences disclosed herein may be operably linked to antisense DNA sequences to reduce or inhibit expression of a native protein in the plant.

“RNAi” refers to a series of related techniques to reduce the expression of genes (see, for example, U.S. Pat. No. 6,506,559, herein incorporated by reference in its entirety). Older techniques referred to by other names are now thought to rely on the same mechanism but are given different names in the literature. These include “antisense inhibition,” the production of antisense RNA transcripts capable of suppressing the expression of the target protein and “co-suppression” or “sense-suppression,” which refer to the production of sense RNA transcripts capable of suppressing the expression of identical or substantially similar foreign or endogenous genes (U.S. Pat. No. 5,231,020, incorporated herein by reference in its entirety). Such techniques rely on the use of constructs resulting in the accumulation of double stranded RNA with one strand complementary to the target gene to be silenced. The methods of the disclosure may be used to express constructs that will result in RNA interference including microRNAs and siRNAs.

As used herein, the terms “promoter” or “transcriptional initiation region” mean a regulatory region of DNA usually comprising a TATA box or a DNA sequence capable of directing RNA polymerase II to initiate RNA synthesis at the appropriate transcription initiation site for a particular coding sequence. A promoter may additionally comprise other recognition sequences generally positioned upstream or 5′ to the TATA box or the DNA sequence capable of directing RNA polymerase II to initiate RNA synthesis, referred to as upstream promoter elements, which influence the transcription initiation rate. It is recognized that having identified the nucleotide sequences for the promoter regions disclosed herein, it is within the state of the art to isolate and identify further promoters in the 5′ untranslated region upstream from the particular promoter regions identified herein. Additionally, chimeric promoters may be provided. Such chimeras include portions of the promoter sequence fused to fragments and/or variants of heterologous transcriptional regulatory regions. Thus, the promoter regions disclosed herein can comprise upstream promoters such as, those responsible for tissue and temporal expression of the coding sequence, enhancers and the like.

As used herein, the term “regulatory element” also refers to a sequence of DNA, usually, but not always, upstream (5′) to the coding sequence of a structural gene, which includes sequences which control the expression of the coding region by providing the recognition for RNA polymerase and/or other factors required for transcription to start at a particular site. An example of a regulatory element that provides for the recognition for RNA polymerase or other transcriptional factors to ensure initiation at a particular site is a promoter element. A promoter element comprises a core promoter element, responsible for the initiation of transcription, as well as other regulatory elements that modify gene expression. It is to be understood that nucleotide sequences, located within introns or 3′ of the coding region sequence may also contribute to the regulation of expression of a coding region of interest. Examples of suitable introns include, but are not limited to, the maize IVS6 intron, or the maize actin intron. A regulatory element may also include those elements located downstream (3′) to the site of transcription initiation, or within transcribed regions, or both. In the context of the present disclosure a post-transcriptional regulatory element may include elements that are active following transcription initiation, for example translational and transcriptional enhancers, translational and transcriptional repressors and mRNA stability determinants.

A “heterologous nucleotide sequence”, “heterologous polynucleotide of interest”, or “heterologous polynucleotide” as used throughout the disclosure, is a sequence that is not naturally occurring with or operably linked to a promoter sequence. While this nucleotide sequence is heterologous to the promoter sequence, it may be homologous or native or heterologous or foreign to the plant host. Likewise, the promoter sequence may be homologous or native or heterologous or foreign to the plant host and/or the polynucleotide of interest.

It is recognized that to increase transcription levels, enhancers may be. Enhancers are nucleotide sequences that act to increase the expression of a promoter region. Enhancers are known in the art and include the SV40 enhancer region, the 35S enhancer element and the like. Some enhancers are also known to alter normal promoter expression patterns, for example, by causing a promoter to be expressed constitutively when without the enhancer, the same promoter is expressed only in one specific tissue or a few specific tissues.

Modifications of promoter sequences can provide for a range of expression of a heterologous nucleotide sequence. Thus, they may be modified to be weak promoters or strong promoters. Generally, a “weak promoter” means a promoter that drives expression of a coding sequence at a low level. A “low level” of expression is intended to mean expression at levels of about 1/10,000 transcripts to about 1/100,000 transcripts to about 1/500,000 transcripts. Conversely, a strong promoter drives expression of a coding sequence at a high level, or at about 1/10 transcripts to about 1/100 transcripts to about 1/1,000 transcripts.

The transformation methods disclosed herein are useful in the genetic manipulation of any plant, thereby resulting in a change in phenotype of the transformed plant.

The term “operably linked” means that the transcription or translation of a heterologous nucleotide sequence is under the influence of a promoter sequence. In this manner, the nucleotide sequences for the promoters may be provided in expression cassettes along with heterologous nucleotide sequences of interest for expression in the plant of interest, more particularly for expression in the reproductive tissue of the plant.

In one aspect of the disclosure, expression cassettes comprise a transcriptional initiation region comprising a promoter nucleotide sequence or variants or fragments thereof, operably linked to a morphogenic gene and/or a heterologous nucleotide sequence. Such an expression cassette can be provided with a plurality of restriction sites for insertion of the nucleotide sequence to be under the transcriptional regulation of the regulatory regions. The expression cassette may additionally contain selectable marker genes as well as 3′ termination regions.

The expression cassette can include, in the 5′-3′ direction of transcription, a transcriptional initiation region (i.e., a promoter, or variant or fragment thereof), a translational initiation region, a heterologous nucleotide sequence of interest, a translational termination region and optionally, a transcriptional termination region functional in the host organism. The regulatory regions (i.e., promoters, transcriptional regulatory regions, and translational termination regions) and/or the polynucleotide of the aspects may be native/analogous to the host cell or to each other. Alternatively, the regulatory regions and/or the polynucleotide of the aspects may be heterologous to the host cell or to each other. As used herein, “heterologous” in reference to a sequence is a sequence that originates from a foreign species or, if from the same species, is substantially modified from its native form in composition and/or genomic locus by deliberate human intervention. For example, a promoter operably linked to a heterologous polynucleotide is from a species different from the species from which the polynucleotide was derived or, if from the same/analogous species, one or both are substantially modified from their original form and/or genomic locus or the promoter is not the native promoter for the operably linked polynucleotide. The termination region may be native with the transcriptional initiation region, may be native with the operably linked DNA sequence of interest, may be native with the plant host, or may be derived from another source (i.e., foreign or heterologous to the promoter, the DNA sequence being expressed, the plant host, or any combination thereof). Convenient termination regions are available from the Ti-plasmid of A. tumefaciens, such as the octopine synthase and nopaline synthase termination regions. See also, Guerineau, et al., (1991) Mol. Gen. Genet. 262:141-144; Proudfoot, (1991) Cell 64:671-674; Sanfacon, et al., (1991) Genes Dev. 5:141-149; Mogen, et al., (1990) Plant Cell 2:1261-1272; Munroe, et al., (1990) Gene 91:151-158; Ballas, et al., (1989) Nucleic Acids Res. 17:7891-7903; and Joshi, et al., (1987) Nucleic Acid Res. 15:9627-9639, herein incorporated by reference in their entirety.

The expression cassette useful in the methods of the disclosure may also contain at least one additional nucleotide sequence for a gene, heterologous nucleotide sequence, heterologous polynucleotide of interest, or heterologous polynucleotide to be co-transformed into the organism. Alternatively, the additional nucleotide sequence(s) can be provided on another expression cassette.

Where appropriate, the nucleotide sequences may be optimized for increased expression in the transformed plant. That is, these nucleotide sequences can be synthesized using plant preferred codons for improved expression. See, for example, Campbell and Gowri, (1990) Plant Physiol. 92:1-11, herein incorporated by reference in its entirety, for a discussion of host-preferred codon usage. Methods are available in the art for synthesizing plant-preferred genes. See, for example, U.S. Pat. Nos. 5,380,831, 5,436,391 and Murray, et al., (1989) Nucleic Acids Res. 17:477-498, herein incorporated by reference in their entirety.

Additional sequence modifications are known to enhance gene expression in a cellular host. These include elimination of sequences encoding spurious polyadenylation signals, exon-intron splice site signals, transposon-like repeats and other such well-characterized sequences that may be deleterious to gene expression. The G-C content of the heterologous nucleotide sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences. Such leader sequences can act to enhance translation. Translation leaders are known in the art and include, without limitation: picornavirus leaders, for example, EMCV leader (Encephalomyocarditis 5′ noncoding region) (Elroy-Stein, et al., (1989) Proc. Nat. Acad. Sci. USA 86:6126-6130); potyvirus leaders, for example, TEV leader (Tobacco Etch Virus) (Allison, et al., (1986) Virology 154:9-20); MDMV leader (Maize Dwarf Mosaic Virus); human immunoglobulin heavy-chain binding protein (BiP) (Macejak, et al., (1991) Nature 353:90-94); untranslated leader from the coat protein mRNA of alfalfa mosaic virus (AMV RNA 4) (Jobling, et al., (1987) Nature 325:622-625); tobacco mosaic virus leader (TMV) (Gallie, et al., (1989) Molecular Biology of RNA, pages 237-256) and maize chlorotic mottle virus leader (MCMV) (Lommel, et al., (1991) Virology 81:382-385), herein incorporated by reference in their entirety. See, also, Della-Cioppa, et al., (1987) Plant Physiology 84:965-968, herein incorporated by reference in its entirety. Methods known to enhance mRNA stability can also be utilized, for example, introns, such as the maize Ubiquitin intron (Christensen and Quail, (1996) Transgenic Res. 5:213-218; Christensen, et al., (1992) Plant Molecular Biology 18:675-689) or the maize Adhl intron (Kyozuka, et al., (1991) Mol. Gen. Genet. 228:40-48; Kyozuka, et al., (1990)Maydica 35:353-357) and the like, herein incorporated by reference in their entirety.

The DNA expression cassettes or constructs useful in the methods of the disclosure can also include further enhancers, either translation or transcription enhancers, as may be required. These enhancer regions are well known to persons skilled in the art and can include the ATG initiation codon and adjacent sequences. The initiation codon must be in phase with the reading frame of the coding sequence to ensure translation of the entire sequence. The translation control signals and initiation codons can be from a variety of origins, both natural and synthetic. Translational initiation regions may be provided from the source of the transcriptional initiation region, or from the structural gene. The sequence can also be derived from the regulatory element selected to express the gene and can be specifically modified to increase translation of the mRNA. It is recognized that to increase transcription levels enhancers may be utilized in combination with the promoter regions of the aspects. Enhancers are known in the art and include the SV40 enhancer region, the 35S enhancer element, and the like.

In preparing the expression cassette, the various DNA fragments may be manipulated, to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites or the like. For this purpose, in vitro mutagenesis, primer repair, restriction, annealing, resubstitutions, for example, transitions and transversions, may be involved.

Reporter genes or selectable marker genes may also be included in the expression cassettes useful in the methods of the present disclosure. Examples of suitable reporter genes known in the art can be found in, for example, Jefferson, et al., (1991) in Plant Molecular Biology Manual, ed. Gelvin, et al., (Kluwer Academic Publishers), pp. 1-33; DeWet, et al., (1987) Mol. Cell. Biol. 7:725-737; Goff, et al., (1990) EMBO J. 9:2517-2522; Kain, et al., (1995) Bio Techniques 19:650-655 and Chiu, et al., (1996) Current Biology 6:325-330, herein incorporated by reference in their entirety.

Selectable marker genes for selection of transformed cells or tissues can include genes that confer antibiotic resistance or resistance to herbicides. Examples of suitable selectable marker genes include, but are not limited to, genes encoding resistance to chloramphenicol (Herrera Estrella, et al., (1983) EMBO 1 2:987-992); methotrexate (Herrera Estrella, et al., (1983) Nature 303:209-213; Meijer, et al., (1991) Plant Mol. Biol. 16:807-820); hygromycin (Waldron, et al., (1985) Plant Mol. Biol. 5:103-108 and Zhijian, et al., (1995) Plant Science 108:219-227); streptomycin (Jones, et al., (1987) Mol. Gen. Genet. 210:86-91); spectinomycin (Bretagne-Sagnard, et al., (1996) Transgenic Res. 5:131-137); bleomycin (Hille, et al., (1990) Plant Mol. Biol. 7:171-176); sulfonamide (Guerineau, et al., (1990) Plant Mol. Biol. 15:127-36); bromoxynil (Stalker, et al., (1988) Science 242:419-423); glyphosate (Shaw, et al., (1986) Science 233:478-481 and U.S. patent application Ser. Nos. 10/004,357 and 10/427,692); phosphinothricin (DeBlock, et al., (1987) EMBO J. 6:2513-2518), herein incorporated by reference in their entirety.

Other genes that could serve utility in the recovery of transgenic events would include, but are not limited to, examples such as GUS (beta-glucuronidase; Jefferson, (1987) Plant Mol. Biol. Rep. 5:387), GFP (green fluorescence protein; Chalfie, et al., (1994) Science 263:802), luciferase (Riggs, et al., (1987) Nucleic Acids Res. 15(19):8115 and Luehrsen, et al., (1992) Methods Enzymol. 216:397-414) and the maize genes encoding for anthocyanin production (Ludwig, et al., (1990) Science 247:449), herein incorporated by reference in their entirety.

As used herein, “vector” refers to a DNA molecule such as a plasmid, cosmid or bacterial phage for introducing a nucleotide construct, for example, an expression cassette or construct, into a host cell. Cloning vectors typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide tetracycline resistance, hygromycin resistance or ampicillin resistance.

The methods of the disclosure involve introducing a polypeptide or polynucleotide into a plant. As used herein, “introducing” means presenting to the plant the polynucleotide or polypeptide in such a manner that the sequence gains access to the interior of a cell of the plant. The methods of the disclosure do not depend on a particular method for introducing a sequence into a plant, only that the polynucleotide or polypeptides gains access to the interior of at least one cell of the plant. Methods for introducing polynucleotide or polypeptides into plants are known in the art including, but not limited to, stable transformation methods, transient transformation methods and virus-mediated methods.

A “stable transformation” is a transformation in which the nucleotide construct introduced into a plant integrates into the genome of the plant and is capable of being inherited by the progeny thereof. “Transient transformation” means that a polynucleotide is introduced into the plant and does not integrate into the genome of the plant or a polypeptide is introduced into a plant.

Transformation protocols as well as protocols for introducing nucleotide sequences into plants may vary depending on the type of plant or plant cell, i.e., monocot or dicot, targeted for transformation. Suitable methods of introducing nucleotide sequences into plant cells and subsequent insertion into the plant genome include microinjection (Crossway, et al., (1986) Biotechniques 4:320-334), electroporation (Riggs, et al., (1986) Proc. Natl. Acad. Sci. USA 83:5602-5606), Agrobacterium-mediated transformation (Townsend, et al., U.S. Pat. No. 5,563,055 and Zhao, et al., U.S. Pat. No. 5,981,840), direct gene transfer (Paszkowski, et al., (1984) EMBO 1 3:2717-2722) and ballistic particle acceleration (see, for example, U.S. Pat. Nos. 4,945,050; 5,879,918; 5,886,244; 5,932,782; Tomes, et al., (1995) in Plant Cell, Tissue, and Organ Culture: Fundamental Methods, ed. Gamborg and Phillips (Springer-Verlag, Berlin); McCabe, et al., (1988) Biotechnology 6:923-926) and Lecl transformation (WO 00/28058). Also see, Weissinger, et al., (1988) Ann. Rev. Genet. 22:421-477; Sanford, et al., (1987) Particulate Science and Technology 5:27-37 (onion); Christou, et al., (1988) Plant Physiol. 87:671-674 (soybean); McCabe, et al., (1988) Bio/Technology 6:923-926 (soybean); Finer and McMullen, (1991) In Vitro Cell Dev. Biol. 27P:175-182 (soybean); Singh, et al., (1998) Theor. Appl. Genet. 96:319-324 (soybean); Datta, et al., (1990) Biotechnology 8:736-740 (rice); Klein, et al., (1988) Proc. Natl. Acad. Sci. USA 85:4305-4309 (maize); Klein, et al., (1988) Biotechnology 6:559-563 (maize); U.S. Pat. Nos. 5,240,855; 5,322,783 and 5,324,646; Klein, et al., (1988) Plant Physiol. 91:440-444 (maize); Fromm, et al., (1990) Biotechnology 8:833-839 (maize); Hooykaas-Van Slogteren, et al., (1984) Nature (London) 311:763-764; U.S. Pat. No. 5,736,369 (cereals); Bytebier, et al., (1987) Proc. Natl. Acad. Sci. USA 84:5345-5349 (Liliaceae); De Wet, et al., (1985) in The Experimental Manipulation of Ovule Tissues, ed. Chapman, et al., (Longman, New York), pp. 197-209 (pollen); Kaeppler, et al., (1990) Plant Cell Reports 9:415-418 and Kaeppler, et al., (1992) Theor. Appl. Genet. 84:560-566 (whisker-mediated transformation); D'Halluin, et al., (1992) Plant Cell 4:1495-1505 (electroporation); Li, et al., (1993) Plant Cell Reports 12:250-255 and Christou and Ford, (1995) Annals of Botany 75:407-413 (rice); Osjoda, et al., (1996) Nature Biotechnology 14:745-750 (maize via Agrobacterium tumefaciens), all of which are herein incorporated by reference in their entirety. Methods and compositions for rapid plant transformation are also found in U.S. 2017/0121722, herein incorporated in its entirety by reference. Vectors useful in plant transformation are found in U.S. patent application Ser. No. 15/765,521, herein incorporated by reference in its entirety.

In specific aspects, the DNA expression cassettes or constructs can be provided to a plant using a variety of transient transformation methods. Such transient transformation methods include, but are not limited to, viral vector systems and the precipitation of the polynucleotide in a manner that precludes subsequent release of the DNA. Thus, transcription from the particle-bound DNA can occur, but the frequency with which it is released to become integrated into the genome is greatly reduced. Such methods include the use of particles coated with polyethylenimine (PEI; Sigma #P3143).

In other aspects, the polynucleotide may be introduced into plants by contacting plants with a virus or viral nucleic acids. Generally, such methods involve incorporating a nucleotide construct within a viral DNA or RNA molecule. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931 and Porta, et al., (1996) Molecular Biotechnology 5:209-221, herein incorporated by reference in their entirety.

The cells that have been transformed may be grown into plants in accordance with conventional ways. See, for example, McCormick, et al., (1986) Plant Cell Reports 5:81-84, herein incorporated by reference in its entirety. These plants may then be grown, and either pollinated with the same transformed strain or different strains, and the resulting progeny having expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present disclosure provides transformed seed (also referred to as “transgenic seed”) having a nucleotide construct, for example, an expression cassette, stably incorporated into its genome.

There are a variety of methods for the regeneration of plants from plant tissue. The particular method of regeneration will depend on the starting plant tissue and the particular plant species to be regenerated. The regeneration, development and cultivation of plants from single plant protoplast transformants or from various transformed explants is well known in the art (Weissbach and Weissbach, (1988) In: Methods for Plant Molecular Biology, (Eds.), Academic Press, Inc., San Diego, Calif., herein incorporated by reference in its entirety). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the aspects containing a desired polynucleotide is cultivated using methods well known to one skilled in the art.

Methods are known in the art for the targeted insertion of a polynucleotide at a specific location in the plant genome. The insertion of the polynucleotide at a desired genomic location is achieved using a site-specific recombination system. See, for example, U.S. Pat. No. 9,222,098 B2, U.S. Pat. No. 7,223,601 B2, U.S. Pat. No. 7,179,599 B2, and U.S. Pat. No. 6,911,575 B1, all of which are herein incorporated by reference in their entirety. Briefly, a polynucleotide of interest, flanked by two non-identical recombination sites, can be contained in a T-DNA transfer cassette. The T-DNA transfer cassette is introduced into a plant having stably incorporated into its genome a target site which is flanked by two non-identical recombination sites that correspond to the sites of the transfer cassette. An appropriate recombinase is provided, and the transfer cassette is integrated at the target site. The polynucleotide of interest is thereby integrated at a specific chromosomal position in the plant genome.

In an aspect, the disclosed methods can be used to introduce into vegetative plant organs and their composite tissues including but are not limited to leaf explants, leaf primordia, stipule, cotyledons, cotyledonary nodes, mesocotyl, stem explants, primary roots, lateral secondary roots, root segments, buds, and meristems, including but not limited to apical meristems, root meristems, secondary meristems, axillary meristems, and floral meristems, with increased efficiency and speed polynucleotides useful to target a specific site for modification in the genome of a plant. Site specific modifications that can be introduced with the disclosed methods include those produced using any method for introducing site specific modification, including, but not limited to, through the use of gene repair oligonucleotides (e.g. US Publication 2013/0019349), or through the use of double-stranded break technologies such as TALENs, meganucleases, zinc finger nucleases, CRISPR-Cas, and the like. For example, the disclosed methods can be used to introduce a CRISPR-Cas system into a plant cell or plant, for the purpose of genome modification of a target sequence in the genome of a plant or plant cell, for selecting plants, for deleting a base or a sequence, for gene editing, and for inserting a polynucleotide of interest into the genome of a plant or plant cell. Thus, the disclosed methods can be used together with a CRISPR-Cas system to provide for an effective system for modifying or altering target sites and nucleotides of interest within the genome of a plant, plant cell or seed. The Cas endonuclease gene is a plant optimized Cas9 endonuclease, wherein the plant optimized Cas9 endonuclease is capable of binding to and creating a double strand break in a genomic target sequence the plant genome.

The Cas endonuclease is guided by the guide nucleotide to recognize and optionally introduce a double strand break at a specific target site into the genome of a cell. The CRISPR-Cas system provides for an effective system for modifying target sites within the genome of a plant, plant cell or seed. Further provided are methods and compositions employing a guide polynucleotide/Cas endonuclease system to provide an effective system for modifying target sites within the genome of a cell and for editing a nucleotide sequence in the genome of a cell. Once a genomic target site is identified, a variety of methods can be employed to further modify the target sites such that they contain a variety of polynucleotides of interest. The disclosed compositions and methods can be used to introduce a CRISPR-Cas system for editing a nucleotide sequence in the genome of a cell. The nucleotide sequence to be edited (the nucleotide sequence of interest) can be located within or outside a target site that is recognized by a Cas endonuclease.

CRISPR loci (Clustered Regularly Interspaced Short Palindromic Repeats) (also known as SPIDRs-SPacer Interspersed Direct Repeats) constitute a family of recently described DNA loci. CRISPR loci consist of short and highly conserved DNA repeats (typically 24 to 40 bp, repeated from 1 to 140 times—also referred to as CRISPR-repeats) which are partially palindromic. The repeated sequences (usually specific to a species) are interspaced by variable sequences of constant length (typically 20 to 58 by depending on the CRISPR locus (WO2007/025097 published Mar. 1, 2007).

Cas gene includes a gene that is generally coupled, associated or close to or in the vicinity of flanking CRISPR loci. The terms “Cas gene” and “CRISPR-associated (Cas) gene” are used interchangeably herein.

In another aspect, the Cas endonuclease gene is operably linked to a SV40 nuclear targeting signal upstream of the Cas codon region and a bipartite VirD2 nuclear localization signal (Tinland et al. (1992) Proc. Natl. Acad. Sci. USA 89:7442-6) downstream of the Cas codon region.

As related to the Cas endonuclease, the terms “functional fragment,” “fragment that is functionally equivalent,” and “functionally equivalent fragment” are used interchangeably herein. These terms refer to a portion or subsequence of the Cas endonuclease sequence in which the ability to create a double-strand break is retained.

As related to the Cas endonuclease, the terms “functional variant,” “variant that is functionally equivalent” and “functionally equivalent variant” are used interchangeably herein. These terms refer to a variant of the Cas endonuclease in which the ability to create a double-strand break is retained. Fragments and variants can be obtained via methods such as site-directed mutagenesis and synthetic construction.

In an aspect, the Cas endonuclease gene is a plant codon optimized Streptococcus pyogenes Cas9 gene that can recognize any genomic sequence of the form N(12-30)NGG which can in principle be targeted.

Endonucleases are enzymes that cleave the phosphodiester bond within a polynucleotide chain and include restriction endonucleases that cleave DNA at specific sites without damaging the bases. Restriction endonucleases include Type I, Type II, Type III, and Type IV endonucleases, which further include subtypes. In the Type I and Type III systems, both the methylase and restriction activities are contained in a single complex. Endonucleases also include meganucleases, also known as homing endonucleases (HEases), which like restriction endonucleases, bind and cut at a specific recognition site, however the recognition sites for meganucleases are typically longer, about 18 bp or more (Patent application PCT/US 12/30061 filed on Mar. 22, 2012). Meganucleases have been classified into four families based on conserved sequence motifs. These motifs participate in the coordination of metal ions and hydrolysis of phosphodiester bonds. Meganucleases are notable for their long recognition sites, and for tolerating some sequence polymorphisms in their DNA substrates. The naming convention for meganuclease is similar to the convention for other restriction endonuclease. Meganucleases are also characterized by prefix F-, I-, or PI- for enzymes encoded by free-standing ORFs, introns, and inteins, respectively. One step in the recombination process involves polynucleotide cleavage at or near the recognition site. This cleaving activity can be used to produce a double-strand break. For reviews of site-specific recombinases and their recognition sites, see, Sauer (1994) Curr Op Biotechnol 5:521-7; and Sadowski (1993) FASEB 7:760-7. In some examples the recombinase is from the Integrase or Resolvase families. TAL effector nucleases are a new class of sequence-specific nucleases that can be used to make double-strand breaks at specific target sequences in the genome of a plant or other organism. (Miller, et al. (2011) Nature Biotechnology 29:143-148). Zinc finger nucleases (ZFNs) are engineered double-strand break inducing agents comprised of a zinc finger DNA binding domain and a double-strand-break-inducing agent domain. Recognition site specificity is conferred by the zinc finger domain, which typically comprising two, three, or four zinc fingers, for example having a C2H2 structure, however other zinc finger structures are known and have been engineered. Zinc finger domains are amenable for designing polypeptides which specifically bind a selected polynucleotide recognition sequence. ZFNs include an engineered DNA-binding zinc finger domain linked to a nonspecific endonuclease domain, for example nuclease domain from a Type Ms endonuclease such as Fokl. Additional functionalities can be fused to the zinc-finger binding domain, including transcriptional activator domains, transcription repressor domains, and methylases. In some examples, dimerization of nuclease domain is required for cleavage activity. Each zinc finger recognizes three consecutive base pairs in the target DNA. For example, a 3-finger domain recognized a sequence of 9 contiguous nucleotides, with a dimerization requirement of the nuclease, two sets of zinc finger triplets are used to bind an 18-nucleotide recognition sequence.

A “Dead-CAS9” (dCAS9) as used herein, is used to supply a transcriptional repressor domain. The dCAS9 has been mutated so that can no longer cut DNA. The dCASO can still bind when guided to a sequence by the gRNA and can also be fused to repressor elements. The dCAS9 fused to the repressor element, as described herein, is abbreviated to dCAS9˜REP, where the repressor element (REP) can be any of the known repressor motifs that have been characterized in plants. An expressed guide RNA (gRNA) binds to the dCAS9˜REP protein and targets the binding of the dCAS9-REP fusion protein to a specific predetermined nucleotide sequence within a promoter (a promoter within the T-DNA). For example, if this is expressed beyond—the border using a ZM-UBI PRO::dCAS9˜REP::PINII TERM cassette along with a U6-POL PRO::gRNA::U6 TERM cassette and the gRNA is designed to guide the dCAS9-REP protein to bind the SB-UBI promoter in the expression cassette SB-UBI PRO::moPAT::PINII TERM within the T-DNA, any event that has integrated the beyond-the-border sequence would be bialaphos sensitive. Transgenic events that integrate only the T-DNA would express moPAT and be bialaphos resistant. The advantage of using a dCAS9 protein fused to a repressor (as opposed to a TETR or ESR) is the ability to target these repressors to any promoter within the T-DNA. TETR and ESR are restricted to cognate operator binding sequences. Alternatively, a synthetic Zinc-Finger Nuclease fused to a repressor domain can be used in place of the gRNA and dCAS9˜REP (Urritia et al., 2003, Genome Biol. 4:231) as described above.

The type II CRISPR/Cas system from bacteria employs a crRNA and tracrRNA to guide the Cas endonuclease to its DNA target. The crRNA (CRISPR RNA) contains the region complementary to one strand of the double strand DNA target and base pairs with the tracrRNA (trans-activating CRISPR RNA) forming a RNA duplex that directs the Cas endonuclease to cleave the DNA target. As used herein, the term “guide nucleotide” relates to a synthetic fusion of two RNA molecules, a crRNA (CRISPR RNA) comprising a variable targeting domain, and a tracrRNA. In an aspect, the guide nucleotide comprises a variable targeting domain of 12 to 30 nucleotide sequences and a RNA fragment that can interact with a Cas endonuclease.

As used herein, the term “guide polynucleotide” relates to a polynucleotide sequence that can form a complex with a Cas endonuclease and enables the Cas endonuclease to recognize and optionally cleave a DNA target site. The guide polynucleotide can be a single molecule or a double molecule. The guide polynucleotide sequence can be a RNA sequence, a DNA sequence, or a combination thereof (a RNA-DNA combination sequence). Optionally, the guide polynucleotide can comprise at least one nucleotide, phosphodiester bond or linkage modification such as, but not limited, to Locked Nucleic Acid (LNA), 5-methyl dC, 2,6-Diaminopurine, 2′-Fluoro A, 2′-Fluoro U, 2′-O-Methyl RNA, phosphorothioate bond, linkage to a cholesterol molecule, linkage to a polyethylene glycol molecule, linkage to a spacer 18 (hexaethylene glycol chain) molecule, or 5′ to 3′ covalent linkage resulting in circularization. A guide polynucleotide that solely comprises ribonucleic acids is also referred to as a “guide nucleotide”.

Nucleotide sequence modification of the guide polynucleotide, VT domain and/or CER domain can be selected from, but not limited to, the group consisting of a 5′ cap, a 3′ polyadenylated tail, a riboswitch sequence, a stability control sequence, a sequence that forms a dsRNA duplex, a modification or sequence that targets the guide poly nucleotide to a subcellular location, a modification or sequence that provides for tracking, a modification or sequence that provides a binding site for proteins, a Locked Nucleic Acid (LNA), a 5-methyl dC nucleotide, a 2,6-Diaminopurine nucleotide, a 2′-Fluoro A nucleotide, a 2′-Fluoro U nucleotide; a 2′-O-Methyl RNA nucleotide, a phosphorothioate bond, linkage to a cholesterol molecule, linkage to a polyethylene glycol molecule, linkage to a spacer 18 molecule, a 5′ to 3′ covalent linkage, or any combination thereof. These modifications can result in at least one additional beneficial feature, wherein the additional beneficial feature is selected from the group of a modified or regulated stability, a subcellular targeting, tracking, a fluorescent label, a binding site for a protein or protein complex, modified binding affinity to complementary target sequence, modified resistance to cellular degradation, and increased cellular permeability.

In an aspect, the guide nucleotide and Cas endonuclease are capable of forming a complex that enables the Cas endonuclease to introduce a double strand break at a DNA target site.

In an aspect of the methods of the disclosure the variable target domain is 12, 13, 14, 15, 16, 17, 18, 19, 20, 21, 22, 23, 24, 25, 26, 27, 28, 29 or 30 nucleotides in length.

In an aspect of the methods of the disclosure, the guide nucleotide comprises a cRNA (or cRNA fragment) and a tracrRNA (or tracrRNA fragment) of the type II CRISPR/Cas system that can form a complex with a type II Cas endonuclease, wherein the guide nucleotide Cas endonuclease complex can direct the Cas endonuclease to a plant genomic target site, enabling the Cas endonuclease to introduce a double strand break into the genomic target site. The guide nucleotide can be introduced into a plant or plant cell directly using any method known in the art such as, but not limited to, particle bombardment or topical applications.

In an aspect, the guide nucleotide can be introduced indirectly by introducing a recombinant DNA molecule comprising the corresponding guide DNA sequence operably linked to a plant specific promoter that is capable of transcribing the guide nucleotide in the plant cell. The term “corresponding guide DNA” includes a DNA molecule that is identical to the RNA molecule but has a “T” substituted for each “U” of the RNA molecule.

In an aspect, the guide nucleotide is introduced via particle bombardment or using the disclosed methods and compositions for Agrobacterium transformation of a recombinant DNA construct comprising the corresponding guide DNA operably linked to a plant U6 polymerase III promoter.

In an aspect, the RNA that guides the RNA Cas9 endonuclease complex, is a duplexed RNA comprising a duplex crRNA-tracrRNA. One advantage of using a guide nucleotide versus a duplexed crRNA-tracrRNA is that only one expression cassette needs to be made to express the fused guide nucleotide.

The terms “target site,” “target sequence,” “target DNA,” “target locus,” “genomic target site,” “genomic target sequence,” and “genomic target locus” are used interchangeably herein and refer to a polynucleotide sequence in the genome (including choloroplastic and mitochondrial DNA) of a plant cell at which a double-strand break is induced in the plant cell genome by a Cas endonuclease. The target site can be an endogenous site in the plant genome, or alternatively, the target site can be heterologous to the plant and thereby not be naturally occurring in the genome, or the target site can be found in a heterologous genomic location compared to where it occurs in nature.

As used herein, terms “endogenous target sequence” and “native target sequence” are used interchangeably herein to refer to a target sequence that is endogenous or native to the genome of a plant and is at the endogenous or native position of that target sequence in the genome of the plant. In an aspect, the target site can be similar to a DNA recognition site or target site that is specifically recognized and/or bound by a double-strand break inducing agent such as a LIG3-4 endonuclease (US patent publication 2009/0133152 A1 (published May 21, 2009) or a MS26++ meganuclease (U.S. patent application Ser. No. 13/526,912 filed Jun. 19, 2012).

An “artificial target site” or “artificial target sequence” are used interchangeably herein and refer to a target sequence that has been introduced into the genome of a plant. Such an artificial target sequence can be identical in sequence to an endogenous or native target sequence in the genome of a plant but be located in a different position (i.e., a non-endogenous or non-native position) in the genome of a plant.

An “altered target site,” “altered target sequence” “modified target site,” and “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, or (iv) any combination of (i)-(iii).

In an aspect, the disclosed methods can be used to introduce into plants polynucleotides useful for gene suppression of a target gene in a plant. Reduction of the activity of specific genes (also known as gene silencing, or gene suppression) is desirable for several aspects of genetic engineering in plants. Many techniques for gene silencing are well known to one of skill in the art, including but not limited to antisense technology.

In an aspect, the disclosed methods can be used to introduce into plants polynucleotides useful for the targeted integration of nucleotide sequences into a plant. For example, the disclosed methods can be used to introduce T-DNA expression cassettes comprising nucleotide sequences of interest flanked by non-identical recombination sites are used to transform a plant comprising a target site. In an aspect, the target site contains at least a set of non-identical recombination sites corresponding to those on the T-DNA expression cassette. The exchange of the nucleotide sequences flanked by the recombination sites is affected by a recombinase. Thus, the disclosed methods can be used for the introduction of T-DNA expression cassettes for targeted integration of nucleotide sequences, wherein the T-DNA expression cassettes which are flanked by non-identical recombination sites recognized by a recombinase that recognizes and implements recombination at the nonidentical recombination sites. Accordingly, the disclosed methods and composition can be used to improve efficiency and speed of development of plants containing non-identical recombination sites.

Thus, the disclosed methods can further comprise methods for the directional, targeted integration of exogenous nucleotides into a transformed plant. In an aspect, the disclosed methods use novel recombination sites in a gene targeting system which facilitates directional targeting of desired genes and nucleotide sequences into corresponding recombination sites previously introduced into the target plant genome.

In an aspect, a nucleotide sequence flanked by two non-identical recombination sites is introduced into one or more cells of an explant derived from the target organism's genome establishing a target site for insertion of nucleotide sequences of interest. Once a stable plant or cultured tissue is established a second construct, or nucleotide sequence of interest, flanked by corresponding recombination sites as those flanking the target site, is introduced into the stably transformed plant or tissues in the presence of a recombinase protein. This process results in exchange of the nucleotide sequences between the non-identical recombination sites of the target site and the T-DNA expression cassette.

It is recognized that the transformed plant prepared in this manner may comprise multiple target sites; i. e., sets of non-identical recombination sites. In this manner, multiple manipulations of the target site in the transformed plant are available. By target site in the transformed plant is intended a DNA sequence that has been inserted into the transformed plant's genome and comprises non-identical recombination sites.

Examples of recombination sites for use in the disclosed method are known. The two-micron plasmid found in most naturally occurring strains of Saccharomyces cerevisiae, encodes a site-specific recombinase that promotes an inversion of the DNA between two inverted repeats. This inversion plays a central role in plasmid copy-number amplification.

The protein, designated FLP protein, catalyzes site-specific recombination events. The minimal recombination site (FRT) has been defined and contains two inverted 13-base pair (bp) repeats surrounding an asymmetric 8-bp spacer. The FLP protein cleaves the site at the junctions of the repeats and the spacer and is covalently linked to the DNA via a 3′phosphate. Site specific recombinases like FLP cleave and religate DNA at specific target sequences, resulting in a precisely defined recombination between two identical sites. To function, the system needs the recombination sites and the recombinase. No auxiliary factors are needed. Thus, the entire system can be inserted into and function in plant cells. The yeast FLP\FRT site specific recombination system has been shown to function in plants. To date, the system has been utilized for excision of unwanted DNA. See, Lyznik et at. (1993) Nucleic Acid Res. 21: 969-975. In contrast, the present disclosure utilizes non-identical FRTs for the exchange, targeting, arrangement, insertion and control of expression of nucleotide sequences in the plant genome.

In an aspect, a transformed organism of interest, such as an explant from a plant, containing a target site integrated into its genome is needed. The target site is characterized by being flanked by non-identical recombination sites. A targeting cassette is additionally required containing a nucleotide sequence flanked by corresponding non-identical recombination sites as those sites contained in the target site of the transformed organism. A recombinase which recognizes the non-identical recombination sites and catalyzes site-specific recombination is required.

It is recognized that the recombinase can be provided by any means known in the art. That is, it can be provided in the organism or plant cell by transforming the organism with an expression cassette capable of expressing the recombinase in the organism, by transient expression, or by providing messenger RNA (mRNA) for the recombinase or the recombinase protein.

By “non-identical recombination sites” it is intended that the flanking recombination sites are not identical in sequence and will not recombine or recombination between the sites will be minimal. That is, one flanking recombination site may be a FRT site where the second recombination site may be a mutated FRT site. The non-identical recombination sites used in the methods of the present disclosure prevent or greatly suppress recombination between the two flanking recombination sites and excision of the nucleotide sequence contained therein. Accordingly, it is recognized that any suitable non-identical recombination sites may be utilized in the present disclosure, including FRT and mutant FRT sites, FRT and lox sites, lox and mutant lox sites, as well as other recombination sites known in the art.

By suitable non-identical recombination site implies that in the presence of active recombinase, excision of sequences between two non-identical recombination sites occurs, if at all, with an efficiency considerably lower than the recombinationally-mediated exchange targeting arrangement of nucleotide sequences into the plant genome. Thus, suitable non-identical sites for use in the present disclosure include those sites where the efficiency of recombination between the sites is low; for example, where the efficiency is less than about 30 to about 50%, preferably less than about 10 to about 30%, more preferably less than about 5 to about 10%.

As noted above, the recombination sites in the targeting cassette correspond to those in the target site of the transformed plant. That is, if the target site of the transformed plant contains flanking non-identical recombination sites of FRT and a mutant FRT, the targeting cassette will contain the same FRT and mutant FRT non-identical recombination sites.

It is furthermore recognized that the recombinase, which is used in the disclosed methods, will depend upon the recombination sites in the target site of the transformed plant and the targeting cassette. That is, if FRT sites are utilized, the FLP recombinase will be needed. In the same manner, where lox sites are utilized, the Cre recombinase is required. If the non-identical recombination sites comprise both a FRT and a lox site, both the FLP and Cre recombinase will be required in the plant cell.

The FLP recombinase is a protein which catalyzes a site-specific reaction that is involved in amplifying the copy number of the two-micron plasmid of S. cerevisiae during DNA replication. FLP protein has been cloned and expressed. See, for example, Cox (1993) Proc. Natl. Acad. Sci. U.S.A 80: 4223-4227. The FLP recombinase for use in the present disclosure may be that derived from the genus Saccharomyces. It may be preferable to synthesize the recombinase using plant preferred codons for optimum expression in a plant of interest. See, for example, U.S. application Ser. No. 08/972,258 filed Nov. 18, 1997, entitled “Novel Nucleic Acid Sequence Encoding FLP Recombinase,” herein incorporated by reference.

The bacteriophage recombinase Cre catalyzes site-specific recombination between two lox sites. The Cre recombinase is known in the art. See, for example, Guo et al. (1997) Nature 389: 40-46; Abremski et al. (1984) J. Biol. Chem. 259: 1509-1514; Chen et al. (1996) Somat. Cell Mol. Genet. 22: 477-488; and Shaikh et al. (1977) J. Biol. Chem. 272: 5695-5702. All of which are herein incorporated by reference. Such Cre sequence may also be synthesized using plant preferred codons.

Where appropriate, the nucleotide sequences to be inserted in the plant genome may be optimized for increased expression in the transformed plant. Where mammalian, yeast, or bacterial genes are used in the present disclosure, they can be synthesized using plant preferred codons for improved expression. It is recognized that for expression in monocots, dicot genes can also be synthesized using monocot preferred codons. Methods are available in the art for synthesizing plant preferred genes. See, for example, U. S. Pat. Nos. 5,380,831, 5,436,391, and Murray et al. (1989) Nucleic Acids Res. 17: 477-498, herein incorporated by reference. The plant preferred codons may be determined from the codons utilized more frequently in the proteins expressed in the plant of interest. It is recognized that monocot or dicot preferred sequences may be constructed as well as plant preferred sequences for particular plant species. See, for example, EPA 0359472; EPA 0385962; WO 91/16432; Perlak et al. (1991) Proc. Natl. Acad. Sci. USA, 88: 3324-3328; and Murray et al. (1989) Nucleic Acids Research, 17: 477-498. U.S. Pat. Nos. 5,380,831; 5,436,391; and the like, herein incorporated by reference. It is further recognized that all or any part of the gene sequence may be optimized or synthetic. That is, fully optimized or partially optimized sequences may also be used.

Additional sequence modifications are known to enhance gene expression in a cellular host and can be used in the present disclosure. These include elimination of sequences encoding spurious polyadenylation signals, exon-intron splice site signals, transposon-like repeats, and other such well-characterized sequences, which may be deleterious to gene expression. The G-C content of the sequence may be adjusted to levels average for a given cellular host, as calculated by reference to known genes expressed in the host cell. When possible, the sequence is modified to avoid predicted hairpin secondary RNA structures.

The present disclosure also encompasses novel FLP recombination target sites (FRT). The FRT has been identified as a minimal sequence comprising two 13 base pair repeats, separated by an eight (8) base spacer. The nucleotides in the spacer region can be replaced with a combination of nucleotides, so long as the two 13-base repeats are separated by eight nucleotides. It appears that the actual nucleotide sequence of the spacer is not critical; however, for the practice of the present disclosure, some substitutions of nucleotides in the space region may work better than others. The eight-base pair spacer is involved in DNA-DNA pairing during strand exchange. The asymmetry of the region determines the direction of site alignment in the recombination event, which will subsequently lead to either inversion or excision. As indicated above, most of the spacer can be mutated without a loss of function. See, for example, Schlake and Bode (1994) Biochemistry 33: 12746-12751, herein incorporated by reference.

Novel FRT mutant sites can be used in the practice of the disclosed methods. Such mutant sites may be constructed by PCR-based mutagenesis. Although mutant FRT sites are known (see SEQ ID Nos 2, 3, 4 and 5 of WO1999/025821), it is recognized that other mutant FRT sites may be used in the practice of the present disclosure. The present disclosure is not restricted to the use of a particular FRT or recombination site, but rather that non-identical recombination sites or FRT sites can be utilized for targeted insertion and expression of nucleotide sequences in a plant genome. Thus, other mutant FRT sites can be constructed and utilized based upon the present disclosure.

As discussed above, bringing genomic DNA containing a target site with non-identical recombination sites together with a vector containing a T-DNA expression cassette with corresponding non-identical recombination sites, in the presence of the recombinase, results in recombination. The nucleotide sequence of the T-DNA expression cassette located between the flanking recombination sites is exchanged with the nucleotide sequence of the target site located between the flanking recombination sites. In this manner, nucleotide sequences of interest may be precisely incorporated into the genome of the host.

It is recognized that many variations of the present disclosure can be practiced. For example, target sites can be constructed having multiple non-identical recombination sites. Thus, multiple genes or nucleotide sequences can be stacked or ordered at precise locations in the plant genome. Likewise, once a target site has been established within the genome, additional recombination sites may be introduced by incorporating such sites within the nucleotide sequence of the T-DNA expression cassette and the transfer of the sites to the target sequence. Thus, once a target site has been established, it is possible to subsequently add sites, or alter sites through recombination.

Another variation includes providing a promoter or transcription initiation region operably linked with the target site in an organism. Preferably, the promoter will be 5′ to the first recombination site. By transforming the organism with a T-DNA expression cassette comprising a coding region, expression of the coding region will occur upon integration of the T-DNA expression cassette into the target site. This aspect provides for a method to select transformed cells, particularly plant cells, by providing a selectable marker sequence as the coding sequence.

Other advantages of the present system include the ability to reduce the complexity of integration of transgenes or transferred DNA in an organism by utilizing T-DNA expression cassettes as discussed above and selecting organisms with simple integration patterns. In the same manner, preferred sites within the genome can be identified by comparing several transformation events. A preferred site within the genome includes one that does not disrupt expression of essential sequences and provides for adequate expression of the transgene sequence.

The disclosed methods also provide for means to combine multiple expression cassettes at one location within the genome. Recombination sites may be added or deleted at target sites within the genome.

Any means known in the art for bringing the three components of the system together may be used in the present disclosure. For example, a plant can be stably transformed to harbor the target site in its genome. The recombinase may be transiently expressed or provided. Alternatively, a nucleotide sequence capable of expressing the recombinase may be stably integrated into the genome of the plant. In the presence of the corresponding target site and the recombinase, the T-DNA expression cassette, flanked by corresponding non-identical recombination sites, is inserted into the transformed plant's genome.

Alternatively, the components of the system may be brought together by sexually crossing transformed plants. In this aspect, a transformed plant, parent one, containing a target site integrated in its genome can be sexually crossed with a second plant, parent two, that has been genetically transformed with a T-DNA expression cassette containing flanking non-identical recombination sites, which correspond to those in plant one. Either plant one or plant two contains within its genome a nucleotide sequence expressing recombinase. The recombinase may be under the control of a constitutive or inducible promoter. In this manner, expression of recombinase and subsequent activity at the recombination sites can be controlled.

The disclosed methods are useful in targeting the integration of transferred nucleotide sequences to a specific chromosomal site. The nucleotide sequence may encode any nucleotide sequence of interest. Particular genes of interest include those which provide a readily analyzable functional feature to the host cell and/or organism, such as marker genes, as well as other genes that alter the phenotype of the recipient cells, and the like. Thus, genes effecting plant growth, height, susceptibility to disease, insects, nutritional value, and the like may be utilized in the present disclosure. The nucleotide sequence also may encode an ‘antisense’ sequence to turn off or modify gene expression.

It is recognized that the nucleotide sequences will be utilized in a functional expression unit or T-DNA expression cassette. By functional expression unit or T-DNA expression cassette is intended, the nucleotide sequence of interest with a functional promoter, and in most instances a termination region. There are various ways to achieve the functional expression unit within the practice of the present disclosure. In one aspect of the present disclosure, the nucleic acid of interest is transferred or inserted into the genome as a functional expression unit.

Alternatively, the nucleotide sequence may be inserted into a site within the genome which is 3′ to a promoter region. In this latter instance, the insertion of the coding sequence 3′ to the promoter region is such that a functional expression unit is achieved upon integration. The T-DNA expression cassette will comprise a transcriptional initiation region, or promoter, operably linked to the nucleic acid encoding the peptide of interest. Such an expression cassette is provided with a plurality of restriction sites for insertion of the gene or genes of interest to be under the transcriptional regulation of the regulatory regions.

A summary of SEQ ID NOS: 1-198 useful in the methods of the disclosure is presented in Table 3.

TABLE 3
Summary of SEQ ID NOS: 1-198.

| | Polynucleotide | | |
|---|---|---|---|
| SEQ | (DNA) or | | |
| ID | Polypeptide | | |
| NO. | (PRT) | Name | Description |
| 1 | DNA | GM-HBSTART3 | Glycine max HBSTART3 |
| | | | (Homeodomain StAR-related lipid |
| | | | transfer3) promoter sequence |
| 2 | DNA | GM-HBSTART3 | Glycine max HBSTART3 |
| | | (TRUNCATED) | (Homeodomain StAR-related lipid |
| | | | transfer3) promoter sequence |
| | | | (TRUNCATED) |
| 3 | DNA | AT-ML1 | Arabidopsis thaliana ML1 (MERISTEM |
| | | | LAYER1) promoter sequence |
| 4 | DNA | GM-ML1-Like | Glycine max ML1-Like (MERISTEM |
| | | | LAYER1-Like) promoter sequence |
| 5 | DNA | GM-ML1-Like | Glycine max ML1-Like (MERISTEM |
| | | (TRUNCATED) | LAYER1-Like) promoter sequence |
| | | | (TRUNCATED) |
| 6 | DNA | ZM-HBSTART3 | Zea mays HB START3 (Homeodomain |
| | | | StAR-related lipid transfer3) promoter |
| | | | sequence |
| 7 | DNA | OS-HBSTART3 | Oryza sativa HB START3 |
| | | | (Homeodomain StAR-related lipid |
| | | | transfer3) promoter sequence |
| 8 | DNA | AT-PDF1 | Arabidopsis thaliana PDF1 |
| | | | (PROTODERMAL FACTOR1) |
| | | | promoter sequence |
| 9 | DNA | GM-PDF1 | Glycine max PDF1 (PROTODERMAL |
| | | | FACTOR1) promoter sequence |
| 10 | DNA | GM-PDF1 | Glycine max PDF1 (PROTODERMAL |
| | | (TRUNCATED) | FACTOR1) promoter sequence |
| | | | (TRUNCATED) |
| 11 | DNA | SB-PDF1 | Sorghum bicolor PDF1 |
| | | | (PROTODERMAL FACTOR1) |
| | | | promoter sequence |
| 12 | DNA | OS-PDF1 | Oryza sativa PDF1 (PROTODERMAL |
| | | | FACTOR1) promoter sequence |
| 13 | DNA | OS-PDF1 | Oryza sativa PDF1 (PROTODERMAL |
| | | (TRUNCATED) | FACTOR1) promoter sequence |
| | | | (TRUNCATED) |
| 14 | DNA | PT-PDF 1 | Populus trichocarpa PDF1 |
| | | | (PROTODERMAL FACTOR1) |
| | | | promoter sequence |
| 15 | DNA | PT-PDF 1 | Populus trichocarpa PDF1 |
| | | (TRUNCATED) | (PROTODERMAL FACTOR1) |
| | | | promoter sequence (TRUNCATED) |
| 16 | DNA | SI-PDF 1 | Setaria italica PDF1 (PROTODERMAL |
| | | | FACTOR1) promoter sequence |
| 17 | DNA | SI-PDF 1 | Setaria italica PDF1 (PROTODERMAL |
| | | (TRUNCATED) | FACTOR1) promoter sequence |
| | | | (TRUNCATED) |
| 18 | DNA | AT-PDF2 | Arabidopsis thaliana PDF2 |
| | | | (PROTODERMAL FACTOR2) |
| | | | promoter sequence |
| 19 | DNA | GM-PDF2 | Glycine max PDF2 (PROTODERMAL |
| | | | FACTOR2) promoter sequence |
| 20 | DNA | GM-PDF2 | Glycine max PDF2 (PROTODERMAL |
| | | (TRUNCATED) | FACTOR2) promoter sequence |
| | | | (TRUNCATED) |
| 21 | DNA | ZM-GL1 | Zea mays GL1 (GLABROUS1) promoter |
| | | | sequence |
| 22 | DNA | AT-PDF2a | Arabidopsis thaliana PDF2a |
| | | | (PROTODERMAL FACTOR2a) |
| | | | promoter sequence |
| 23 | DNA | AT-PDF2a | Arabidopsis thaliana PDF2a |
| | | (TRUNCATED) | (PROTODERMAL FACTOR2a) |
| | | | promoter sequence (TRUNCATED) |
| 24 | DNA | GM-PDF2a | Glycine max PDF2a (PROTODERMAL |
| | | | FACTOR2a) promoter sequence |
| 25 | DNA | GM-PDF2a | Glycine max PDF2a (PROTODERMAL |
| | | (TRUNCATED) | FACTOR2a) promoter sequence |
| | | | (TRUNCATED) |
| 26 | DNA | OS-PDF2 | Oryza sativa PDF2 (PROTODERMAL |
| | | | FACTOR2) promoter sequence |
| 27 | DNA | OS-PDF2 | Oryza sativa PDF2 (PROTODERMAL |
| | | (TRUNCATED) | FACTOR2) promoter sequence |
| | | | (TRUNCATED) |
| 28 | DNA | PT-PDF2 | Populus trichocarpa PDF2 |
| | | | (PROTODERMAL FACTOR2) |
| | | | promoter sequence) |
| 29 | DNA | PT-PDF2 | Populus trichocarpa PDF2 |
| | | (TRUNCATED) | (PROTODERMAL FACTOR2) |
| | | | promoter sequence (TRUNCATED) |
| 30 | DNA | VV-PDF2 | Vitis vinifera PDF2 (PROTODERMAL |
| | | | FACTOR2) promoter sequence |
| 31 | DNA | VV-PDF2 | Vitis vinifera PDF2 (PROTODERMAL |
| | | (TRUNCATED) | FACTOR2) promoter sequence |
| | | | (TRUNCATED) |
| 32 | DNA | ZM-PDF2 | Zea mays PDF2 (PROTODERMAL |
| | | | FACTOR2) promoter sequence |
| 33 | DNA | SI-PDF2 | Setaria italica PDF2 (PROTODERMAL |
| | | | FACTOR2) promoter sequence |
| 34 | DNA | SI-PDF2 | Setaria italica PDF2 (PROTODERMAL |
| | | (TRUNCATED) | FACTOR2) promoter |
| | | | sequence(TRUNCATED) |
| 35 | DNA | VV-PDF2a | Vitis vinifera PDF2a (PROTODERMAL |
| | | | FACTOR2a) promoter sequence |
| 36 | DNA | PT-PDF2a | Populus trichocarpa PDF2a |
| | | | (PROTODERMAL FACTOR2a) |
| | | | promoter sequence |
| 37 | DNA | PT-PDF2a | Populus trichocarpa PDF2a |
| | | (TRUNCATED) | (PROTODERMAL FACTOR2a) |
| | | | promoter sequence (TRUNCATED) |
| 38 | DNA | MT-PDF2 | Medicago truncatula PDF2 |
| | | | (PROTODERMAL FACTOR2) |
| | | | promoter sequence |
| 39 | DNA | MT-PDF2 | Medicago truncatula PDF2 |
| | | (TRUNCATED) | (PROTODERMAL FACTOR2) |
| | | | promoter sequence (TRUNCATED) |
| 40 | DNA | AT-HDG2 | Arabidopsis thaliana HDG2 |
| | | | (HOMEODOMAIN GLABROUS2) |
| | | | promoter sequence |
| 41 | DNA | GM-HDG2 | Glycine max HDG2 (HOMEODOMAIN |
| | | | GLABROUS2) promoter sequence |
| 42 | DNA | GM-HDG2 | Glycine max HDG2 (HOMEODOMAIN |
| | | (TRUNCATED) | GLABROUS2) promoter sequence |
| | | | (TRUNCATED) |
| 43 | DNA | SB-HDG2 | Sorghum bicolor HDG2 |
| | | | (HOMEODOMAIN GLABROUS2) |
| | | | promoter sequence |
| 44 | DNA | SB-HDG2 | Sorghum bicolor HDG2 |
| | | (TRUNCATED) | (HOMEODOMAIN GLABROUS2) |
| | | | promoter sequence (TRUNCATED) |
| 45 | DNA | AT-CER6 | Arabidopsis thaliana CER6 |
| | | | (ECERIFERUM6) promoter sequence |
| 46 | DNA | AT-CER60 | Arabidopsis thaliana CER60 |
| | | | (ECERIFERUM60) promoter sequence |
| 47 | DNA | AT-CER60 | Arabidopsis thaliana CER60 |
| | | (TRUNCATED) | (ECERIFERUM60) promoter sequence |
| | | | (TRUNCATED) |
| 48 | DNA | GM-CER6 | Glycine max CER6 (ECERIFERUM6) |
| | | | promoter sequence |
| 49 | DNA | GM-CER6 | Glycine max CER6 (ECERIFERUM6) |
| | | (TRUNCATED) | promoter sequence (TRUNCATED) |
| 50 | DNA | PT-CER6 | Populus trichocarpa CER6 |
| | | | (ECERIFERUM6) promoter sequence |
| 51 | DNA | PT-CER6 | Populus trichocarpa CER6 |
| | | (TRUNCATED) | (ECERIFERUM6) promoter sequence |
| | | | (TRUNCATED) |
| 52 | DNA | VV-CER6 | Vitis vinifera CER6 (ECERIFERUM6) |
| | | | promoter sequence |
| 53 | DNA | VV-CER6 | Vitis vinifera CER6 (ECERIFERUM6) |
| | | (TRUNCATED) | promoter sequence (TRUNCATED) |
| 54 | DNA | SB-CER6 | Sorghum bicolor CER6 |
| | | | (ECERIFERUM6) promoter sequence |
| 55 | DNA | ZM-CER6 | Zea mays CER6 (ECERIFERUM6) |
| | | | promoter sequence |
| 56 | DNA | SI-CER6 | Setaria italica CER6 (ECERIFERUM6) |
| | | | promoter sequence |
| 57 | DNA | SI-CER6 | Setaria italica CER6 (ECERIFERUM6) |
| | | (TRUNCATED) | promoter sequence (TRUNCATED) |
| 58 | DNA | OS-CER6 | Oryza sativa CER6 (ECERIFERUM6) |
| | | | promoter sequence |
| 59 | DNA | OS-CER6 | Oryza sativa CER6 (ECERIFERUM6) |
| | | (TRUNCATED) | promoter sequence (TRUNCATED) |
| 60 | DNA | AT-WUS | Arabidopsis thaliana WUS coding |
| | | | sequence |
| 61 | PRT | AT-WUS | Arabidopsis thaliana WUS protein |
| | | | sequence |
| 62 | DNA | LJ-WUS | Lotus japonicus WUS coding sequence |
| 63 | PRT | LJ-WUS | Lotus japonicus WUS protein sequence |
| 64 | DNA | GM-WUS | Glycine max WUS coding sequence |
| 65 | PRT | GM-WUS | Glycine max WUS protein sequence |
| 66 | DNA | CS-WUS | Camelina sativa WUS coding sequence |
| 67 | PRT | CS-WUS | Camelina sativa WUS protein sequence |
| 68 | DNA | CR-WUS | Capsella rubella WUS coding sequence |
| 69 | PRT | CR-WUS | Capsella rubella WUS protein sequence |
| 70 | DNA | AA-WUS | Arabis alpina WUS coding sequence |
| 71 | PRT | AA-WUS | Arabis alpina WUS protein sequence |
| 72 | DNA | RS-WUS | Raphanus sativus WUS coding sequence |
| 73 | PRT | RS-WUS | Raphanus sativus WUS protein sequence |
| 74 | DNA | BN-WUS | Brassica napus WUS coding sequence |
| 75 | PRT | BN-WUS | Brassica napus WUS protein sequence |
| 76 | DNA | BO-WUS | Brassica oleracea var. oleracea WUS |
| | | | coding sequence |
| 77 | PRT | BO-WUS | Brassica oleracea var. oleracea WUS |
| | | | protein sequence |
| 78 | DNA | HA-WUS | Helianthus annuus WUS coding |
| | | | sequence |
| 79 | PRT | HA-WUS | Helianthus annuus WUS protein |
| | | | sequence |
| 80 | DNA | PT-WUS | Populus trichocarpa WUS coding |
| | | (POPTR-WUS) | sequence |
| 81 | PRT | PT-WUS | Populus trichocarpa WUS protein |
| | | (POPTR-WUS) | sequence |
| 82 | DNA | VV-WUS | Vitis vinifera WUS coding sequence |
| | | (VITVI-WUS) | |
| 83 | PRT | VV-WUS | Vitis vinifera WUS protein sequence |
| | | (VITVI-WUS) | |
| 84 | DNA | AT-WUS | Arabidopsis thaliana WUS coding |
| | | | sequence (soy optimized) |
| 85 | PRT | AT-WUS | Arabidopsis thaliana WUS protein |
| | | | sequence |
| 86 | DNA | LJ-WUS | Lotus japonicus WUS coding sequence |
| | | | (soy optimized) |
| 87 | PRT | LJ-WUS | Lotus japonicus WUS protein sequence |
| 88 | DNA | MT-WUS | Medicago truncatula WUS coding |
| | | | sequence (soy optimized) |
| 89 | PRT | MT-WUS | Medicago truncatula WUS protein |
| | | | sequence |
| 90 | DNA | PH-WUS | Petunia hybrida WUS coding sequence |
| | | (PETHY-WUS) | (soy optimized) |
| 91 | PRT | PH-WUS | Petunia hybrida WUS protein sequence |
| | | (PETHY-WUS) | |
| 92 | DNA | PV-WUS | Phaseolus vulgaris WUS coding |
| | | | sequence (soy optimized) |
| 93 | PRT | PV-WUS | Phaseolus vulgaris WUS protein |
| | | | sequence |
| 94 | DNA | ZM-WUS1 | Zea mays WUS1 coding sequence |
| 95 | PRT | ZM-WUS1 | Zea mays WUS1 protein sequence |
| 96 | DNA | ZM-WUS2 | Zea mays WUS2 coding sequence |
| 97 | PRT | ZM-WUS2 | Zea mays WUS2 protein sequence |
| 98 | DNA | ZM-WUS3 | Zea mays WUS3 coding sequence |
| 99 | PRT | ZM-WUS3 | Zea mays WUS3 protein sequence |
| 100 | DNA | ZM-WOX2A | Zea mays WOX2A coding sequence |
| 101 | PRT | ZM-WOX2A | Zea mays WOX2A protein sequence |
| 102 | DNA | ZM-WOX4 | Zea mays WOX4 coding sequence |
| 103 | PRT | ZM-WOX4 | Zea mays WOX4 protein sequence |
| 104 | DNA | ZM-WOX5A | Zea mays WOX5A coding sequence |
| 105 | PRT | ZM-WOX5A | Zea mays WOX5A protein sequence |
| 106 | DNA | ZM-WOX9 | Zea mays WOX9 coding sequence |
| 107 | PRT | ZM-WOX9 | Zea mays WOX9 protein sequence |
| 108 | DNA | GM-HBSTART2 | Glycine max HB START2 |
| | | | (Homeodomain StAR-related lipid |
| | | | transfer2) promoter sequence |
| 109 | DNA | GM-MATE1 | Glycine max MATE1 (Multi- |
| | | | antimicrobial extrusion protein 1) |
| | | | promoter sequence |
| 110 | DNA | GM-NED1 | Glycine max NED1 (NAD dependent |
| | | | epimerase/dehydratase1) promoter |
| | | | sequence |
| 111 | DNA | PHP80728 | Synthetic construct comprising the T- |
| | | | DNA (RB to LB) (RB + GM-UBQ |
| | | | PRO::GM-UBQ INTRON1::TAG- |
| | | | RFP::UBQ3 TERM + GM-SAMS |
| | | | PRO::GM-SAMS INTRON1::GM- |
| | | | HRA::GM-ALS TERM + LB) |
| 112 | DNA | PHP80730 | Synthetic construct comprising the T- |
| | | | DNA (RB to LB) (RB + GM-LTP3 |
| | | | PRO::AT-WUS::UBQ14 TERM + GM- |
| | | | UBQ PRO::GM-UBQ INTRON1::TAG- |
| | | | RFP::UBQ3 TERM + GM-SAMS |
| | | | PRO::GM-SAMS INTRON1::GM- |
| | | | HRA::GM-ALS TERM + LB) |
| 113 | DNA | PHP80734 | Synthetic construct comprising the T- |
| | | | DNA (RB to LB) (RB + GM- |
| | | | HBSTART2 PRO::AT-WUS::UBQ14 |
| | | | TERM + GM-UBQ PRO::GM-UBQ |
| | | | INTRON1::TAG-RFP::UBQ3 TERM + |
| | | | GM-SAMS PRO::GM-SAMS |
| | | | INTRON1::GM-HRA::GM-ALS |
| | | | TERM + LB) |
| 114 | DNA | PHP80736 | Synthetic construct comprising the T- |
| | | | DNA (RB to LB) (RB + GM-MATE1 |
| | | | PRO::AT-WUS::UBQ14 TERM + GM- |
| | | | UBQ PRO::GM-UBQ INTRON1::TAG- |
| | | | RFP::UBQ3 TERM + GM-SAMS |
| | | | PRO::GM-SAMS INTRON1::GM- |
| | | | HRA::GM-ALS TERM + LB) |
| 115 | DNA | PHP81060 | Synthetic construct comprising the T- |
| | | | DNA (RB to LB) (RB + GM-NED1 |
| | | | PRO::AT-WUS::UBQ14 TERM + GM- |
| | | | UBQ PRO::GM-UBQ INTRON1::TAG- |
| | | | RFP::UBQ3 TERM + GM-SAMS |
| | | | PRO::GM-SAMS INTRON1::GM- |
| | | | HRA::GM-ALS TERM + LB) |
| 116 | DNA | PHP81343 | Synthetic construct comprising the T- |
| | | | DNA (RB to LB) (RB + GM- |
| | | | HBSTART3 PRO::AT-WUS::UBQ14 |
| | | | TERM + GM-UBQ PRO::GM-UBQ |
| | | | INTRON1::TAG-RFP::UBQ3 TERM + |
| | | | GM-SAMS PRO::GM-SAMS |
| | | | INTRON1::GM-HRA::GM-ALS |
| | | | TERM + LB) |
| 117 | DNA | QC318 | GM-EF1A PRO::GM-EF1A |
| | | | INTRON1::ZS-YELLOW::NOS TERM + |
| | | | GM-SAMS PRO::GM-SAMS |
| | | | INTRON1::GM-ALS::GM-ALS TERM |
| 118 | DNA | pVER9662 | AT-UBIQ10 PRO::AT-UBIQ10 |
| | | | INTRON1::AT-WUS::UBQ14 TERM |
| 119 | DNA | UBIGMWUS | AT-UBIQ10 PRO::AT-UBIQ10 |
| | | | INTRON1::GM-WUS:: UBQ3 TERM |
| 120 | DNA | UBIMTWUS | AT-UBIQ10 PRO::AT-UBIQ10 |
| | | | INTRON1::MT-WUS:: UBQ3 TERM |
| 121 | DNA | UBILJWUS | AT-UBIQ10 PRO::AT-UBIQ10 |
| | | | INTRON1::11-WUS:: UBQ3 TERM |
| 122 | DNA | UBIPVWUS | AT-UBIQ10 PRO::AT-UBIQ10 |
| | | | INTRON1::PV-WUS:: UBQ3 TERM |
| 123 | DNA | UBIPHWUS | AT-UBIQ10 PRO::AT-UBIQ10 |
| | | | INTRON1::PH-WUS:: UBQ3 TERM |
| 124 | DNA | GM-LTP3 | Glycine max LTP3 (Lipid Transfer |
| | | | Protein3) promoter sequence |
| 125 | DNA | AT-ML1 | Arabidopsis thaliana ML1 (MERISTEM |
| | | (TRUNCATED) | LAYER1) promoter sequence |
| | | | (TRUNCATED) |
| 126 | DNA | AT-CER6 | Arabidopsis thaliana CER6 |
| | | (TRUNCATED1) | (ECERIFERUM6) promoter sequence |
| | | | (TRUNCATED) |
| 127 | DNA | GG-WUS | Gnetum gnemon WUS coding sequence |
| | | (GNEGN-WUS) | |
| 128 | PRT | GG-WUS | Gnetum gnemon WUS protein sequence |
| | | (GNEGN-WUS) | |
| 129 | DNA | MD-WUS | Malus domestica WUS coding sequence |
| | | (MALDO-WUS) | |
| 130 | PRT | MD-WUS | Malus domestica WUS protein sequence |
| | | (MALDO-WUS) | |
| 131 | DNA | ME-WUS | Manihot esculenta WUS coding |
| | | (MANES-WUS) | sequence |
| 132 | PRT | ME-WUS | Manihot esculenta WUS protein |
| | | (MANES-WUS) | sequence |
| 133 | DNA | KF-WUS | Kalanchoe fedtschenkoi WUS coding |
| | | (KALFE-WUS) | sequence |
| 134 | PRT | KF-WUS | Kalanchoe fedtschenkoi WUS protein |
| | | (KALFE-WUS) | sequence |
| 135 | DNA | GH-WUS | Gossypium hirsutum WUS coding |
| | | (GOSHI-WUS) | sequence |
| 136 | PRT | GH-WUS | Gossypium hirsutum WUS protein |
| | | (GOSHI-WUS) | sequence |
| 137 | DNA | ZOSMA-WUS | Zostera marina WUS coding sequence |
| 138 | PRT | ZOSMA-WUS | Zostera marina WUS protein sequence |
| 139 | DNA | AMBTR-WUS | Amborella trichopoda WUS coding |
| | | | sequence |
| 140 | PRT | AMBTR-WUS | Amborella trichopoda WUS protein |
| | | | sequence |
| 141 | DNA | AC-WUS | Aquilegia coerulea WUS coding |
| | | (AQUCO-WUS) | sequence |
| 142 | PRT | AC-WUS | Aquilegia coerulea WUS protein |
| | | (AQUCO-WUS) | sequence |
| 143 | DNA | AH-WUS | Amaranthus hypochondriacus WUS |
| | | (AMAHY-WUS) | coding sequence |
| 144 | PRT | AH-WUS | Amaranthus hypochondriacus WUS |
| | | (AMAHY-WUS) | protein sequence |
| 145 | DNA | CUCSA-WUS | Cucumis sativus WUS coding sequence |
| 146 | PRT | CUCSA-WUS | Cucumis sativus WUS protein sequence |
| 147 | DNA | PINTA-WUS | Pinus taeda WUS coding sequence |
| 148 | PRT | PINTA-WUS | Pinus taeda WUS protein sequence |
| 149 | DNA | AT-PDF1 | Arabidopsis thaliana PDF1 |
| | | (TRUNCATED) | (PROTODERMAL FACTOR1) |
| | | | promoter sequence (TRUNCATED) |
| 150 | DNA | AT-PDF2 | Arabidopsis thaliana PDF1 |
| | | (TRUNCATED) | (PROTODERMAL FACTOR1) |
| | | | promoter sequence (TRUNCATED) |
| 151 | DNA | AT-HDG2 | Arabidopsis thaliana HDG2 |
| | | (TRUNCATED) | (HOMEODOMAIN GLABROUS2) |
| | | | promoter sequence (TRUNCATED) |
| 152 | DNA | AT-ANL2 | Arabidopsis thaliana Anthocyanless2 |
| | | | promoter |
| 153 | DNA | RV021090 | Synthetic construct comprising the T- |
| | | | DNA (RB to LB): RB + CAMV35S |
| | | | PRO::HA-WUS::OS-T28 TERM + GM- |
| | | | UBQ PRO::GM-UBQ 5’ UTR::GM- |
| | | | UBQ INTRON1::ZS-YELLOW1 |
| | | | N1::NOS TERM + AT-UBIQ10 |
| | | | PRO::AT-UBIQ10 5’ UTR::AT-UBIQ10 |
| | | | INTRON1::CTP::SPCN::UBQ14 |
| | | | TERM + LB |
| 154 | DNA | RV026522 | T-DNA with GNEGN-WUS (GG - |
| | | | Gnetum gnemon) |
| | | | Synthetic construct comprising the T- |
| | | | DNA (RB to LB): RB + CAMV35S |
| | | | PRO:: GG-WUS::OS-T28 TERM + GM- |
| | | | UBQ PRO::GM-UBQ 5UTR::GM-UBQ |
| | | | INTRON1::ZS-YELLOW1 N1::NOS |
| | | | TERM + AT-UBIQ10 PRO::AT- |
| | | | UBIQ10 5UTR::AT-UBIQ10 |
| | | | INTRON1::CTP::SPCN::UBQ14 |
| | | | TERM + GM-EF1A2 PRO::GM-EF1A2 |
| | | | 5’ UTR::GM-EF1A2 INTRON1::DS- |
| | | | RED2::UBQ TERM + LB |
| 155 | DNA | RV026526 | T-DNA with VITVI-WUS (VV - Vitis |
| | | | vinifera) |
| | | | Synthetic construct comprising the T- |
| | | | DNA (RB to LB): RB + CAMV35S |
| | | | PRO::VV-WUS::OS-T28 TERM + GM- |
| | | | UBQ PRO::GM-UBQ 5’ UTR::GM- |
| | | | UBQ INTRON1::ZS-YELLOW1 |
| | | | N1::NOS TERM + AT-UBIQ10 |
| | | | PRO::AT-UBIQ10 5’ UTR::AT-UBIQ10 |
| | | | INTRON1::CTP::SPCN::UBQ14 |
| | | | TERM + GM-EF1A2 PRO::GM-EF1A2 |
| | | | 5’ UTR::GM-EF1A2 INTRON1::DS- |
| | | | RED2::UBQ TERM + LB |
| 156 | DNA | RV026534 | T-DNA with PETHY-WUS (PH - |
| | | | Petunia hybrida) |
| | | | Synthetic construct comprising the T- |
| | | | DNA (RB to LB): RB + CAMV35S |
| | | | PRO::PH-WUS::OS-T28 TERM + GM- |
| | | | UBQ PRO::GM-UBQ 5’ UTR::GM- |
| | | | UBQ INTRON1::ZS-YELLOW1 |
| | | | N1::NOS TERM + AT-UBIQ10 |
| | | | PRO::AT-UBIQ10 5’ UTR::AT-UBIQ10 |
| | | | INTRON1::CTP::SPCN::UBQ14 |
| | | | TERM + GM-EF1A2 PRO::GM-EF1A2 |
| | | | 5’ UTR::GM-EF1A2 INTRON1::DS- |
| | | | RED2::UBQ TERM + LB |
| 157 | DNA | RV026531 | T-DNA with MALDO-WUS (MD - |
| | | | Malus domestica) |
| | | | Synthetic construct comprising the T- |
| | | | DNA (RB to LB): RB + CAMV35S |
| | | | PRO::MD-WUS::OS-T28 TERM + GM- |
| | | | UBQ PRO::GM-UBQ 5UTR::GM-UBQ |
| | | | INTRON1::ZS-YELLOW1 N1::NOS |
| | | | TERM + AT-UBIQ10 PRO:: AT- |
| | | | UBIQ10 5UTR::AT-UBIQ10 |
| | | | INTRON1::CTP::SPCN::UBQ14 |
| | | | TERM + GM-EF1A2 PRO::GM-EF1A2 |
| | | | 5’ UTR::GM-EF1A2 INTRON1::DS- |
| | | | RED2::UBQ TERM + LB |
| 158 | DNA | RV026524 | T-DNA with MANES-WUS (ME - |
| | | | Manihot esculenta) |
| | | | Synthetic construct comprising the T- |
| | | | DNA (RB to LB): RB + CAMV35S |
| | | | PRO::ME-WUS::OS-T28 TERM + GM- |
| | | | UBQ PRO::GM-UBQ 5’ UTR::GM- |
| | | | UBQ INTRON1::ZS-YELLOW1 |
| | | | N1::NOS TERM + AT-UBIQ10 |
| | | | PRO::AT-UBIQ10 5’ UTR::AT-UBIQ10 |
| | | | INTRON1::CTP::SPCN::UBQ14 |
| | | | TERM + GM-EF1A2 PRO::GM-EF1A2 |
| | | | 5’ UTR::GM-EF1A2 INTRON1::DS- |
| | | | RED2::UBQ TERM + LB |
| 159 | DNA | RV026523 | T-DNA with KALFE-WUS (KF - |
| | | | Kalanchoe fedtschenkoi) |
| | | | Synthetic construct comprising the T- |
| | | | DNA (RB to LB): RB + CAMV35S |
| | | | PRO::KF-WUS::OS-T28 TERM +GM- |
| | | | UBQ PRO::GM-UBQ 5’ UTR::GM- |
| | | | UBQ INTRON1::ZS-YELLOW1 |
| | | | N1::NOS TERM + AT-UBIQ10 |
| | | | PRO::AT-UBIQ10 5’ UTR::AT-UBIQ10 |
| | | | INTRON1::CTP::SPCN::UBQ14 |
| | | | TERM + GM-EF1A2 PRO::GM-EF1A2 |
| | | | 5’ UTR::GM-EF1A2 INTRON1::DS- |
| | | | RED2::UBQ TERM + LB |
| 160 | DNA | RV026530 | T-DNA with GOSHI-WUS (GH - |
| | | | Gossypium hirsutum) |
| | | | Synthetic construct comprising the T- |
| | | | DNA (RB to LB): RB + CAMV35S |
| | | | PRO::GH-WUS::OS-T28 TERM + GM- |
| | | | UBQ PRO::GM-UBQ 5’ UTR::GM- |
| | | | UBQ INTRON1::ZS-YELLOW1 |
| | | | N1::NOS TERM + AT-UBIQ10 |
| | | | PRO::AT-UBIQ10 5’ UTR::AT- |
| | | | UBIQ10 |
| | | | INTRON1::CTP::SPCN::UBQ14 |
| | | | TERM + GM-EF1A2 PRO::GM-EF1A2 |
| | | | 5’ UTR::GM-EF1A2 INTRON1::DS- |
| | | | RED2::UBQ TERM + LB |
| 161 | DNA | RV026527 | T-DNA with ZOSMA-WUS (Zostera |
| | | | marina) |
| | | | Synthetic construct comprising the T- |
| | | | DNA (RB to LB): RB + CAMV35S |
| | | | PRO::ZOSMA-WUS::OS-T28 TERM + |
| | | | GM-UBQ PRO::GM-UBQ 5’ |
| | | | UTR::GM-UBQ INTRON1::ZS- |
| | | | YELLOW1 N1::NOS TERM + AT- |
| | | | UBIQ10 PRO::AT-UBIQ10 5’ |
| | | | UTR::AT-UBIQ10 |
| | | | INTRON1::CTP::SPCN::UBQ14 |
| | | | TERM + GM-EF1A2 PRO::GM-EF1A2 |
| | | | 5’ UTR::GM-EF1A2 INTRON1::DS- |
| | | | RED2::UBQ TERM + LB |
| 162 | DNA | RV026529 | T-DNA with AMB TR-WUS (Amborella |
| | | | trichopoda) |
| | | | Synthetic construct comprising the T- |
| | | | DNA (RB to LB): RB + CAMV35S |
| | | | PRO::AMBTR-WUS::OS-T28 TERM + |
| | | | GM-UBQ PRO::GM-UBQ 5’ |
| | | | UTR::GM-UBQ INTRON1::ZS- |
| | | | YELLOW1 N1::NOS TERM + AT- |
| | | | UBIQ10 PRO::AT-UBIQ10 5’ |
| | | | UTR::AT-UBIQ10 |
| | | | INTRON1::CTP::SPCN::UBQ14 |
| | | | TERM + GM-EF1A2 PRO::GM-EF1A2 |
| | | | 5’ UTR::GM-EF1A2 INTRON1::DS- |
| | | | RED2::UBQ TERM + LB |
| 163 | DNA | RV026528 | T-DNA with AQUCO-WUS (Aquilegia |
| | | | coerulea) |
| | | | Synthetic construct comprising the T- |
| | | | DNA (RB to LB): RB + CAMV35S |
| | | | PRO::AC-WUS::OS-T28 TERM + GM- |
| | | | UBQ PRO::GM-UBQ 5’ UTR::GM- |
| | | | UBQ INTRON1::ZS-YELLOW1 |
| | | | N1::NOS TERM + AT-UBIQ10 |
| | | | PRO::AT-UBIQ10 5’ UTR::AT-UBIQ10 |
| | | | INTRON1::CTP::SPCN::UBQ14 |
| | | | TERM + GM-EF1A2 PRO::GM-EF1A2 |
| | | | 5’ UTR::GM-EF1A2 INTRON1::DS- |
| | | | RED2::UBQ TERM + LB |
| 164 | DNA | RV026520 | T-DNA with POPTR-WUS (Populus |
| | | | trichocarpa) |
| | | | Synthetic construct comprising the T- |
| | | | DNA (RB to LB): RB + CAMV35S |
| | | | PRO::PT-WUS::OS-T28 TERM + GM- |
| | | | UBQ PRO::GM-UBQ 5’ UTR::GM- |
| | | | UBQ INTRON1::ZS-YELLOW1 |
| | | | N1::NOS TERM + AT-UBIQ10 |
| | | | PRO::AT-UBIQ10 5; UTR::AT-UBIQ10 |
| | | | INTRON1::CTP::SPCN::UBQ14 |
| | | | TERM + GM-EF1A2 PRO::GM-EF1A2 |
| | | | 5’ UTR::GM-EF1A2 INTRON1::DS- |
| | | | RED2::UBQ TERM + LB |
| 165 | DNA | RV026533 | T-DNA with AMAHY-WUS |
| | | | (Amaranthus hypochondriacus) |
| | | | Synthetic construct comprising the T- |
| | | | DNA (RB to LB): RB + CAMV35S |
| | | | PRO::AH-WUS::OS-T28 TERM + GM- |
| | | | UBQ PRO::GM-UBQ 5’ UTR::GM- |
| | | | UBQ INTRON1::ZS-YELLOW1 |
| | | | N1::NOS TERM + AT-UBIQ10 |
| | | | PRO::AT-UBIQ10 5’ UTR::AT-UBIQ10 |
| | | | INTRON1::CTP::SPCN::UBQ14 |
| | | | TERM + GM-EF1A2 PRO::GM-EF1A2 |
| | | | 5’ UTR::GM-EF1A2 INTRON1::DS- |
| | | | RED2::UBQ TERM + LB |
| 166 | DNA | RV026521 | T-DNA with CUCSA-WUS (Cucumis |
| | | | sativus) |
| | | | Synthetic construct comprising the T- |
| | | | DNA (RB to LB): RB + CAMV35S |
| | | | PRO::CS-WUS::OS-T28 TERM + GM- |
| | | | UBQ PRO::GM-UBQ 5’ UTR::GM- |
| | | | UBQ INTRON1::ZS-YELLOW1 |
| | | | N1::NOS TERM +AT-UBIQ10 |
| | | | PRO::AT-UBIQ10 5’ UTR::AT-UBIQ10 |
| | | | INTRON1::CTP::SPCN::UBQ14 |
| | | | TERM + GM-EF1A2 PRO::GM-EF1A2 |
| | | | 5’ UTR::GM-EF1A2 INTRON1::DS- |
| | | | RED2::UBQ TERM + LB |
| 167 | DNA | RV026525 | T-DNA with PINTA-WUS (Pinus taeda) |
| | | | Synthetic construct comprising the T- |
| | | | DNA (RB to LB): RB + CAMV35S |
| | | | PRO::PINTA-WUS::OS-T28 TERM + |
| | | | GM-UBQ PRO::GM-UBQ 5’ |
| | | | UTR::GM-UBQ INTRON1::ZS- |
| | | | YELLOW1 N1::NOS TERM + AT- |
| | | | UBIQ10 PRO::AT-UBIQ10 5’ |
| | | | UTR::AT-UBIQ10 |
| | | | INTRON1::CTP::SPCN::UBQ14 |
| | | | TERM + GM-EF1A2 PRO::GM-EF1A2 |
| | | | 5’ UTR::GM-EF1A2 INTRON1::DS- |
| | | | RED2::UBQ TERM + LB |
| 168 | DNA | RV022814 | T-DNA with NO WUS cassette |
| | | | Synthetic construct comprising the T- |
| | | | DNA (RB to LB): RB + CCDB + GM- |
| | | | UBQ PRO-V1::GM-UBQ 5’ UTR::GM- |
| | | | UBQ INTRON1::CTP::SPCN::UBQ14 |
| | | | TERM + GM-EF1A2 PRO:: GM-EF1A2 |
| | | | 5’ UTR:: GM-EF1A2 INTRON1:: GM- |
| | | | EF1A2 5’ UTR::DS-RED2::UBQ3 |
| | | | TERM + LB |
| 169 | DNA | RV027344 | T-DNA with AT-PDF2 PRO |
| | | | Synthetic construct comprising the T- |
| | | | DNA (RB to LB): RB + AT-PDF2 |
| | | | PRO::HA-WUS::OS-T28 TERM + GM- |
| | | | UBQ PRO::GM-UBQ 5’ UTR::GM- |
| | | | UBQ INTRON1::ZS-YELLOW1 |
| | | | N1::NOS TERM + AT-UBIQ10 |
| | | | PRO::AT-UBIQ10 5’ UTR::AT-UBIQ10 |
| | | | INTRON1::CTP::SPCN::UBQ14 |
| | | | TERM + GM-EF1A2 PRO::GM-EF1A2 |
| | | | 5’ UTR::GM-EF1A2 INTRON1::DS- |
| | | | RED2::UBQ TERM + LB |
| 170 | DNA | RV026532 | T-DNA for PRO TEST NEG-NO WUS |
| | | | Synthetic construct comprising the T- |
| | | | DNA (RB to LB): RB + GM-UBQ |
| | | | PRO-V1::GM-UBQ 5’ UTR::GM-UBQ |
| | | | INTRON1::ZS-YELLOW1 N1::NOS |
| | | | TERM + GM-UBQ PRO-V1::GM-UBQ |
| | | | 5’ UTR::GM-UBQ |
| | | | INTRON1::CTP::SPCN::UBQ14 |
| | | | TERM + GM-EF1A2 PRO:: GM-EF1A2 |
| | | | 5’ UTR:: GM-EF1A2 INTRON1:: GM- |
| | | | EF1A2 5’ UTR::DS-RED2::UBQ3 |
| | | | TERM + LB |
| 171 | DNA | RV027337 | T-DNA with AT-ANL2 PRO |
| | | | Synthetic construct comprising the T- |
| | | | DNA (RB to LB): RB + AT-ANL2 |
| | | | PRO::HA-WUS::OS-T28 TERM + GM- |
| | | | UBQ PRO::GM-UBQ 5’ UTR::GM- |
| | | | UBQ INTRON1::ZS-YELLOW1 |
| | | | N1::NOS TERM + AT-UBIQ10 |
| | | | PRO::AT-UBIQ10 5’ UTR::AT-UBIQ10 |
| | | | INTRON1::CTP::SPCN::UBQ14 |
| | | | TERM + GM-EF1A2 PRO::GM-EF1A2 |
| | | | 5’ UTR::GM-EF1A2 INTRON1::DS- |
| | | | RED2::UBQ TERM + LB |
| 172 | DNA | RV027338 | T-DNA with AT-CER6 PRO |
| | | | Synthetic construct comprising the T- |
| | | | DNA (RB to LB): RB + AT-CER6 |
| | | | PRO::HA-WUS::OS-T28 TERM + GM- |
| | | | UBQ PRO::GM-UBQ 5’ UTR::GM- |
| | | | UBQ INTRON1::ZS-YELLOW1 |
| | | | N1::NOS TERM + AT-UBIQ10 |
| | | | PRO::AT-UBIQ10 5’ UTR::AT-UBIQ10 |
| | | | INTRON1::CTP::SPCN::UBQ14 |
| | | | TERM + GM-EF1A2 PRO::GM-EF1A2 |
| | | | 5’ UTR::GM-EF1A2 INTRON1::DS- |
| | | | RED2::UBQ TERM + LB |
| 173 | DNA | RV027340 | T-DNA with AT-HDG2 PRO |
| | | | Synthetic construct comprising the T- |
| | | | DNA (RB to LB): RB + AT-HDG2 |
| | | | PRO::HA-WUS::OS-T28 TERM + GM- |
| | | | UBQ PRO::GM-UBQ 5’ UTR::GM- |
| | | | UBQ INTRON1::ZS-YELLOW1 |
| | | | N1::NOS TERM + AT-UBIQ10 |
| | | | PRO::AT-UBIQ10 5’ UTR::AT-UBIQ10 |
| | | | INTRON1::CTP::SPCN::UBQ14 |
| | | | TERM + GM-EF1A2 PRO::GM-EF1A2 |
| | | | 5’ UTR::GM-EF1A2INTRON1::DS- |
| | | | RED2::UBQ TERM + LB |
| 174 | DNA | RV027342 | T-DNA with AT-ML1 PRO |
| | | | Synthetic construct comprising the T- |
| | | | DNA (RB to LB): RB + AT-ML1 |
| | | | PRO::HA-WUS::OS-T28 TERM + GM- |
| | | | UBQ PRO::GM-UBQ 5’ UTR::GM- |
| | | | UBQ INTRON1::ZS-YELLOW1 |
| | | | N1::NOS TERM + AT-UBIQ10 |
| | | | PRO::AT-UBIQ10 5’ UTR::AT-UBIQ10 |
| | | | INTRON1::CTP::SPCN::UBQ14 |
| | | | TERM + GM-EF1A2 PRO::GM-EF1A2 |
| | | | 5’ UTR::GM-EF1A2 INTRON1::DS- |
| | | | RED2::UBQ TERM + LB |
| 175 | DNA | RV027343 | T-DNA with AT-PDF1 PRO |
| | | | Synthetic construct comprising the T- |
| | | | DNA (RB to LB): RB + AT-PDF1 |
| | | | PRO::HA-WUS::OS-T28 TERM + GM- |
| | | | UBQ PRO::GM-UBQ 5’ UTR::GM- |
| | | | UBQ INTRON1::ZS-YELLOW1 |
| | | | N1::NOS TERM + AT-UBIQ10 |
| | | | PRO::AT-UBIQ10 5’ UTR::AT-UBIQ10 |
| | | | INTRON1::CTP::SPCN::UBQ14 |
| | | | TERM + GM-EF1A2 PRO::GM-EF1A2 |
| | | | 5’ UTR::GM-EF1A2 INTRON1::DS- |
| | | | RED2::UBQ TERM +LB |
| 176 | DNA | PCR Target1 | Synthetic sequence PCR Target1 |
| 177 | DNA | PCR Target2 | Synthetic sequence PCR Target2 |
| 178 | DNA | PCR Target3 | Synthetic sequence PCR Target3 |
| 179 | DNA | PCR Target4 | Synthetic sequence PCR Target4 |
| 180 | DNA | PCR Target5 | Synthetic sequence PCR Target5 |
| 181 | DNA | PCR Target6 | Synthetic sequence PCR Target6 |
| 182 | DNA | PCR Target7 | Synthetic sequence PCR Target7 |
| 183 | DNA | PCR Target8 | Synthetic sequence PCR Target8 |
| 184 | DNA | PCR Target9 | Synthetic sequence PCR Target9 |
| 185 | DNA | PCR Target10 | Synthetic sequence PCR Target10 |
| 186 | DNA | PCR Target11 | Synthetic sequence PCR Target11 |
| 187 | DNA | PCR Target12 | Synthetic sequence PCR Target12 |
| 188 | DNA | PCR Target13 | Synthetic sequence PCR Target13 |
| 189 | DNA | AT-CER6 | Arabidopsis thaliana CER6 |
| | | (TRUNCATED2) | (ECERIFERUM6) promoter sequence |
| | | | (TRUNCATED2) |
| 190 | DNA | RV029482 | T-DNA with ALA-NLS-GG-WUS + |
| | | | HSP:CRE |
| | | | Synthetic construct comprising the T- |
| | | | DNA (RB to LB): RB + LOXP + AT- |
| | | | UBIQ10 PRO::AT-NLS:GG-WUS::OS- |
| | | | T28 TERM + AT-HSP811L PRO::MO- |
| | | | CRE EXON1:ST-LS1 INTRON2:MO- |
| | | | CRE EXON2::SB-GKAF TERM + |
| | | | LOXP + GM-UBQ PRO-V1:GM-UBQ |
| | | | 5UTR:QM-UBQ |
| | | | INTRON1::CTP:SPCN::UBQ14 |
| | | | TERM + GMEF1A2 PRO::GM-EF1A2 |
| | | | 5UTR1::GM-EF1A2 INTRON1::GM- |
| | | | EF1A2 5UTR2::DS-RED2::UBQ3 |
| | | | TERM + LB |
| 191 | DNA | RV029481 | T-DNA with GG-WUS + HSP:CRE + |
| | | | IPT |
| | | | Synthetic construct comprising the T- |
| | | | DNA (RB to LB): RB + LOXP + AT- |
| | | | UBIQ10 PRO::GG-WUS::OS-T28 |
| | | | TERM + AT-UBI14 PRO:AT-UBI14 |
| | | | 5UTR:AT-UBI14 |
| | | | INTRON::IPT::PHASEOLIN TERM + |
| | | | AT-HSP811L PRO::MO-CRE |
| | | | EXON1:ST-LS1 INTRON2:MO-CRE |
| | | | EXON2::SB-GKAF TERM + LOXP + |
| | | | GM-UBQ PRO-V1:GM-UBQ |
| | | | 5UTR:QM-UBQ |
| | | | INTRON1::CTP:SPCN::UBQ14 |
| | | | TERM + GMEF1A2 PRO::GM-EF1A2 |
| | | | 5UTR1::GM-EF1A2 INTRON1:: GM- |
| | | | EF1A2 5UTR2::DS-RED2::UBQ3 |
| | | | TERM + LB |
| 192 | DNA | RV029631 | T-DNA with GG-WUS + HSP:CRE |
| | | | Synthetic construct comprising the T- |
| | | | DNA (RB to LB): RB + LOXP + AT- |
| | | | UBIQ10 PRO::GG-WUS::OS-T28 |
| | | | TERM + AT-HSP811L PRO::MO-CRE |
| | | | EXON1:ST-LS1 INTRON2:MO-CRE |
| | | | EXON2::SB-GKAF TERM + LOXP + |
| | | | GM-UBQ PRO-V1:GM-UBQ |
| | | | 5UTR:QM-UBQ |
| | | | INTRON1::CTP:SPCN::UBQ14 |
| | | | TERM + GMEF1A2 PRO::GM-EF1A2 |
| | | | 5UTR1::GM-EF1A2 INTRON1:: GM- |
| | | | EF1A2 5UTR2::DS-RED2::UBQ3 |
| | | | TERM + LB |
| 193 | DNA | RV029630 | T-DNA with PT-WUS + HSP:CRE |
| | | | Synthetic construct comprising the T- |
| | | | DNA (RB to LB): RB + LOXP + AT- |
| | | | UBIQ10 PRO::PT-WUS::OS-T28 |
| | | | TERM + AT-HSP811L PRO::MO-CRE |
| | | | EXON1:ST-LS1 INTRON2:MO-CRE |
| | | | EXON2::SB-GKAF TERM + LOXP + |
| | | | GM-UBQ PRO-V1:GM-UBQ |
| | | | 5UTR:QM-UBQ |
| | | | INTRON1::CTP:SPCN::UBQ14 |
| | | | TERM + GMEF1A2 PRO::GM-EF1A2 |
| | | | 5UTR1::GM-EF1A2 INTRON1:: GM- |
| | | | EF1A2 5UTR2::DS-RED2::UBQ3 |
| | | | TERM + LB |
| 194 | DNA | RV029480 | T-DNA with ALA-NLS-PT -WUS + |
| | | | HSP:CRE |
| | | | Synthetic construct comprising the T- |
| | | | DNA (RB to LB): RB + LOXP + AT- |
| | | | UBIQ10 PRO::NLS:PT-WUS::OS-T28 |
| | | | TERM + AT-HSP811L PRO::MO-CRE |
| | | | EXON1:ST-LS1 INTRON2:MO-CRE |
| | | | EXON2::SB-GKAF TERM + LOXP + |
| | | | GM-UBQ PRO-V1:GM-UBQ |
| | | | 5UTR:QM-UBQ |
| | | | INTRON1::CTP:SPCN::UBQ14 |
| | | | TERM + GMEF1A2 PRO::GM-EF1A2 |
| | | | 5UTR1::GM-EF1A2 INTRON1:: GM- |
| | | | EF1A2 5UTR2::DS-RED2::UBQ3 |
| | | | TERM + LB |
| 195 | DNA | RV029479 | T-DNA with PT-WUS + HSP:CRE + IPT |
| | | | Synthetic construct comprising the T- |
| | | | DNA (RB to LB): RB+ LOXP + AT- |
| | | | UBIQ10 PRO:: PT-WUS::OS-T28 |
| | | | TERM + AT-UBI14 PRO:AT-UBI14 |
| | | | 5UTR:AT-UBI14 |
| | | | INTRON::IPT::PHASEOLIN TERM + |
| | | | AT-HSP811L PRO::MO-CRE |
| | | | EXON1:ST-LS1 INTRON2:MO-CRE |
| | | | EXON2::SB-GKAF TERM + LOXP + |
| | | | GM-UBQ PRO-V1:GM-UBQ |
| | | | 5UTR:QM-UBQ |
| | | | INTRON1::CTP:SPCN::UBQ14 |
| | | | TERM + GMEF1A2 PRO::GM-EF1A2 |
| | | | 5UTR1::GM-EF1A2 INTRON1:: GM- |
| | | | EF1A2 5UTR2::DS-RED2::UBQ3 |
| | | | TERM + LB |
| 196 | DNA | RV026592 | Synthetic construct comprising the T- |
| | | | DNA with Solanum lycopersicum WUS |
| | | | gene (SL-WUS) (RB to LB): |
| | | | RB + CAMV35S PRO::SL-WUS- |
| | | | V1::OS-T28 TERM + GM-UBQ |
| | | | PRO::GM-UBQ 5’ UTR::GM-UBQ |
| | | | INTRON1::ZS-YELLOW1 N1::NOS |
| | | | TERM + GM-UBQ PRO::GM-UBQ 5’ |
| | | | UTR::GM-UBQ |
| | | | INTRON1::CTP::SPCN::UBQ14 |
| | | | TERM + GM-EF1A2 PRO::GM-EF1A2 |
| | | | 5’ UTR::GM-EF1A2 INTRON1::DS- |
| | | | RED2::UBQ TERM + LB |
| 197 | DNA | SL-WUS | WUS ortholog of Solanum lycopersicum |
| | | | with KpnI site replaced by changing C to |
| | | | T at 762bpcoding sequence |
| 198 | PRT | SL-WUS | Solanum lycopersicum WUS protein |
| | | | sequence |

The following examples are offered by way of illustration and not by way of limitation.

### EXAMPLES

The aspects of the disclosure are further defined in the following Examples, in which parts and percentages are by weight and degrees are Celsius, unless otherwise stated. These Examples, while indicating aspects of the disclosure, are given by way of illustration only. From the above discussion and these Examples, one skilled in the art can ascertain the essential characteristics of the aspects of the disclosure, and without departing from the spirit and scope thereof, can make various changes and modifications of them to adapt to various usages and conditions. Thus, various modifications in addition to those shown and described herein will be apparent to those skilled in the art from the foregoing description. Such modifications are also intended to fall within the scope of the appended claims.

### Example 1: Plant Materials and Media Compositions

A wide range of tissue or explant types can be used in the current method, including vegetative plant organs and their composite tissues including but are not limited to leaf explants, leaf primordia, stipule, cotyledons, cotyledonary nodes, mesocotyl, stem explants, primary roots, lateral secondary roots, root segments, buds, and meristems, including but not limited to apical meristems, root meristems, secondary meristems, axillary meristems, and floral meristems. The compositions of various media used in soybean transformation, tissue culture and regeneration are outlined in Table 4. In this table, medium M1 is used for initiation of suspension cultures, if this is the starting material for transformation. Media M2 and M3 represent typical co-cultivation media useful for Agrobacterium transformation of the entire range of explants listed above. Medium M4 is useful for selection (with the appropriate selective agent), M5 is used for somatic embryo maturation, and medium M6 is used for germination to produce T0 plantlets.

TABLE 4
Composition of Cultivation Media M1-M6.

| | | | | | |
|---|---|---|---|---|---|
| M1 | M2 | M3 | M4 | M5 | M6 |

| | | | | | | | | | | | | |
|---|---|---|---|---|---|---|---|---|---|---|---|---|
| MS salt with | 4.44 | g/L | | | | | 4.4 | g/L | 4.44 | g/L | | |
| B5 vitamins | | | | | | | | | | | | |
| (PhytoTech | | | | | | | | | | | | |
| M404) | | | | | | | | | | | | |
| Gamborg B-5 | | | | | | | | | | | 3.21 | g/L |
| basal medium | | | | | | | | | | | | |
| (PhytoTech | | | | | | | | | | | | |
| G398) | | | | | | | | | | | | |
| Modified MS | | | 2.68 | g/L | 2.68 | g/L | | | | | | |
| salt | | | | | | | | | | | | |
| (PhytoTech | | | | | | | | | | | | |
| M571) | | | | | | | | | | | | |
| B5 vitamins | | | 1 | ml | 1 | ml | 1 | ml | | | | |
| (1000X) | | | | | | | | | | | | |
| (PhytoTech | | | | | | | | | | | | |
| G249) | | | | | | | | | | | | |
| 2,4-D stock 10 | 4 | ml | 1 | ml | 1 | ml | 4 | ml | | | | |
| mg/ml | | | | | | | | | | | | |
| KNO₃ | | | 1.64 | g/L | 1.64 | g/L | | | | | | |
| (NH₄)₂SO₄ | | | 0.463 | g/L | 0.463 | g/L | | | | | | |
| Asparagine | | | 1 | g/L | 1 | g/L | | | | | | |
| Sucrose | | | 68.5 | g/L | 85.6 | g/L | | | | | 20 | g/L |
| Glucose | 31.5 | g/L | 36 | g/L | 49.6 | g/L | 31.5 | g/l | | | | |
| Maltose | | | | | | | | | 60 | g/L | | |
| MgCl₂•6H₂O | | | | | | | | | 0.75 | g/L | | |
| Activated | | | | | | | | | 5 | g/L | | |
| charcoal | | | | | | | | | | | | |
| (PhytoTech | | | | | | | | | | | | |
| C325) | | | | | | | | | | | | |
| Casein | | | 1 | g/L | 1 | g/L | | | | | | |
| hydrolysate | | | | | | | | | | | | |
| (PhytoTech | | | | | | | | | | | | |
| C184) | | | | | | | | | | | | |

| | | | | | | |
|---|---|---|---|---|---|---|
| pH | 7.0 | 5.4 | 5.4 | 7.0 | 5.7 | 5.7 |

| | | | | | | | | | | | | |
|---|---|---|---|---|---|---|---|---|---|---|---|---|
| Acetosyringone | | | 300 | μM | 300 | μM | | | | | | |
| TC agar | 4 | g/L | | | | | | | | | 5 | g/L |
| Gelrite (Plant | | | | | | | | | | | | |
| Media Cat# | | | | | | | 2 | g/l | 2 | g/L | | |
| 714246) | | | | | | | | | | | | |

After 1-5 days of co-culture, the tissue is cultured on M3 medium with no selection for one week (recovery period), and then moved onto selection. For selection, an antibiotic or herbicide is added to M3 medium for the selection of stable transformants. To begin counter-selection against Agrobacterium, 300 mg/l Timentin® (sterile ticarcillin disodium mixed with clavulanate potassium, PlantMedia, Dublin, Ohio, USA) is also added, and both the selective agent and Timentin® are maintained in the medium throughout selection (up to total 8 weeks). The selective media is replaced weekly. After 6-8 weeks on selective medium, transformed tissue becomes visible as green tissue against a background of bleached (or necrotic), less healthy tissue. These pieces of tissue are cultured for an additional 4-8 weeks.

Green and healthy somatic embryos are then transferred to M5 media containing 100 mg/L Timentin®. After a total of 4 weeks of maturation on M5 media, mature somatic embryos are placed in a sterile, empty Petri dish, sealed with Micropore™ tape (3M Health Care, St. Paul, Minn., USA) or placed in a plastic box (with no fiber tape) for 4-7 days at room temperature.

Desiccated embryos are planted in M6 media where they are left to germinate at 26° C. with an 18-hour photoperiod at 60-100 μE/m²/s light intensity. After 4-6 weeks in germination media, the plantlets are transferred to moistened Jiffy-7 peat pellets (Jiffy Products Ltd, Shippagan, Canada), and kept enclosed in clear plastic tray boxes until acclimatized in a Percival incubator under the following conditions, a 16-hour photoperiod at 60-100 μE/m²/s, 26° C./24° C. day/night temperatures. Finally, hardened plantlets are potted in 2-gallon pots containing moistened SunGro 702 and grown to maturity, bearing seed, in a greenhouse.

### Example 2: Transformation of Soybean

Standard protocols for particle bombardment (Finer and McMullen, 1991, In Vitro Cell Dev. Biol.-Plant 27:175-182), Agrobacterium-mediated transformation (Jia et al., 2015, Int J. Mol. Sci. 16:18552-18543; US20170121722 incorporated herein by reference in its entirety), Ochrobactrum-mediated transformation (US20180216123 incorporated herein by reference in its entirety) or Rhizobiaceae-mediated transformation (U.S. Pat. No. 9,365,859 incorporated herein by reference in its entirety) for soybean can be used with the methods of the disclosure.

Media useful in soybean transformation are listed in Table 5.

TABLE 5

Infection medium (101S): 1 g/L L-Asparagine; 0.4630 g/L
Ammonium Sulfate; 1.64 g/L Potassium Nitrate; 68.5 g/L
Sucrose; 36 g/L Glucose; 2.68 g/L Modified Basal Salt Mixture;
1 g/L Casein Hydrolysate (Acid); 1 ml/L 36N-Gamborg B5
Vitamins 1000X; 10 mg/ml 2,4-D; pH 5.4.
Co-cultivation medium (562V): 4 g/L CHU(N6) basal salts;
1 ml/L1000x Erikssons vitamins; 1.25 ml/L 49B-Thiamine
(0.4 mg/ml); 30 g/L Sucrose; 4 ml/L 2,4-D (0.5 mg/ml);
0.69 g/L L-Proline; 8g/L Sigma Agar; pH 5.8.
Shoot induction medium no selection (630E): 4.05 g/L Rugini
Olive Medium Mix; 30 g/L Sucrose; 6 g/L TC Agar; 1.11 ml/L
BAP (1 mg/ml); 1 ml/L Cefotaxime (250 mg/ml); pH 5.6.
Shoot Induction Auxin media (199A): 4.44 g/L MS modified
basal medium with gamborg vitamins; 4 ml/L 2,4-D (10 mg/ml);
30 g/L Sucrose; 2 g/L Gelzan; pH 7.
Rooting medium (640G): 2.22 g/L MS modified basal medium
with Gamborg vitamins; 20 g/L Sucrose; 0.64 g/L MES buffer;
5 ml/L NaFe EDTA for B5H 100X; 6 g/L TC Agar; pH
5.6; 0.05 ml/L Cefotaxime (250 mg/ml).

### Example 3: The GM-Hbstart3 or the Gm-Ltp3 Promoter

Controlling Expression of Morphogenic Genes Improves Transformation

Using the GM-HBSTART3 promoter (SEQ ID NO: 1), the GM-LTP3 promoter (SEQ ID NO:124) or any of the other promoters disclosed herein to drive expression of an Arabidopsis LEC1, LEC2, KN1, STM or LEC1-like (Kwong et al., (2003) The Plant Cell, Vol. 15, 5-18) gene in expression cassettes comprising a fluorescent marker, is found to increase the frequency of somatic embryo formation and the recovery of transgenic T0 plants. Agrobacterium strain LBA4404 is used to transform various vegetative plant organs and their composite tissues including but are not limited to leaf explants, leaf primordia, stipule, cotyledons, cotyledonary nodes, mesocotyl, stem explants, primary roots, lateral secondary roots, root segments, buds, and meristems, including but not limited to apical meristems, root meristems, secondary meristems, axillary meristems, and floral meristems of Pioneer soybean variety PHY21. Four days after the Agrobacterium infection is started, the tissue is washed with sterile culture medium to remove excess bacteria. It is expected that approximately nine days later the tissue is moved to somatic embryo maturation medium, and approximately twenty-two days after that the transgenic somatic embryos are ready for dry-down. At this point, well-formed, mature somatic embryos fluoresce under an epifluorescence stereo-microscope with an appropriate filter set. The somatic embryos that develop are functional and germinate to produce healthy plants in the greenhouse. It is expected that this rapid method of producing somatic embryos and germinating to form plants reduces the typical timeframe from Agrobacterium infection to moving transgenic T0 plants into the greenhouse from four months (for conventional soybean transformation) to approximately two to three months.

### Example 4: Use of the Gm-Hbstart3 or the Gm-Ltp3 Promoter to Control Expression of the Agrobacterium Ipt Gene Stimulates Direct Shoot Formation and Improves Transformation

Using the GM-HBSTART3 promoter (SEQ ID NO: 1), the GM-LTP3 promoter (SEQ ID NO:124) or any of the other promoters disclosed herein to drive expression of an Agrobacterium IPT gene in expression cassettes comprising a fluorescent marker, is found to increase the frequency of multiple shoot formation and the recovery of transgenic T0 plants. Agrobacterium strain LBA4404 is used to transform various vegetative plant organs and their composite tissues including but are not limited to leaf explants, leaf primordia, stipule, cotyledons, cotyledonary nodes, mesocotyl, stem explants, primary roots, lateral secondary roots, root segments, buds, and meristems, including but not limited to apical meristems, root meristems, secondary meristems, axillary meristems, and floral meristems of Pioneer soybean variety PHY21. Four days after the Agrobacterium infection is started, the tissue is washed with sterile culture medium to remove excess bacteria and moved onto medium that promotes multiple shoot proliferation. It is expected that nine days later the tissue is moved to medium that favors shoot development, and twenty-two after that the transgenic shoots are moved onto medium that promotes rooting. At this point, incipient plantlets fluoresce under an epifluorescence stereo-microscope with an appropriate filter set. Functional plantlets develop rapidly and continue to grow and produce healthy plants in the greenhouse. It is expected that this rapid method of directly forming transgenic plants reduces the typical timeframe from Agrobacterium infection to moving transgenic T0 plants into the greenhouse from four months (for conventional soybean transformation) to approximately two to three months.

### Example 5: The GM-HBSTART3 or the GM-LTP3 Promoter Controlling Expression of the Arabidopsis Monopteros-Delta Gene Stimulates Direct Shoot Formation and Improves Transformation

Using the GM-HBSTART3 promoter (SEQ ID NO: 1), the GM-LTP3 promoter (SEQ ID NO:124) or any of the other promoters disclosed herein to drive expression of an Arabidopsis MONOPTEROS-DELTA gene in expression cassettes comprising a fluorescent marker, is found to increase the frequency of multiple shoot formation and the recovery of transgenic T0 plants. Agrobacterium strain LBA4404 is used to transform various vegetative plant organs and their composite tissues including but are not limited to leaf explants, leaf primordia, stipule, cotyledons, cotyledonary nodes, mesocotyl, stem explants, primary roots, lateral secondary roots, root segments, buds, and meristems, including but not limited to apical meristems, root meristems, secondary meristems, axillary meristems, and floral meristems of Pioneer soybean variety PHY21. Four days after the Agrobacterium infection is started, the tissue is washed with sterile culture medium to remove excess bacteria and moved onto medium that promotes multiple shoot proliferation. It is expected that nine days later the tissue is moved to medium that favors shoot development, and twenty-two days after that the transgenic shoots are moved onto medium that promotes rooting. At this point, incipient plantlets fluoresce under an epifluorescence stereo-microscope with an appropriate filter set. Functional plantlets develop rapidly and continue to grow and produce healthy plants in the greenhouse. It is expected that this rapid method of directly forming transgenic plants reduces the typical timeframe from Agrobacterium infection to moving transgenic T0 plants into the greenhouse from four months (for conventional soybean transformation) to approximately two to three months.

### Example 6: The GM-HBSTART3 or the GM-LTP3 Promoter Controlling Expression of Growth Enhancing Genes Improves Transformation

Using the GM-HBSTART3 promoter (SEQ ID NO: 1), the GM-LTP3 promoter (SEQ ID NO:124) or any of the other promoters disclosed herein to drive expression of an Agrobacterium AV-6B gene, an Agrobacterium IAA-h gene, an Agrobacterium IAA-m gene, an Arabidopsis SERK or an Arabidopsis AGL15 gene in expression cassettes comprising a fluorescent marker, is found to increase the frequency of somatic embryo formation and the recovery of transgenic T0 plants. Agrobacterium strain LBA4404 is used to transform various vegetative plant organs and their composite tissues including but are not limited to leaf explants, leaf primordia, stipule, cotyledons, cotyledonary nodes, mesocotyl, stem explants, primary roots, lateral secondary roots, root segments, buds, and meristems, including but not limited to apical meristems, root meristems, secondary meristems, axillary meristems, and floral meristems of Pioneer soybean variety PHY21. Four days after the Agrobacterium infection is started, the tissue is washed with sterile culture medium to remove excess bacteria. It is expected that nine days later the tissue is moved to somatic embryo maturation medium, and twenty-two days after that the transgenic somatic embryos are ready for dry-down. At this point, well-formed, mature somatic embryos fluoresce under an epifluorescence stereo-microscope with an appropriate filter set. The somatic embryos that develop are functional and germinate to produce healthy plants in the greenhouse. It is expected that this rapid method of producing somatic embryos and germinating to form plants reduces the typical timeframe from Agrobacterium infection to moving transgenic T0 plants into the greenhouse from four months (for conventional soybean transformation) to approximately two to three months.

### Example 7: Use of Viral Enhancer Elements in Conjunction with the GM-HBSTART3 Promoter to Drive Expression of Wus Results in Further Improvement of Soy Transformation

Relative to using the GM-HBSTART3 promoter (SEQ ID NO: 1), the GM-LTP3 promoter (SEQ ID NO:124) or any of the other promoters disclosed herein alone, use of a viral enhancer element such as the 35S enhancer adjacent to the GM-HBSTART3 promoter (SEQ ID NO: 1), the GM-LTP3 promoter (SEQ ID NO:124) or any of the other promoters disclosed herein to drive expression of WUS in expression cassettes comprising a fluorescent marker, results in a further increase in the frequency of somatic embryo formation and the frequency of somatic embryo maturation, resulting in an overall increase in the recovery of transgenic T0 plants. Agrobacterium strain LBA4404 is used to transform various vegetative plant organs and their composite tissues including but are not limited to leaf explants, leaf primordia, stipule, cotyledons, cotyledonary nodes, mesocotyl, stem explants, primary roots, lateral secondary roots, root segments, buds, and meristems, including but not limited to apical meristems, root meristems, secondary meristems, axillary meristems, and floral meristems of Pioneer soybean variety PHY21. Four days after the Agrobacterium infection is started, the tissue is washed with sterile culture medium to remove excess bacteria. Nine days later the tissue is moved to somatic embryo maturation medium, and twenty-two days after that transgenic somatic embryos are ready for dry-down. At this point, well-formed, mature somatic embryos fluoresce under an epifluorescence stereo-microscope with an appropriate filter set. The somatic embryos that develop are functional and germinate to produce healthy plants in the greenhouse. This rapid method of producing somatic embryos and germinating to form plants reduces the typical timeframe from Agrobacterium infection to moving transgenic T0 plants into the greenhouse from four months (for conventional soybean transformation) to approximately two to three months.

Other enhancer elements are tested in a similar fashion and are shown to also result in increased transformation relative to using the GM-HBSTART3 promoter (SEQ ID NO: 1), the GM-LTP3 promoter (SEQ ID NO:124) or any of the other promoters disclosed herein alone. These enhancers include the viral enhancers such as the Cauliflower Mosaic Virus 35S and the Mirabilis Mosaic Virus 2×MMV as well as endogenous plant enhancer elements.

### Example 8: Orthologs of the Arabidopsis Wus Gene Function in Brassica to Stimulate Somatic Embryo Formation

The following particle gun transformation treatments are compared. All particle gun transformation treatments contain plasmid QC318 (SEQ ID NO: 117) with GM-EF1 A PRO::GM-EF1 A INTRON1::ZS-YELLOW::NOS TERM+GM-SAMS PRO::GM-SAMS INTRON1::GM-ALS::GM-ALS TERM. The treatments include 1) a control with no addition genes, 2) pVER9662 (SEQ ID NO: 118) with the AT-UBI PRO driving expression of the Arabidopsis WUS gene, 3) UBIGMWUS (SEQ ID NO: 119) with the AT-UBI PRO driving expression of the Glycine max WUS gene, 4) UBIMTWUS (SEQ ID NO: 120) with the AT-UBI PRO driving expression of the Medicago truncatula WUS gene, 5) UBILJWUS (SEQ ID NO: 121) with the AT-UBI PRO driving expression of the Lotus japonica WUS gene, 6) UBIPVWUS (SEQ ID NO: 122) with the AT-UBI PRO driving expression of the Phaseolus vulgaris WUS gene, and 7) UBIPHWUS (SEQ ID NO: 123) with the AT-UBI PRO driving expression of the petunia WUS gene.

Various vegetative plant organs and their composite tissues including but are not limited to leaf explants, leaf primordia, stipule, cotyledons, cotyledonary nodes, mesocotyl, stem explants, primary roots, lateral secondary roots, root segments, buds, and meristems, including but not limited to apical meristems, root meristems, secondary meristems, axillary meristems, and floral meristems of Brassica are isolated for transformation with the particle gun. A mixture of two plasmids are co-introduced, the first containing an expression cassette consisting of the AT-UBI PRO driving expression of the cDNA sequence for each of the WUS orthologs (pVER9662 (SEQ ID NO: 118), UBIGMWUS (SEQ ID NO: 119), UBIMTWUS (SEQ ID NO: 120), UBILJWUS (SEQ ID NO: 121), UBIPVWUS (SEQ ID NO: 122), and UBIPHWUS (SEQ ID NO: 123) plus an expression cassette for ZS-YELLOW (QC318 (SEQ ID NO: 117)). The explants are cultured for two weeks. At two weeks, the number of fluorescing globular somatic embryos are counted and tabulated for each treatment.

Two weeks after particle gun transformation, fluorescent globular somatic embryos are rarely observed on the bombarded control vegetative plant organs and their composite tissues, including immature cotyledons, split seeds, isolated embryonic axes, mature cotyledonary nodes, hypocotyl, epicotyl, or leaf tissue of Brassica, while for all the other treatments (containing WUS genes from different dicot species driven by the AT-UBI promoter), numerous fluorescent somatic embryos are observed on bombarded vegetative plant organs and their composite tissues including but are not limited to leaf explants, leaf primordia, stipule, cotyledons, cotyledonary nodes, mesocotyl, stem explants, primary roots, lateral secondary roots, root segments, buds, and meristems, including but not limited to apical meristems, root meristems, secondary meristems, axillary meristems, and floral meristems of Brassica.

### Example 9: Orthologs of the Arabidopsis Wus Gene Function in Sunflower to Stimulate Somatic Embryo Formation

The following particle gun transformation treatments are compared. All particle gun transformation treatments contain plasmid QC318 (SEQ ID NO: 117) with GM-EF1 A PRO::GM-EF1 A INTRON1::ZS-YELLOW::NOS TERM+GM-SAMS PRO::GM-SAMS INTRON1::GM-ALS::GM-ALS TERM. The treatments include 1) a control with no addition genes, 2) pVER9662 (SEQ ID NO: 118) with the AT-UBI PRO driving expression of the Arabidopsis WUS gene, 3) UBIGMWUS (SEQ ID NO: 119) with the AT-UBI PRO driving expression of the Glycine max WUS gene, 4) UBIMTWUS (SEQ ID NO: 120) with the AT-UBI PRO driving expression of the Medicago truncatula WUS gene, 5) UBILJWUS (SEQ ID NO: 121) with the AT-UBI PRO driving expression of the Lotus japonica WUS gene, 6) UBIPVWUS (SEQ ID NO: 122) with the AT-UBI PRO driving expression of the Phaseolus vulgaris WUS gene, and 7) UBIPHWUS (SEQ ID NO: 123) with the AT-UBI PRO driving expression of the petunia WUS gene.

Various vegetative plant organs and their composite tissues including but are not limited to leaf explants, leaf primordia, stipule, cotyledons, cotyledonary nodes, mesocotyl, stem explants, primary roots, lateral secondary roots, root segments, buds, and meristems, including but not limited to apical meristems, root meristems, secondary meristems, axillary meristems, and floral meristems of sunflower are isolated for transformation with the particle gun. A mixture of two plasmids are co-introduced, the first containing an expression cassette consisting of the AT-UBI PRO driving expression of the cDNA sequence for each of the WUS orthologs (pVER9662 (SEQ ID NO: 118), UBIGMWUS (SEQ ID NO: 119), UBIMTWUS (SEQ ID NO: 120), UBILJWUS (SEQ ID NO: 121), UBIPVWUS (SEQ ID NO: 122), and UBIPHWUS (SEQ ID NO: 123)) plus an expression cassette for ZS-YELLOW (QC318 (SEQ ID NO: 117)). The explants are cultured for two weeks. At two weeks, the number of fluorescing globular somatic embryos are counted and tabulated for each treatment.

Two weeks after particle gun transformation, fluorescent globular somatic embryos are rarely observed on the bombarded control vegetative plant organs and their composite tissues of sunflower, while for all the other treatments (containing WUS genes from different dicot species driven by the AT-UBI promoter), numerous fluorescent somatic embryos are observed on bombarded vegetative plant organs and their composite tissues including but are not limited to leaf explants, leaf primordia, stipule, cotyledons, cotyledonary nodes, mesocotyl, stem explants, primary roots, lateral secondary roots, root segments, buds, and meristems, including but not limited to apical meristems, root meristems, secondary meristems, axillary meristems, and floral meristems of sunflower.

### Example 10: Use of Wus Orthologs from Four Different Species to Stimulate Direct Shoot Formation after Agrobacterium-Mediated Transformation of Soybean Leaf Segments

The Agrobacterium strain LBA4404 THY-containing the poplar WUS (RV026520 (SEQ ID NO: 164) (POPTR-WUS)), the Amaranthus WUS (RV026533 (SEQ ID NO: 165) (AMAHY-WUS)), the WUS gene from apple (RV026531 (SEQ ID NO: 157) (MALDO-WUS)) or the WUS gene from Gnetum (RV026522 (SEQ ID NO: 154) (GNEGN-WUS)) was used to transform segments of tissue cut from in vitro-grown, sterile, immature leaves of Pioneer soybean variety PHY21. Agrobacterium strain LBA4404 THY-containing the vectors for the genes listed above and listed in Table 3 were used for the infections, and all bacterial cultures were adjusted to OD 0.5 for infection. All vectors contained the selectable marker gene SPCN (spectinomycin). Leaf explants were infected for 30 min and were placed on co-cultivation medium for three days at 21° C. in dark. After co-cultivation, explants were transferred to shoot regeneration medium for 3 to 4 weeks. Elongated shoots were transferred to rooting media. For the control treatment (transforming with RV022814 (SEQ ID NO: 168) (NO WUS)) which contained the SPCN and ZS-YELLOW1 N1 expression cassettes, no direct shoot formation from leaf segments was observed. However, when transformations were performed which introduced T-DNAs containing the three expression cassettes (WUS, SPCN and ZS-YELLOW1 N1) green healthy shoots were produced from the poplar WUS (RV026520 (SEQ ID NO: 164) (POPTR-WUS)), the Amaranthus WUS (RV026533 (SEQ ID NO: 165) (AMAHY-WUS)), the Gnetum WUS (RV026522 (SEQ ID NO: 154) (GNEGN-WUS)), and the apple WUS (RV026531 (SEQ ID NO: 157) (MALDO-WUS)) (at frequencies of 23.6%, 38.4%, 52% and 52.5%, respectively).

### Example 11: Regeneration from Soy Leaf and Stem Explants Infected with Vectors Containing a Developmental Gene and Spcn Selectable Marker Gene

Soybean lines such as elite lines can be used in this method. Leaf and stem explants of 93Y21 were harvested. Leaves were cut into pieces of uniform size, approximately 30-60 millimeters. Stem internodes were cut into sections of about 0.3 to 0.8 cm in length. Agrobacterium strain LBA4404 THY-containing the vectors listed in Table 6 were used for the infections, and all bacterial cultures were adjusted to OD 0.5 for infection. All vectors contained the selectable marker gene SPCN (spectinomycin). The leaf and stem internode explants were infected for 30 min and were placed on co-cultivation medium for three days at 21° C. in dark. After co-cultivation, explants were transferred to shoot regeneration medium. Infection frequency was evaluated by screening the transient expression of the selectable marker gene at 5 and 20 days after transformation. Shoot regeneration was observed about 30 days after infection (Table 6). Transgenic shoots were evaluated for the presence of the SPCN marker gene. As shown in Table 6 expressing WUS genes from phylogenetically divergent dicot or gymnosperm species stimulated direct shoot formation from either soy leaf or stem explants. Green healthy shoots were produced from either soy leaf or stem explants when transformed with T-DNAs containing the Amaranthus WUS (RV026533 (SEQ ID NO: 165) (AMAHY-WUS)), the poplar WUS (RV026520 (SEQ ID NO: 164) (POPTR-WUS)), the apple WUS (RV026531 (SEQ ID NO: 157) (MALDO-WUS)), and the Gnetum WUS (RV026522 (SEQ ID NO: 154) (GNEGN-WUS)) (at frequencies of 20%, 15%, 5% and 5.5%, respectively).

As shown in Table 6 no shoot induction was observed when Shoot Induction 199 A media was used. These results suggest that improvements in the shoot induction media formulation may further improve the recovery of green healthy shoots.

TABLE 6
Shoot regeneration from infected leaf and stem explants of elite soybean

| | | # Explant | | Marker gene expression | | | | | | | |
|---|---|---|---|---|---|---|---|---|---|---|---|
| | Treatment | used | | at 1 month | | | | Shoots induced | | | |
| Construct | Media | Stems | Leaves | Stems | % | Leaves | % | From leaf | % | From stem | % |
| RV026523 | 630E | 16 | 0 | 4 | 25 | 0 | 0 | 0 | 0 | 0 | 0 |
| (SEQ ID | | | | | | | | | | | |
| NO: 159) | | | | | | | | | | | |
| (KALFE- | | | | | | | | | | | |
| WUS) | | | | | | | | | | | |
| RV026524 | 630E | 10 | 10 | 7 | 70 | 9 | 90 | 0 | 0 | 0 | 0 |
| (SEQ ID | | | | | | | | | | | |
| NO: 158) | | | | | | | | | | | |
| (MANES- | | | | | | | | | | | |
| WUS) | | | | | | | | | | | |
| RV026533 | 630E | 5 | 8 | 1 | 20 | 4 | 50 | 0 | 0 | 1 | 20 |
| (SEQ ID | | | | | | | | | | | |
| NO: 165) | | | | | | | | | | | |
| (AMAHY- | | | | | | | | | | | |
| WUS) | | | | | | | | | | | |
| RV026530 | 630E | 10 | 10 | 1 | 10 | 5 | 50 | 0 | 0 | 0 | 0 |
| (SEQ ID | | | | | | | | | | | |
| NO: 160) | | | | | | | | | | | |
| (GOSHI- | | | | | | | | | | | |
| WUS) | | | | | | | | | | | |
| RV026527 | | | | | | | | | | | |
| (SEQ ID | 630E | 10 | 0 | 0 | 0 | 0 | 0 | 0 | 0 | 0 | 0 |
| NO: 161) | | | | | | | | | | | |
| (ZOSMA- | | | | | | | | | | | |
| WUS) | | | | | | | | | | | |
| RV021090 | 630E | 13 | 0 | 1 | 8 | 0 | 0 | 0 | 0 | 0 | 0 |
| (SEQ ID | | | | | | | | | | | |
| NO: 153) | | | | | | | | | | | |
| (HA-WUS) | | | | | | | | | | | |
| (+Control) | | | | | | | | | | | |
| RV026520 | 630E | 18 | 20 | 1 | 6 | 8 | 40 | 3 | 15 | 0 | 0 |
| (SEQ ID | | | | | | | | | | | |
| NO: 164) | | | | | | | | | | | |
| (POPTR- | | | | | | | | | | | |
| WUS) | | | | | | | | | | | |
| RV026531 | 630E | 20 | 20 | 7 | 35 | 14 | 70 | 1 | 5 | 0 | 0 |
| (SEQ ID | | | | | | | | | | | |
| NO: 157) | | | | | | | | | | | |
| (MALDO- | | | | | | | | | | | |
| WUS) | | | | | | | | | | | |
| RV026534 | 630E | 15 | 15 | 4 | 27 | 1 | 7 | 0 | 0 | 0 | 0 |
| (SEQ ID | | | | | | | | | | | |
| NO: 156) | | | | | | | | | | | |
| (PETHY- | | | | | | | | | | | |
| WUS) | | | | | | | | | | | |
| RV026525 | 630E | 20 | 20 | 1 | 5 | 8 | 40 | 0 | 0 | 0 | 0 |
| (SEQ ID | | | | | | | | | | | |
| NO: 167) | | | | | | | | | | | |
| (PINTA- | | | | | | | | | | | |
| WUS) | | | | | | | | | | | |
| RV022814 | 630E | 20 | 20 | 6 | 30 | 9 | 45 | 0 | 0 | 0 | 0 |
| (SEQ ID | | | | | | | | | | | |
| NO: 168) | | | | | | | | | | | |
| (NO WUS) | | | | | | | | | | | |
| (−Control) | | | | | | | | | | | |
| RV026529 | | | | | | | | | | | |
| (SEQ ID | 630E | 20 | 20 | 1 | 5 | 0 | 0 | 0 | 0 | 0 | 0 |
| NO: 162) | | | | | | | | | | | |
| (AMBTR- | | | | | | | | | | | |
| WUS) | | | | | | | | | | | |
| RV026522 | 630E | 18 | 19 | 6 | 33 | 15 | 79 | 0 | 0 | 1 | 5.5 |
| (SEQ ID | | | | | | | | | | | |
| NO: 154) | | | | | | | | | | | |
| (GNEGN- | | | | | | | | | | | |
| WUS) | | | | | | | | | | | |
| RV026528 | 630E | 18 | 20 | 12 | 67 | 9 | 45 | 0 | 0 | 0 | 0 |
| (SEQ ID | | | | | | | | | | | |
| NO: 163) | | | | | | | | | | | |
| (AQUCO- | | | | | | | | | | | |
| WUS) | | | | | | | | | | | |
| RV026523 | 199A | 16 | 0 | 0 | 0 | 0 | 0 | 0 | 0 | 0 | 0 |
| (SEQ ID | | | | | | | | | | | |
| NO: 159) | | | | | | | | | | | |
| (KALFE- | | | | | | | | | | | |
| WUS) | | | | | | | | | | | |
| RV026524 | 199A | 10 | 10 | 0 | 0 | 4 | 40 | 0 | 0 | 0 | 0 |
| (SEQ ID | | | | | | | | | | | |
| NO: 158) | | | | | | | | | | | |
| (MANES- | | | | | | | | | | | |
| WUS) | | | | | | | | | | | |
| RV026533 | 199A | 5 | 8 | 0 | 0 | 0 | 0 | 0 | 0 | 0 | 0 |
| (SEQ ID | | | | | | | | | | | |
| NO: 165) | | | | | | | | | | | |
| (AMAHY- | | | | | | | | | | | |
| WUS) | | | | | | | | | | | |
| RV026530 | | | | | | | | | | | |
| (SEQ ID | 199A | 10 | 10 | 0 | 0 | 2 | 20 | 0 | 0 | 0 | 0 |
| NO: 160) | | | | | | | | | | | |
| (GOSHI- | | | | | | | | | | | |
| WUS) | | | | | | | | | | | |
| RV026527 | 199A | 10 | 0 | 0 | 0 | 0 | 0 | 0 | 0 | 0 | 0 |
| (SEQ ID | | | | | | | | | | | |
| NO: 161) | | | | | | | | | | | |
| (ZOSMA- | | | | | | | | | | | |
| WUS) | | | | | | | | | | | |
| RV021090 | 199A | 13 | 0 | 0 | 0 | 0 | 0 | 0 | 0 | 0 | 0 |
| (SEQ ID | | | | | | | | | | | |
| NO: 153) | | | | | | | | | | | |
| (HA-WUS) | | | | | | | | | | | |
| (+Control) | | | | | | | | | | | |
| RV026520 | 199A | 18 | 20 | 0 | 0 | 0 | 0 | 0 | 0 | 0 | 0 |
| (SEQ ID | | | | | | | | | | | |
| NO: 164) | | | | | | | | | | | |
| (POPTR- | | | | | | | | | | | |
| WUS) | | | | | | | | | | | |
| RV026531 | 199A | 20 | 20 | 0 | 0 | 2 | 10 | 0 | 0 | 0 | 0 |
| (SEQ ID | | | | | | | | | | | |
| NO: 157) | | | | | | | | | | | |
| (MALDO- | | | | | | | | | | | |
| WUS) | | | | | | | | | | | |
| RV026534 | 199A | 15 | 15 | 0 | 0 | 1 | 7 | 0 | 0 | 0 | 0 |
| (SEQ ID | | | | | | | | | | | |
| NO: 156) | | | | | | | | | | | |
| (PETHY- | | | | | | | | | | | |
| WUS) | | | | | | | | | | | |
| RV026525 | 199A | 20 | 20 | 0 | 0 | 0 | 0 | 0 | 0 | 0 | 0 |
| (SEQ ID | | | | | | | | | | | |
| NO: 167) | | | | | | | | | | | |
| (PINTA- | | | | | | | | | | | |
| WUS) | | | | | | | | | | | |
| RV022814 | 199A | 20 | 20 | 0 | 0 | 0 | 0 | 0 | 0 | 0 | 0 |
| (SEQ ID | | | | | | | | | | | |
| NO: 168) | | | | | | | | | | | |
| (NO WUS) | | | | | | | | | | | |
| (−Control) | | | | | | | | | | | |
| RV026529 | 199A | 20 | 20 | 0 | 0 | 0 | 0 | 0 | 0 | 0 | 0 |
| (SEQ ID | | | | | | | | | | | |
| NO: 162) | | | | | | | | | | | |
| (AMBTR- | | | | | | | | | | | |
| WUS) | | | | | | | | | | | |
| RV026522 | 199A | 18 | 19 | 0 | 0 | 3 | 16 | 0 | 0 | 0 | 0 |
| (SEQ ID | | | | | | | | | | | |
| NO: 154) | | | | | | | | | | | |
| (GNEGN- | | | | | | | | | | | |
| WUS) | | | | | | | | | | | |
| RV026528 | | | | | | | | | | | |
| (SEQ ID | 199A | 18 | 20 | 0 | 0 | 0 | 0 | 0 | 0 | 0 | 0 |
| NO: 163) | | | | | | | | | | | |
| (AQUCO- | | | | | | | | | | | |
| WUS) | | | | | | | | | | | |

### Example 12: Orthologs of the Arabidopsis Wus Gene Stimulate Morphogenic Growth in Brassica Leaf

WUS genes from 12 different dicotyledonous species, 2 gymnosperms, and one monocot species were tested for efficacy by assessing their ability to stimulate growth of transgenic green shoot responses in Brassica while undergoing selection on spectinomycin-containing medium. For all treatments containing a WUS expression cassette, the configuration of the T-DNA was identical with the exception of the WUS gene used in the construct. The T-DNA configuration was RB+CAMV35S PRO::WUS::OS-T28 TERM+GM-UBQ PRO::GM-UBQ SUTR::GM-UBQ INTRON1::ZS-YELLOW1 N1::NOS TERM+AT-UBIQ10 PRO::AT-UBIQ10 SUTR::AT-UBIQ10 INTRON1::CTP::SPCN::UBQ14 TERM+GM-EF1 A2 PRO::GM-EF1 A2 5′ UTR::GM-EF1 A2 INTRON1::DS-RED2::UBQ TERM+LB with the variable WUS gene in bold and italics.

Seeds of Brassica napus were surface sterilized in a 50% Clorox solution and germinated on solid medium containing MS basal salts and vitamins. The seedlings were grown at 28° C. in light, and leaves were collected. The leaves were transferred into 100×25 mm petri plates containing 10 ml of 20 A medium (Table 7) with 200 mM acetosyringone and then sliced into 3-5 mm long sections. After slicing, 40μ1 of Agrobacterium solution (Agrobacterium strain LBA4404 THY-at an OD₅₅₀ of 0.50) containing the expression cassettes described above were added to the plates, and the petri plates containing the leaf/Agrobacterium mixture were either vacuum infiltrated or wounded. The leaf sections placed into a vacuum were exposed to 15 PSI for 1 minute. Wounded leaf sections were pierced 10 times each with a needle. The wounding and vacuum infiltrated plates were moved into dim light and 21° C. for 3 days of co-cultivation.

After co-cultivation, the leaf sections were removed from the Agrobacterium solution, and lightly blotted onto sterile filter paper before placing onto 70 D media (Table 7) and moved to the light room (16 hr. photoperiod at 60-100 μE/m²/s). The leaf sections remained on 70 D media for one week, and then transferred to 70 A media with no spectinomycin. Shoots that emerged were moved onto 70 C shoot elongation media (Table 7) and placed back into the light room (16 hr. photoperiod at 60-100 μE/m²/s) for one month. Seven weeks after initial transformation, shoots were counted.

TABLE 7
Media

Infection and co-cultivation medium (20A): MS salts and vitamins;
0.1 g/L myo-inositol; 0.5 g/L MES buffer; 0.1 mg/L a-NAA;
1 mg/L BAP; 10 ug/L gibberellic acid; 50 mg/L thymidine; pH 5.5.
First culture medium (70D): MS salts and vitamins; 0.1 g/L
myo-inositol; 0.5 g/L MES buffer; 0.1 ml/L a-NAA (1.0 mg/ml);
1 ml/L BAP (1.0 mg/ml); 40 mg/L adenine hemisulfate salt;
20 g/L sucrose; 0.5 g/L PVP40; 1.0 ml/L silver nitrate (2.0 mg/ml);
10 ml/L carbenicillin (50.0 mg/ml); 5 g/L TC agar; pH 5.7.
First Selection medium (70A): MS salts and vitamins; 0.1 g/l
myo-inositol; 0.5 g/l MES buffer; 0.1 mg/l a-NAA; 1 mg/l BAP;
10 ug/l gibberellic acid; 40 mg/l adenine hemisulfate; 20 g/l
sucrose; 0.5 g/l PVP40; 2 mg/l silver nitrate; 0.5 g/l carbenicillin;
5 mg/l spectinomycin; 5 g/l TC agar; pH 5.7.
Second Selection medium (70B): MS salts and vitamins; 0.1 g/l
myo-inositol; 0.5 g/l MES buffer; 0.1 mg/l a-NAA; 1 mg/l BAP;
10 ug/l gibberellic acid; 40 mg/l adenine hemisulfate; 20 g/l
sucrose; 0.5 g/l PVP40; 2 mg/l silver nitrate; 0.5 g/l carbenicillin;
10 mg/l spectinomycin; 5 g/l TC agar; pH 5.7.
Second culture medium (70A no Spec): MS salts and vitamins;
0.1 g/l myo-inositol; 0.5 g/l MES buffer; 0.1 mg/l a-NAA; 1 mg/l
BAP; 10 ug/l gibberellic acid; 40 mg/l adenine hemisulfate; 20
g/l sucrose; 0.5 g/l PVP40; 2 mg/l silver nitrate; 0.5 g/l carbenicillin;
5 g/l TC agar; pH 5.7.
Regeneration Medium (70C): MS salts and vitamins; 0.1 g/l
myo-inositol; 0.5 g/l MES buffer; 2.5 ug/l BAP; 40 mg/l adenine
hemisulfate; 20 g/l sucrose; 0.5 g/l PVP40; 0.5 g/l carbenicillin;
10 mg/l spectinomycin; 5 g/l TC agar; pH 5.7.
Rooting Medium (90A): MS salts and vitamins; 0.5 mg/l IBA;
pH 5.7.

As shown in Table 8 and Table 9, expressing WUS genes from phylogenetically divergent dicot, gymnosperm, or monocot species stimulated direct shoot formation in canola leaf sections. This stimulation of shoot development and the ability to recover spectinomycin-resistant shoots was variable, depending on the source of the WUS gene. For pine (RV026525 (SEQ ID NO: 167) (PINTA-WUS)), cucumber (RV026521 (SEQ ID NO: 166) (CUCSA-WUS)), Amborella (RV026529 (SEQ ID NO: 162) (AMBTR-WUS)), poplar (RV026520 (SEQ ID NO: 164) (POPTR-WUS)), and tomato (RV026592 (SEQ ID NO: 196) (SL-WUS)) this stimulation of transgenic shoots was the same as that seen in the negative control treatment. De novo shoot regeneration ranged for WUS genes from other species up to 29% for cassava (RV026524 (SEQ ID NO: 158) (MANES-WUS)), 25% for both apple (RV026531 (SEQ ID NO: 157) (MALDO-WUS)) and columbine (RV026528 (SEQ ID NO: 163) (AQUCO-WUS)), 21% for both grape (RV026526 (SEQ ID NO: 155) (VITVI-WUS)) and Gnetum gnemon (a gymnosperm) (RV026522 (SEQ ID NO: 154) (GNEGN-WUS)), 17% for petunia (RV026534 (SEQ ID NO: 156) (PETHY-WUS)), 8% for eelgrass (a monocot) (RV026527 (SEQ ID NO: 161) (ZOSMA-WUS)), and 4% for Kalanchoe (RV026523 (SEQ ID NO: 159) (KALFE-WUS)) as shown in Table 8 and Table 9 below.

TABLE 8
Efficacy* of WUS genes from various species in Brassica
leaf tissue

| | Leaf Vacuum Infiltration | | |
|---|---|---|---|
| | | Transgenic | Transgenic |
| Treatment | Leaf Sections | Shoots | Shoots % |
| RV026524 | 14 | 3 | 21% |
| (SEQ ID NO: 158) | | | |
| (MANES-WUS) | | | |
| RV026531 | 12 | 6 | 50% |
| (SEQ ID NO: 157) | | | |
| (MALDO-WUS) | | | |
| RV026528 | 0 | 0 | NA |
| (SEQ ID NO: 163) | | | |
| (AQUCO-WUS) | | | |
| RV026522 | 12 | 6 | 50% |
| (SEQ ID NO: 154) | | | |
| (GNEGN-WUS) | | | |
| RV026526 | 12 | 5 | 42% |
| (SEQ ID NO: 155) | | | |
| (VITVI-WUS) | | | |
| RV026534 | 12 | 4 | 33% |
| (SEQ ID NO: 156) | | | |
| (PETHY-WUS) | | | |
| RV026530 | 12 | 0 | 0% |
| (SEQ ID NO: 160) | | | |
| (GOSHI-WUS) | | | |
| RV026527 | 12 | 0 | 0% |
| (SEQ ID NO: 161) | | | |
| (ZOSMA-WUS) | | | |
| RV026523 | 12 | 1 | 8% |
| (SEQ ID NO: 159) | | | |
| (KALFE-WUS) | | | |
| RV026525 | 12 | 0 | 0% |
| (SEQ ID NO: 167) | | | |
| (PINTA-WUS) | | | |
| RV026521 | 12 | 0 | 0% |
| (SEQ ID NO: 166) | | | |
| (CUCSA-WUS) | | | |
| RV026529 | 12 | 0 | 0% |
| (SEQ ID NO: 162) | | | |
| (AMBTR-WUS) | | | |
| RV026520 | 12 | 0 | 0% |
| (SEQ ID NO: 164) | | | |
| (POPTR-WUS) | | | |
| RV026592 | 12 | 0 | 0% |
| (SEQ ID NO: 196) | | | |
| (SL-WUS) | | | |
| RV026533 | 12 | 0 | 0% |
| (SEQ ID NO: 165) | | | |
| (AMAHY-WUS) | | | |
| RV022814 | 12 | 0 | 0% |
| (SEQ ID NO: 168) | | | |
| (NO WUS) | | | |
| (- Control) | | | |

*Efficacy of WUS genes from various species, as measured by the percentage of starting leaf section explants that produced green shoots from the section explants (on spectinomycin selection) at 50 days after Agrobacterium infection, wherein the starting leaf section explants were transformed by an Agrobacterium with a T-DNA containing a WUS gene as described above.

TABLE 9
Efficacy* of WUS genes from various species in Brassica leaf tissue

| | | | | Total |
|---|---|---|---|---|
| | Leaf Wounded | | | (Table 8 & 9) |
| | | Transgenic | Transgenic | Transgenic |
| Treatment | Leaf Sections | shoots | Shoots % | shoots % |
| RV026524 | 14 | 5 | 35.7% | 29% |
| (SEQ ID | | | | |
| NO: 158) | | | | |
| (MANES- | | | | |
| WUS) | | | | |
| RV026531 | 15 | 1 | 6.7% | 26% |
| (SEQ ID | | | | |
| NO: 157) | | | | |
| (MALDO- | | | | |
| WUS) | | | | |
| RV026528 | 12 | 3 | 25.0% | 25% |
| (SEQ ID | | | | |
| NO: 163) | | | | |
| (AQUCO- | | | | |
| WUS) | | | | |
| RV026522 | 17 | 0 | 0.0% | 21% |
| (SEQ ID | | | | |
| NO: 154) | | | | |
| (GNEGN- | | | | |
| WUS) | | | | |
| RV026526 | 12 | 0 | 0.0% | 21% |
| (SEQ ID | | | | |
| NO: 155) | | | | |
| (VITVI- | | | | |
| WUS) | | | | |
| RV026534 | 12 | 0 | 0.0% | 17% |
| (SEQ ID | | | | |
| NO: 156) | | | | |
| (PETHY- | | | | |
| WUS) | | | | |
| RV026530 | 12 | 3 | 25.0% | 13% |
| (SEQ ID | | | | |
| NO: 160) | | | | |
| (GOSHI- | | | | |
| WUS) | | | | |
| RV026527 | 12 | 2 | 16.7% | 8% |
| (SEQ ID | | | | |
| NO: 161) | | | | |
| (ZOSMA- | | | | |
| WUS) | | | | |
| RV026523 | 12 | 0 | 0.0% | 4% |
| (SEQ ID | | | | |
| NO: 159) | | | | |
| (KALFE- | | | | |
| WUS) | | | | |
| RV026525 | 11 | 0 | 0.0% | 0% |
| (SEQ ID | | | | |
| NO: 167) | | | | |
| (PINTA- | | | | |
| WUS) | | | | |
| RV026521 | 12 | 0 | 0.0% | 0% |
| (SEQ ID | | | | |
| NO: 166) | | | | |
| (CUCSA- | | | | |
| WUS) | | | | |
| RV026529 | 12 | 0 | 0.0% | 0% |
| (SEQ ID | | | | |
| NO: 162) | | | | |
| (AMBTR- | | | | |
| WUS) | | | | |
| RV026520 | 13 | 0 | 0.0% | 0% |
| (SEQ ID | | | | |
| NO: 164) | | | | |
| (POPTR- | | | | |
| WUS) | | | | |
| RV026592 | 11 | 0 | 0.0% | 0% |
| (SEQ ID | | | | |
| NO: 196) | | | | |
| (SL-WUS) | | | | |
| RV026533 | | | | |
| (SEQ ID | | | | |
| NO: 165) | 10 | 0 | 0.0% | 0% |
| (AMAHY- | | | | |
| WUS) | | | | |
| RV022814 | 12 | 0 | 0.0% | 0% |
| (SEQ ID | | | | |
| NO: 168) | | | | |
| (NO | | | | |
| WUS) | | | | |
| (−Control) | | | | |

*Efficacy of WUS genes from various species, as measured by the percentage of starting leaf section explants that produced green shoots from the section explants (on spectinomycin selection) at 50 days after Agrobacterium infection, wherein the starting leaf section explants were transformed by an Agrobacterium with a T-DNA containing a WUS gene as described above.

### Example 13: Orthologs of the Arabidopsis Wus Gene Stimulate Morphogenic Growth in Cowpea Leaves

WUS genes from twelve (12) different dicotyledonous species, two (2) gymnosperms, and one (1) monocot species were tested for efficacy by assessing their ability to stimulate growth of transgenic green shoot responses in cowpea leaves. For all treatments containing a WUS expression cassette, the configuration of the T-DNA was identical except for the WUS gene used in the construct. The T-DNA configuration was RB+CAMV35S PRO::WUS::OS-T28 TERM+GM-UBQ PRO::GM-UBQ SUTR::GM-UBQ INTRON1::ZS-YELLOW1 N1::NOS TERM+AT-UBIQ10 PRO::AT-UBIQ10 5UTR::AT-UBIQ10 INTRON1::CTP::SPCN::UBQ14 TERM+GM-EF1 A2 PRO::GM-EF1 A2 5′ UTR::GM-EF1 A2 INTRON1::DS-RED2::UBQ TERM+LB with the variable WUS gene in bold and italics.

Seeds of Vigna unguiculata IT86 D-1010 were sterilized with chlorine gas and germinated on solid medium containing MS basal salts and vitamins. The seedlings were grown at 28° C. in light and leaves were collected. The leaf tissue was transferred into 100×25 mm petri plates containing 10mls of 20 A medium (Table 10) with 200 mM acetosyringone and sliced into 3-5 mm long sections. After slicing, the 20 A solution was removed from each petri plate and replaced with 5 ml of the different Agrobacterium solutions (Agrobacterium strain LBA4404 THY-at an OD of 0.50 at 550 nM) containing expression cassettes with the different WUS genes described above. The plates were incubated at room temperature for 30-minutes. Then the treated leaf tissue was moved to 562V solid media (Table 10) for co-cultivation at 21° C. in the dark. After co-cultivation, the leaf explants were removed from the Agrobacterium solution, and lightly blotted onto sterile filter paper before placing onto 70 D media (Table 10) and moved to the light room (26° C. and bright light). After three days, tissue was placed onto 13113F resting media (Table 10). Tissue was imaged after 8 days on resting media. Transgenic regenerable plant structure (RPS) were tabulated for each infected leaf explant, see Table 11 below.

TABLE 10
Media

Infection medium (20A); MS salts and vitamins, 0.1 g/l myo-inositol,
0.5 g/l MES buffer, 0.1 mg/l a-NAA, 1 mg/l BAP, 10 μg/l gibberellic
acid, 50 mg/l thymidine, pH 5.5.
Co-cultivation medium (562V) 4g/L CHU(N6) basal salts, 1 ml/L1000x
Erikssons vitamins, 0.5 mg/L 49B-Thiamine, 30 g/L Sucrose, 2 mg/L
2A-2,4-D, 0.68 g/L L-Proline, 8 g/L Sigma Agar, pH 5.8
First culture medium (70D): 4.3 g/L MS salt mixture; 5 ml/L 36J-MS
vitamin stock solution; 0.1 g/L myo-inositol; 0.5 g/L MES buffer;
0.1 ml/L a-NAA (1.0 mg/ml); 1 ml/L BAP (1.0 mg/ml); 40 mg/L
adenine hemisulfate salt; 20 g/L sucrose; 0.5 g/L PVP40; 1.0 ml/L
silver nitrate (2.0 mg/ml); 10 ml/L carbenicillin (50.0 mg/ml); 5 g/L
TC agar; pH 5.7.
Resting media (13113F); 4.33 g/L MS salts and vitamins, 0.1 g/l
myo-inositol, 5 ml/L 36J MS Vitamin stock solution, 0.59 g/L MES
buffer, 30 g/l sucrose, 2 mg/L BAP, 0.5 mg/L Kinetin, 1 g/l PVP40,
1.5 g/L Meropenem, 6 g/l TC agar, pH 5.6.

As shown in Table 11, expressing WUS genes from phylogenetically divergent dicot, gymnosperm, or monocot species stimulated growth responses of transgenic regenerable plant structure (RPS) in cowpea leaves. Average responses ranged from 0.5 to 5.0 regenerable plant structure (RPS) per explant for WUS genes while negative controls of “NO WUS” and “No Agro” showed no transgenic regenerable plant structure (RPS).

TABLE 11

| | Average |
|---|---|
| WUS SPECIES | (RPS)/Leaf |
| RV026527 (SEQ ID NO: 161) (ZOSMA-WUS) | 5 |
| RV026592 (SEQ ID NO: 196) (SL-WUS) | 1.5 |
| RV026522 (SEQ ID NO: 154) (GNEGN-WUS) | 3 |
| RV026531 (SEQ ID NO: 157) (MALDO-WUS) | 3 |
| RV026524 (SEQ ID NO: 158) (MANES-WUS) | 5 |
| RV026526 (SEQ ID NO: 155) (VITVI-WUS) | 5 |
| RV026530 (SEQ ID NO: 160) (GOSHI-WUS) | 3 |
| RV026533 (SEQ ID NO: 165) (AMAHY-WUS) | 2 |
| RV026534 (SEQ ID NO: 156) (PETHY-WUS) | 3.5 |
| RV026529 (SEQ ID NO: 162) (AMBTR-WUS) | 1 |
| RV026520 (SEQ ID NO: 164) (POPTR-WUS) | 0.5 |
| RV026525 (SEQ ID NO: 167) (PINTA-WUS) | 0 |
| RV026521 (SEQ ID NO: 166) (CUCSA-WUS) | 1 |
| RV026523 (SEQ ID NO: 159) (KALFE-WUS) | 0 |
| RV022814 (SEQ ID NO: 168) (NO WUS) | 0 |
| (- Control) | |
| No Agro | 0 |

### Example 14: Wus Stimulates More Rapid Production of Green Shoots from Transformed Tobacco Leaf Segments

Tobacco (Nicotiana benthamiana) seedlings were germinated and grown under sterile light conditions on 90 A medium (Table 7). Leaves were excised and dissected into 2 cm×0.5 cm strips while submerged in 20 A liquid medium (Table 7). Each leaf yielded approximately seven leaf segments. Segments from four leaves were used per Agrobacterium infection. Agrobacterium strain LBA4404 THY-contained PHP71539 (a plasmid containing virulence genes disclosed in U.S. Pat. Pub. No. US20190078106, incorporated herein by reference in its entirety) and a binary plasmid harboring a T-DNA. The T-DNA for one treatment contained no WUS (NO WUS) expression cassette. For all treatments containing a WUS expression cassette, the configuration of the T-DNA was identical except for the WUS gene used in the construct. The T-DNA configuration was RB+CAMV35S PRO::WUS::OS-T28 TERM+GM-UBQ PRO::GM-UBQ SUTR::GM-UBQ INTRON1::ZS-YELLOW1 N1::NOS TERM+AT-UBIQ10 PRO::AT-UBIQ10 5UTR::AT-UBIQ10 INTRON1::CTP::SPCN::UBQ14 TERM+GM-EF1 A2 PRO::GM-EF1 A2 5′ UTR::GM-EF1 A2 INTRON1::DS-RED2::UBQ TERM+LB with the variable WUS gene in bold and italics. The Agrobacterium suspensions were prepared in 15 ml Falcon tubes in 20 A liquid medium to an OD₅₅₀ of 0.5. Twenty μl of the Agrobacterium suspension diluted into 10 ml of 20 A medium per well (in a multi-well plate) containing cut leaf tissues. The plates were shaken slowly for 10 minutes, then put under dim light (˜25-30 μE m⁻² s⁻¹) for a three-day co-cultivation with Agrobacterium in 20 A medium.

Following a three-day co-cultivation, leaf pieces were transferred onto 70 D medium (see Table 7) and cultured at 27° C. in the dark. Ten (10) days after transformation, tissues were sub-cultured onto new 70 D media. Eleven (11) days after transformation, incipient shoots (green regenerable plant structure) were counted on all the tissues, as well-defined shoots had not emerged yet, or were just beginning to emerge. Tables 12, 13 and 14 show the number of incipient shoots per tobacco leaf segment eleven (11) days after Agrobacterium infection. As shown in Tables 12, 13 and 14 the average number of incipient shoots observed per originally infected leaf segment increased (relative to the NO WUS control value of 2.2 incipient shoots/segment) for all the WUS genes tested, ranging from 4.2 for Manihot esculenta (RV026524 (SEQ ID NO: 158) (MANES-WUS)) up to 16.5 incipient shoots/segment for Kalanchoe fedtschenkoi (RV026523 (SEQ ID NO: 159) (KALFE-WUS)) and Petunia hybrid (RV026534 (SEQ ID NO: 156) (PETHY-WUS)).

TABLE 12

| | Name | | | | | |
|---|---|---|---|---|---|---|
| | NO WUS | VITVI-WUS | GOSHI-WUS | AQUCO-WUS | PINTA-WUS | AMAHY-WUS |
| | RV# | | | | | |
| | RV022814 | RV026526 | RV026530 | RV026528 | RV026525 | RV026533 |
| | (SEQ ID | (SEQ ID | (SEQ ID | (SEQ ID | (SEQ ID | (SEQ ID |
| | NO: 168) | NO: 155) | NO: 160) | NO: 163) | NO: 167) | NO: 165) |
| Incipient | 1 | 11 | 1 | 0 | 1 | 0 |
| shoots 11 days | 2 | 4 | 5 | 2 | 3 | 1 |
| after | 1 | 11 | 6 | 1 | 6 | 1 |
| transformation | 7 | 1 | 6 | 4 | 28 | 3 |
| | 1 | 4 | 4 | 5 | 11 | 3 |
| | 1 | 3 | 16 | 9 | 9 | 7 |
| | 3 | 4 | 16 | 4 | 8 | 4 |
| | 2 | 2 | 22 | 10 | 3 | 4 |
| | 2 | | | | 2 | 5 |
| | | | | | 16 | 17 |
| | | | | | 5 | |
| Ave # incipient | 2.2 | 5.0 | 9.5 | 4.4 | 8.4 | 4.5 |
| shoots/explant | | | | | | |
| St. dev. | 1.9 | 3.9 | 7.4 | 3.6 | 7.9 | 4.9 |
| incipient | | | | | | |
| shoots/explant | | | | | | |

TABLE 13

| | Name | | | | |
|---|---|---|---|---|---|
| | MANES- | ZOSMA- | SL- | MALDO- | KALFE- |
| | WUS | WUS | WUS | WUS | WUS |
| | RV # | | | | |
| | RV026524 | RV026527 | RV026592 | RV026531 | RV026523 |
| | (SEQ ID | (SEQ ID | (SEQ ID | (SEQ ID | (SEQ ID |
| | NO: 158) | NO: 161) | NO: 196) | NO: 157) | NO: 159) |
| Incipient | 0 | 16 | 12 | 11 | 23 |
| shoots | 0 | 6 | 14 | 6 | 32 |
| 11 days | 5 | 9 | 2 | 9 | 26 |
| after | 7 | 6 | 4 | 9 | 9 |
| transfor- | 9 | 5 | 10 | 3 | 17 |
| mation | 8 | 7 | 1 | 5 | 20 |
| | 1 | 4 | 10 | 5 | 13 |
| | 4 | 24 | 8 | 11 | 10 |
| | 3 | 4 | 11 | 19 | 6 |
| | 5 | 11 | 4 | 14 | 9 |
| | | 5 | 0 | | |
| | | 10 | 6 | | |
| Ave # | 4.2 | 8.9 | 6.8 | 9.2 | 16.5 |
| incipient | | | | | |
| shoots/ | | | | | |
| explant | | | | | |
| St .dev. | 3.2 | 5.9 | 4.6 | 4.8 | 8.6 |
| incipient | | | | | |
| shoots/ | | | | | |
| explant | | | | | |

TABLE 14

| | Name | | | | |
|---|---|---|---|---|---|
| | PETHY- | CUCSA- | AMBTR- | GNEGN- | POPTR- |
| | WUS | WUS | WUS | WUS | WUS |
| | RV # | | | | |
| | RV026534 | RV026521 | RV026529 | RV026522 | RV026520 |
| | (SEQ ID | (SEQ ID | (SEQ ID | (SEQ ID | (SEQ ID |
| | NO: 156) | NO: 166) | NO: 162) | NO: 154) | NO: 164) |
| Incipient | 19 | 5 | 0 | 2 | 14 |
| shoots | 12 | 7 | 15 | 3 | 16 |
| 11 days | 16 | 7 | 21 | 5 | 30 |
| after | 21 | 6 | 16 | 1 | 4 |
| transfor- | 9 | 8 | 16 | 6 | 3 |
| mation | 25 | 16 | 1 | 6 | 19 |
| | 15 | 25 | 10 | 1 | 25 |
| | 15 | | 1 | 20 | 15 |
| | | | 1 | 10 | 12 |
| | | | 5 | 3 | 12 |
| | | | 5 | 3 | 8 |
| | | | 16 | 0 | |
| | | | 11 | 2 | |
| | | | 25 | 4 | |
| | | | 20 | 6 | |
| | | | 8 | 4 | |
| Ave # | 16.5 | 10.6 | 10.7 | 4.8 | 14.4 |
| incipient | | | | | |
| shoots/ | | | | | |
| explant | | | | | |
| St .dev. | 5.1 | 7.3 | 8.0 | 4.8 | 8.2 |
| incipient | | | | | |
| shoots/ | | | | | |
| explant | | | | | |

Seventeen (17) days after Agrobacterium infection, the number of leaf segments that formed green shoots were counted and tabulated as shown in Tables 15, 16, and 177. As shown in Tables 15, 16, and 17, when the frequency of observing de novo green shoot formation on Agrobacterium-infected leaf segments was calculated as a percentage, approximately 11% of the “NO WUS” treatment leaf segments formed shoots. As shown in Tables 15, 16, and 17, frequencies for all the treatments in which a WUS expression cassette was present (for all the WUS orthologs tested) resulted in shoot formation frequencies above the control treatment (11%) and ranged from 25% for Vitus vinifera (grape) WUS) (RV026526 (SEQ ID NO: 155) (VITVI-WUS)) up to 100% for the Populus trichocarpa (poplar) WUS (RV026520 (SEQ ID NO: 164) (POPTR-WUS)). FIG. 1 is a graphical representation of the percent (%) of treated leaf segments that produced de novo green shoots.

TABLE 15

| | | VITVI-WUS | GOSHI-WUS | AQUCO-WUS | PINTA-WUS | AMAHY-WUS |
|---|---|---|---|---|---|---|
| | | RV026524 | RV026530 | RV026528 | RV026525 | RV026533 |
| 17 days after | | (SEQ ID | (SEQ ID | (SEQ ID | (SEQ ID | (SEQ ID |
| transformation | NO WUS | NO: 158) | NO: 160) | NO: 163) | NO: 167) | NO: 165) |
| leaf segments | 1 | 2 | 2 | 3 | 3 | 3 |
| with shoots | | | | | | |
| Total # leaf | 9 | 8 | 8 | 11 | 11 | 10 |
| segments | | | | | | |
| % leaf | 11.1% | 25.0% | 25.0% | 27.3% | 27.3% | 30.0% |
| segments with | | | | | | |
| shoots | | | | | | |

TABLE 16

| | MANES- | ZOSMA- | | MALDO- | KALFE- |
|---|---|---|---|---|---|
| 17 days | WUS | WUS | SL-WUS | WUS | WUS |
| after | RV026524 | RV026527 | RV026592 | RV026531 | RV026523 |
| transfor- | (SEQ ID | (SEQ ID | (SEQ ID | (SEQ ID | (SEQ ID |
| mation | NO: 158) | NO: 161) | NO: 196) | NO: 157) | NO: 159) |
| leaf | 4 | 6 | 7 | 6 | 6 |
| segments | | | | | |
| with | | | | | |
| shoots | | | | | |
| Total # | 10 | 12 | 12 | 10 | 10 |
| leaf | | | | | |
| segments | | | | | |
| % leaf | 40.0% | 50.0% | 58.3% | 60.0% | 60.0% |
| segments | | | | | |
| with | | | | | |
| shoots | | | | | |

TABLE 17

| | PETHY- | CUCSA- | AMBTR- | GNEGN- | POPTR- |
|---|---|---|---|---|---|
| 17 days | WUS | WUS | WUS | WUS | WUS |
| after | RV026534 | RV026521 | RV026529 | RV026522 | RV026520 |
| transfor- | (SEQ ID | (SEQ ID | (SEQ ID | (SEQ ID | (SEQ ID |
| mation | NO: 156) | NO: 166) | NO: 162) | NO: 154) | NO: 164) |
| leaf | 6 | 5 | 12 | 12 | 11 |
| segments | | | | | |
| with | | | | | |
| shoots | | | | | |
| Total # | 9 | 7 | 16 | 16 | 11 |
| leaf | | | | | |
| segments | | | | | |
| % leaf | 66.7% | 71.4% | 75.0% | 75.0% | 100.0% |
| segments | | | | | |
| with | | | | | |
| shoots | | | | | |

### Example 15: Canola Stem Transformation

Stem explants ranging from 0.3-0.8 cm in length were segmented and co-cultivated with LBA4404 THY-Agrobacterium containing the construct RV029481 ((SEQ ID NO: 191) (GG-WUS+HSP:CRE+IPT)). After two days of co-cultivation the explants were transferred to 70 A media and incubated at 26° C. and bright light (16 hr. photoperiod at 60-100 μE/m²/s). After 7 days or 14 days of incubation, plates containing the explants were heat shocked by transferring to 45° C. and 70% relative humidity for 2 hours, following which the plates were moved back to 26° C. The number of explants forming green somatic meristems were counted 18 days after co-cultivation (Table 18). Explants continued to form somatic meristems after the excision of the GG-WUS and IPT genes.

TABLE 18

| | | # of | # of | % of |
|---|---|---|---|---|
| | | explants | responsive | responsive |
| Construct | Treatment | infected | explants | explants |
| RV029481 | No Heat Shock | 24 | 15 | 62.5 |
| (SEQ ID | | | | |
| NO: 191) | | | | |
| (GG-WUS + | | | | |
| HSP:CRE + | | | | |
| IPT) | | | | |
| RV029481 | Heat Shock | 39 | 18 | 46.2 |
| (SEQ ID | after 7 d | | | |
| NO: 191) | | | | |
| (GG-WUS + | | | | |
| HSP:CRE + | | | | |
| IPT) | | | | |
| RV029481 | Heat Shock | 43 | 24 | 55.8 |
| (SEQ ID | after 14 d | | | |
| NO: 191) | | | | |
| (GG-WUS + | | | | |
| HSP:CRE + | | | | |
| IPT) | | | | |
| RV022814 | Control 26° C. | 24 | 0 | 0 |
| (SEQ ID | | | | |
| NO: 168) | | | | |
| (NO WUS) | | | | |
| (−Control) | | | | |

### Example 16: Use of Wus Orthologs from Four Different Species to Stimulate Direct Shoot Formation after Agrobacterium-Mediated Transformation of Soybean Leaf Segments

Agrobacterium strain LBA4404 THY-harboring a T-DNA with a WUS expression cassette, a heat-inducible CRE cassette, and SPCN expression cassette and a DS-RED2 expression cassette is used. The Agrobacterium strains contain the poplar WUS gene (RV029630 (SEQ ID NO: 193) (PT-WUS+HSP:CRE)), the poplar WUS gene fused to a nuclear localization sequence (RV029480 (SEQ ID NO: 194) (ALA-NLS-PT-WUS+HSP:CRE)), or a poplar WUS gene and an IPT gene (RV029479 (SEQ ID NO: 195) (PT-WUS+HSP:CRE+IPT)). Another set of Agrobacterium strains each contain the Gnetum WUS gene (RV029631 (SEQ ID NO: 192) (GG-WUS+HSP:CRE)), the Gnetum WUS gene fused to a nuclear localization sequence (RV029482 (SEQ ID NO: 190) (ALA-NLS-GG-WUS+HSP:CRE)), or a Gnetum WUS gene and an IPT gene (RV029481 (SEQ ID NO: 191) (GG-WUS+HSP:CRE+IPT)). The WUS, IPT and CRE genes are flanked by LOXP sites. The six Agrobacterium strains are used to transform segments of tissue cut from in vitro-grown, sterile, immature soybean leaves. Agrobacterium methods, transformation and media progression through co-cultivation and shoot elongation are as previously described.

Soybean lines such as elite lines, including, but not limited to, 93Y21 can be used in this process. Leaf and stem explants are harvested, and leaves are cut into 3-8 mm sections, while internodes (stem explants) are cut into sections of about 0.5 cm in length. Agrobacterium strain LBA4404 containing the vectors described above are used for the infections, and all bacterial cultures are adjusted to OD₅₅₀ of 0.5 for infection. All vectors contain the selectable marker gene SPCN (spectinomycin). Leaf and internode explants are infected for 30 min and are placed on co-cultivation medium for three days at 21° C. in dark. After co-cultivation, explants are transferred to shoot regeneration medium. Infection frequency is evaluated by screening the transient expression of the selectable marker gene at 5 and 20 days after transformation. Heat shock-driven expression of the CRE gene induces excision of any WUS, CRE, or IPT genes which were stably integrated into the genome. Shoot and root morphology are improved through excision of the IPT and WUS genes. Shoot regeneration is observed about 20 days after infection. In addition, transgenic shoots are evaluated for the presence of the marker gene.

For the control treatment (transforming with NO WUS or IPT in the T-DNA) which contained the SPCN and ZS-YELLOW1 N1 expression cassettes (RV022814 (SEQ ID NO: 168) (NO WUS)), it is expected that no direct shoot formation from leaf segments is observed. However, when transformations are performed which introduce T-DNAs containing the Gnetum and Poplar WUS genes, it is expected that green healthy shoots will be produced. Agrobacterium infection of leaf or stem tissue with all six WUS constructs is expected to produce healthy fertile plants in which the WUS, IPT and CRE genes have been excised.

### Example 17: Dicot Stem Transformation

Dicot stem explants ranging from 0.1-1.0 cm in length are segmented and co-cultivated with LBA4404 THY-Agrobacterium containing a construct comprising a nucleotide sequence encoding a functional WUS/WOX polypeptide, or a nucleotide sequence encoding a Babyboom (BBM) polypeptide or an Ovule Development Protein 2 (ODP2) polypeptide, or a combination of a nucleotide sequence encoding a functional WUS/WOX polypeptide and a nucleotide sequence encoding a Babyboom (BBM) polypeptide or an Ovule Development Protein 2 (ODP2) and the IPT gene and HSP:CRE. After two days of co-cultivation the explants are transferred to 70 A media and incubated at 26° C. and bright light (16 hr. photoperiod at 60-100 μE/m²/s). After 7 days or 14 days of incubation, plates containing the explants are heat shocked by transferring to 45° C. and 70% relative humidity for 2 hours, following which the plates are moved back to 26° C. It is expected that somatic meristems and/or somatic embryos are formed 10 days to 21 days after co-cultivation. It is expected that explants continue to form somatic meristems and/or somatic embryos after the excision of the WUS, BBM/ODP2 and IPT genes.

### Example 18: Dicot Leaf Transformation

Dicot leaf explants are cut into pieces of uniform size, approximately 10-80 millimeters and co-cultivated with LBA4404 THY-Agrobacterium containing a construct comprising a nucleotide sequence encoding a functional WUS/WOX polypeptide, or a nucleotide sequence encoding a Babyboom (BBM) polypeptide or an Ovule Development Protein 2 (ODP2) polypeptide, or a combination of a nucleotide sequence encoding a functional WUS/WOX polypeptide and a nucleotide sequence encoding a Babyboom (BBM) polypeptide or an Ovule Development Protein 2 (ODP2) and the IPT gene and HSP:CRE. After two days of co-cultivation the explants are transferred to 70 A media and incubated at 26° C. and bright light (16 hr. photoperiod at 60-100 μE/m²/s). After 7 days or 14 days of incubation, plates containing the explants are heat shocked by transferring to 45° C. and 70% relative humidity for 2 hours, following which the plates are moved back to 26° C. It is expected that somatic meristems and/or somatic embryos are formed 10 days to 21 days after co-cultivation. It is expected that explants continue to form somatic meristems and/or somatic embryos after the excision of the WUS, BBM/ODP2 and IPT genes.

### Example 19: Nuclease-Mediated Genome Modification of Dicot Leaf

Methods of making genome modifications mediated by CRISPR-Cas nucleases are described in U.S. Pat. No. 9,637,739, US2014/0068797, US2019/0040405, and U.S. Pat. No. 10,510,457, each of which is incorporated herein by reference in its entirety.

Dicot leaf explants are cut into pieces of uniform size, approximately 10-80 millimeters and co-cultivated with LBA4404 THY-Agrobacterium containing a construct comprising a nucleotide sequence encoding a functional WUS/WOX polypeptide, or a nucleotide sequence encoding a Babyboom (BBM) polypeptide or an Ovule Development Protein 2 (ODP2) polypeptide, or a combination of a nucleotide sequence encoding a functional WUS/WOX polypeptide and a nucleotide sequence encoding a Babyboom (BBM) polypeptide or an Ovule Development Protein 2 (ODP2) and providing a polynucleotide encoding a site-specific polypeptide or a site-specific polypeptide. After co-cultivation the leaf explants are transferred to fresh media and incubated at 26° C. and bright light (16 hr. photoperiod at 60-100 μE/m²/s). It is expected that a genome-edited regenerable plant structure containing the genome edit and no morphogenic gene expression cassette is formed 10 days to 21 days after co-cultivation. It is expected that the genome-edited regenerable plant structure containing the genome edit and no morphogenic gene expression cassette genome is formed at an increased frequency of from about 0.1% to about 1.0%, from about 1.1% to about 10%, from about 10.1% to about 20%, from about 20.1% to about 30%, from about 30.1% to about 40%, from about 40.1% to about 50%, from about 50.1% to about 60%, from about 60.1% to about 70%, from about 70.1% to about 80%, from about 80.1% to about 90%, and from about 90.1% to about 100%, compared to the frequency of genome-edited regenerable plant structures formed when the dicot vegetative plant organ or its composite tissue is not contacted with the morphogenic gene expression cassette.

### Example 20: Nuclease-Mediated Genome Modification of Dicot Stem

Methods of making genome modifications mediated by CRISPR-Cas nucleases are described in U.S. Pat. No. 9,637,739, US2014/0068797, US2019/0040405, and U.S. Pat. No. 10,510,457, each of which is incorporated herein by reference in its entirety.

Dicot stem explants ranging from 0.1-1.0 cm in length are segmented and co-cultivated with LBA4404 THY-Agrobacterium containing a construct comprising a nucleotide sequence encoding a functional WUS/WOX polypeptide, or a nucleotide sequence encoding a Babyboom (BBM) polypeptide or an Ovule Development Protein 2 (ODP2) polypeptide, or a combination of a nucleotide sequence encoding a functional WUS/WOX polypeptide and a nucleotide sequence encoding a Babyboom (BBM) polypeptide or an Ovule Development Protein 2 (ODP2) and providing a polynucleotide encoding a site-specific polypeptide or a site-specific polypeptide. After co-cultivation the stem explants are transferred to fresh media and incubated at 26° C. and bright light (16 hr. photoperiod at 60-100 μE/m²/s). It is expected that a genome-edited regenerable plant structure containing the genome edit and no morphogenic gene expression cassette is formed 10 days to 21 days after co-cultivation. It is expected that the genome-edited regenerable plant structure containing the genome edit and no morphogenic gene expression cassette genome is formed at an increased frequency of from about 0.1% to about 1.0%, from about 1.1% to about 10%, from about 10.1% to about 20%, from about 20.1% to about 30%, from about 30.1% to about 40%, from about 40.1% to about 50%, from about 50.1% to about 60%, from about 60.1% to about 70%, from about 70.1% to about 80%, from about 80.1% to about 90%, and from about 90.1% to about 100%, compared to the frequency of genome-edited regenerable plant structures formed when the dicot vegetative plant organ or its composite tissue is not contacted with the morphogenic gene expression cassette.

As used herein the singular forms “a”, “an”, and “the” include plural referents unless the context clearly dictates otherwise. Thus, for example, reference to “a cell” includes a plurality of such cells and reference to “the protein” includes reference to one or more proteins and equivalents thereof known to those skilled in the art, and so forth. All technical and scientific terms used herein have the same meaning as commonly understood to one of ordinary skill in the art to which this disclosure belongs unless clearly indicated otherwise.

All patents, publications and patent applications mentioned in the specification are indicative of the level of those skilled in the art to which this disclosure pertains. All patents, publications and patent applications are herein incorporated by reference in the entirety to the same extent as if each individual patent, publication or patent application was specifically and individually indicated to be incorporated by reference in its entirety.

Although the foregoing disclosure has been described in some detail by way of illustration and example for purposes of clarity of understanding, certain changes and modifications may be practiced within the scope of the appended claims.

1. A method of producing a transgenic dicot plant that contains a heterologous polynucleotide comprising:
contacting a dicot vegetative plant organ or its composite tissue with a T-DNA containing the heterologous polynucleotide and a morphogenic gene expression cassette;
selecting a plant cell containing the heterologous polynucleotide and no morphogenic gene expression cassette, wherein the plant cell forms a regenerable plant structure containing the heterologous polynucleotide and no morphogenic gene expression cassette; and
regenerating a transgenic plant from the regenerable plant structure containing the heterologous polynucleotide and no morphogenic gene expression cassette.

2. The method of claim 1, wherein the morphogenic gene expression cassette comprises:
(i) a nucleotide sequence encoding a functional WUS/WOX polypeptide; or
(ii) a nucleotide sequence encoding a Babyboom (BBM) polypeptide or an Ovule Development Protein 2 (ODP2) polypeptide; or
(iii) a combination of (i) and (ii).

3. The method of claim 2, wherein the nucleotide sequence encodes the functional WUS/WOX polypeptide.

4. The method of claim 3, wherein the nucleotide sequence encoding the functional WUS/WOX polypeptide is selected from WUS, WUS1, WUS2, WUS3, WOX2 A, WOX4, WOX5, and WOX9.

5. The method of claim 2, wherein the nucleotide sequence encodes the Babyboom (BBM) polypeptide or the Ovule Development Protein 2 (ODP2) polypeptide.

6. The method of claim 5, wherein the nucleotide sequence encoding the Babyboom (BBM) polypeptide is selected from BBM2, BMN2, and BMN3 or the Ovule Development Protein 2 (ODP2) polypeptide is ODP2.

7. The method of claim 2, wherein the nucleotide sequence encodes the functional WUS/WOX polypeptide and the Babyboom (BBM) polypeptide or the Ovule Development Protein 2 (ODP2) polypeptide.

8. The method of claim 7, wherein the nucleotide sequence encoding the functional WUS/WOX polypeptide is selected from WUS, WUS1, WUS2, WUS3, WOX2 A, WOX4, WOX5, and WOX9 and the Babyboom (BBM) polypeptide is selected from BBM2, BMN2, and BMN3 or the Ovule Development Protein 2 (ODP2) polypeptide is ODP2.

9. The method of claim 1, wherein the heterologous polynucleotide is selected from the group consisting of:
a heterologous polynucleotide conferring a nutritional enhancement, a heterologous polynucleotide conferring a modified oil content, a heterologous polynucleotide conferring a modified protein content, a heterologous polynucleotide conferring a modified metabolite content, a heterologous polynucleotide conferring increased yield, a heterologous polynucleotide conferring abiotic stress tolerance, a heterologous polynucleotide conferring drought tolerance, a heterologous polynucleotide conferring cold tolerance, a heterologous polynucleotide conferring herbicide tolerance, a heterologous polynucleotide conferring pest resistance, a heterologous polynucleotide conferring pathogen resistance, a heterologous polynucleotide conferring insect resistance, a heterologous polynucleotide conferring nitrogen use efficiency (NUE), a heterologous polynucleotide conferring disease resistance, a heterologous polynucleotide conferring increased biomass, a heterologous polynucleotide conferring an ability to alter a metabolic pathway, and a combination of the foregoing.

10. The method of claim 1, wherein the dicot vegetative plant organ or its composite tissue is selected from the group consisting of a leaf explant, a leaf primordia, a stipule, a cotyledon, a cotyledonary node, a mesocotyl, a stem explant, a primary root, a lateral secondary root, a root segment, a bud, and a meristem, including but not limited to an apical meristem, a root meristem, a secondary meristem, an axillary meristem, a floral meristem, and a combination of the foregoing.

11. The method of claim 10, wherein the leaf explant is selected from the group consisting of a leaf, a radical leaf, a cauline leaf, an alternate leaf, and opposite leaf, a decussate leaf, an opposite superposed leaf, a whorled leaf, a petiolate leaf, a sessile leaf, a subsessile leaf, a stipulate leaf, an exstipulate leaf, a simple leaf, a compound leaf, and a combination of the foregoing.

12. The method of claim 10, wherein the stem explant is selected from the group consisting of a stem nodal region, a stem internodal region, a petiole, a hypocotyl, an epicotyl, a stolon, a rhizome, a tuber, a corm, and a combination of the foregoing.

13. The method of claim 1, wherein the dicot is selected from the group consisting of soybean, cotton, sunflower, cassava, common bean, cowpea, tomato, potato, beet, grape, Eucalyptus, citrus, papaya, cacao, cucumber, apple, Capsicum, melon, and Brassica.

14. The method of claim 1, wherein the morphogenic gene expression cassette comprises a polynucleotide encoding a functional WUS/WOX polypeptide,
wherein the functional WUS/WOX polypeptide comprises an amino acid sequence of any of SEQ ID NOS: 61, 63, 65, 67, 69, 71, 73, 75, 77, 79, 81, 83, 85, 87, 89, 91, 93, 95, 97, 99, 101, 103, 105, 107, 128, 130, 132, 134, 136, 138, 140, 142, 144, 146, or 148; or wherein the functional WUS/WOX polypeptide is encoded by a nucleotide sequence of any of SEQ ID NOS: 60, 62, 64, 66, 68, 70, 72, 74, 76, 78, 80, 82, 84, 86, 88, 90, 92, 94, 96, 98, 100, 102, 104, 106, 127, 129, 131, 133, 135, 137, 139, 141, 143, 145, or 147.

15. The method of claim 1, wherein the morphogenic gene expression cassette further comprises a polynucleotide sequence encoding a site-specific recombinase selected from the group consisting of FLP, FLPe, KD, Cre, SSV1, lambda Int, phi C31 Int, HK022, R, B2, B3, Gin, Tn1721, CinH, ParA, Tn5053, Bxb1, TP907-1, or U153, wherein the site-specific recombinase is operably linked to a constitutive promoter, an inducible promoter, a tissue-specific promoter, or a developmentally regulated promoter.

16. The method of claim 15, further comprising excising the morphogenic gene expression cassette.

17. The transgenic plant produced by the method of claim 1.

18. A seed of the transgenic plant of claim 1, wherein the seed comprises the heterologous polynucleotide.

19. The method of claim 13, wherein the regenerable plant structure is formed at an increased frequency of from about 0.1% to about 1.0%, from about 1.1% to about 10%, from about 10.1% to about 20%, from about 20.1% to about 30%, from about 30.1% to about 40%, from about 40.1% to about 50%, from about 50.1% to about 60%, from about 60.1% to about 70%, from about 70.1% to about 80%, from about 80.1% to about 90%, and from about 90.1% to about 100%, compared to the frequency of regenerable plant structures formed when the dicot vegetative plant organ or its composite tissue is not contacted with the morphogenic gene expression cassette.

20. A method of producing a genome-edited dicot plant comprising:
contacting a dicot vegetative plant organ or its composite tissue with a T-DNA containing a morphogenic gene expression cassette and providing a polynucleotide encoding a site-specific polypeptide or a site-specific polypeptide;
selecting a plant cell containing a genome edit and no morphogenic gene expression cassette, wherein the plant cell forms a regenerable plant structure containing the genome edit and no morphogenic gene expression cassette; and
regenerating a genome-edited plant from the regenerable plant structure containing the genome edit and no morphogenic gene expression cassette.

21. The method of claim 20, wherein the morphogenic gene expression cassette comprises:
(i) a nucleotide sequence encoding a functional WUS/WOX polypeptide; or
(ii) a nucleotide sequence encoding a Babyboom (BBM) polypeptide or an Ovule Development Protein 2 (ODP2) polypeptide; or
(iii) a combination of (i) and (ii).

22. The method of claim 21, wherein the nucleotide sequence encodes the functional WUS/WOX polypeptide.

23. The method of claim 22, wherein the nucleotide sequence encoding the functional WUS/WOX polypeptide is selected from WUS, WUS1, WUS2, WUS3, WOX2 A, WOX4, WOX5, and WOX9.

24. The method of claim 21, wherein the nucleotide sequence encodes the Babyboom (BBM) polypeptide or the Ovule Development Protein 2 (ODP2) polypeptide.

25. The method of claim 24, wherein the nucleotide sequence encoding the Babyboom (BBM) polypeptide is selected from BBM2, BMN2, and BMN3 or the Ovule Development Protein 2 (ODP2) polypeptide is ODP2.

26. The method of claim 21, wherein the nucleotide sequence encodes the functional WUS/WOX polypeptide and the Babyboom (BBM) polypeptide or the Ovule Development Protein 2 (ODP2) polypeptide.

27. The method of claim 26, wherein the nucleotide sequence encoding the functional WUS/WOX polypeptide is selected from WUS, WUS1, WUS2, WUS3, WOX2 A, WOX4, WOX5, and WOX9 and the Babyboom (BBM) polypeptide is selected from BBM2, BMN2, and BMN3 or the Ovule Development Protein 2 (ODP2) polypeptide is ODP2.

28. The method of claim 21, wherein the site-specific polypeptide is selected from the group consisting of a zinc finger nuclease, a meganuclease, TALEN, and a CRISPR-Cas nuclease.

29. The method of claim 28, wherein the CRISPR-Cas nuclease is Cas9 or Cpfl nuclease and further comprising providing a guide RNA.

30. The method of claim 20, wherein the site-specific nuclease effects an insertion, a deletion, or a substitution mutation.

31. The method of claim 29, wherein the guide RNA and CRISPR-Cas nuclease is a ribonucleoprotein complex.

32. The method of claim 20, wherein the dicot vegetative plant organ or its composite tissue is selected from the group consisting of a leaf explant, a leaf primordia, a stipule, a cotyledon, a cotyledonary node, a mesocotyl, a stem explant, a primary root, a lateral secondary root, a root segment, a bud, and a meristem, including but not limited to an apical meristem, a root meristem, a secondary meristem, an axillary meristem, a floral meristem, and a combination of the foregoing.

33. The method of claim 32, wherein the leaf explant is selected from the group consisting of a leaf, a radical leaf, a cauline leaf, an alternate leaf, and opposite leaf, a decussate leaf, an opposite superposed leaf, a whorled leaf, a petiolate leaf, a sessile leaf, a subsessile leaf, a stipulate leaf, an exstipulate leaf, a simple leaf, a compound leaf, and a combination of the foregoing.

34. The method of claim 32, wherein the stem explant is selected from the group consisting of a stem nodal region, a stem internodal region, a petiole, a hypocotyl, an epicotyl, a stolon, a rhizome, a tuber, a corm, and a combination of the foregoing.

35. The method of claim 20, wherein the dicot, is selected from the group consisting of soybean, cotton, sunflower, cassava, common bean, cowpea, tomato, potato, beet, grape, Eucalyptus, citrus, papaya, cacao, cucumber, apple, Capsicum, melon, and Brassica.

36. The method of claim 20, wherein the morphogenic gene expression cassette comprises a polynucleotide encoding a functional WUS/WOX polypeptide,
wherein the functional WUS/WOX polypeptide comprises an amino acid sequence of any of SEQ ID NOS: 61, 63, 65, 67, 69, 71, 73, 75, 77, 79, 81, 83, 85, 87, 89, 91, 93, 95, 97, 99, 101, 103, 105, 107, 128, 130, 132, 134, 136, 138, 140, 142, 144, 146, or 148; or wherein the functional WUS/WOX polypeptide is encoded by a nucleotide sequence of any of SEQ ID NOS: 60, 62, 64, 66, 68, 70, 72, 74, 76, 78, 80, 82, 84, 86, 88, 90, 92, 94, 96, 98, 100, 102, 104, 106, 127, 129, 131, 133, 135, 137, 139, 141, 143, 145, or 147.

37. The method of claim 20 or 21, wherein the morphogenic gene expression cassette further comprises a polynucleotide sequence encoding a site-specific recombinase selected from the group consisting of FLP, FLPe, KD, Cre, SSV1, lambda Int, phi C31 Int, HK022, R, B2, B3, Gin, Tn1721, CinH, ParA, Tn5053, Bxb1, TP907-1, or U153, wherein the site-specific recombinase is operably linked to a constitutive promoter, an inducible promoter, a tissue-specific promoter, or a developmentally regulated promoter.

38. The method of claim 37, further comprising excising the morphogenic gene expression cassette.

39. The genome-edited plant produced by the method of claim 20.

40. A seed of the genome-edited plant of claim 20, wherein the seed comprises the genome edit.

41. The method of claim 35, wherein the regenerable plant structure is formed at an increased frequency of from about 0.1% to about 1.0%, from about 1.1% to about 10%, from about 10.1% to about 20%, from about 20.1% to about 30%, from about 30.1% to about 40%, from about 40.1% to about 50%, from about 50.1% to about 60%, from about 60.1% to about 70%, from about 70.1% to about 80%, from about 80.1% to about 90%, and from about 90.1% to about 100%, compared to the frequency of genome-edited regenerable plant structures formed when the dicot vegetative plant organ or its composite tissue is not contacted with the morphogenic gene expression cassette.

42. The method of claim 2, wherein the heterologous polynucleotide is selected from the group consisting of:
a heterologous polynucleotide conferring a nutritional enhancement, a heterologous polynucleotide conferring a modified oil content, a heterologous polynucleotide conferring a modified protein content, a heterologous polynucleotide conferring a modified metabolite content, a heterologous polynucleotide conferring increased yield, a heterologous polynucleotide conferring abiotic stress tolerance, a heterologous polynucleotide conferring drought tolerance, a heterologous polynucleotide conferring cold tolerance, a heterologous polynucleotide conferring herbicide tolerance, a heterologous polynucleotide conferring pest resistance, a heterologous polynucleotide conferring pathogen resistance, a heterologous polynucleotide conferring insect resistance, a heterologous polynucleotide conferring nitrogen use efficiency (NUE), a heterologous polynucleotide conferring disease resistance, a heterologous polynucleotide conferring increased biomass, a heterologous polynucleotide conferring an ability to alter a metabolic pathway, and a combination of the foregoing.

43. The method of claim 3, wherein the heterologous polynucleotide is selected from the group consisting of:
a heterologous polynucleotide conferring a nutritional enhancement, a heterologous polynucleotide conferring a modified oil content, a heterologous polynucleotide conferring a modified protein content, a heterologous polynucleotide conferring a modified metabolite content, a heterologous polynucleotide conferring increased yield, a heterologous polynucleotide conferring abiotic stress tolerance, a heterologous polynucleotide conferring drought tolerance, a heterologous polynucleotide conferring cold tolerance, a heterologous polynucleotide conferring herbicide tolerance, a heterologous polynucleotide conferring pest resistance, a heterologous polynucleotide conferring pathogen resistance, a heterologous polynucleotide conferring insect resistance, a heterologous polynucleotide conferring nitrogen use efficiency (NUE), a heterologous polynucleotide conferring disease resistance, a heterologous polynucleotide conferring increased biomass, a heterologous polynucleotide conferring an ability to alter a metabolic pathway, and a combination of the foregoing.

44. The method of claim 4, wherein the heterologous polynucleotide is selected from the group consisting of:
a heterologous polynucleotide conferring a nutritional enhancement, a heterologous polynucleotide conferring a modified oil content, a heterologous polynucleotide conferring a modified protein content, a heterologous polynucleotide conferring a modified metabolite content, a heterologous polynucleotide conferring increased yield, a heterologous polynucleotide conferring abiotic stress tolerance, a heterologous polynucleotide conferring drought tolerance, a heterologous polynucleotide conferring cold tolerance, a heterologous polynucleotide conferring herbicide tolerance, a heterologous polynucleotide conferring pest resistance, a heterologous polynucleotide conferring pathogen resistance, a heterologous polynucleotide conferring insect resistance, a heterologous polynucleotide conferring nitrogen use efficiency (NUE), a heterologous polynucleotide conferring disease resistance, a heterologous polynucleotide conferring increased biomass, a heterologous polynucleotide conferring an ability to alter a metabolic pathway, and a combination of the foregoing.

45. The method of claim 5, wherein the heterologous polynucleotide is selected from the group consisting of:
a heterologous polynucleotide conferring a nutritional enhancement, a heterologous polynucleotide conferring a modified oil content, a heterologous polynucleotide conferring a modified protein content, a heterologous polynucleotide conferring a modified metabolite content, a heterologous polynucleotide conferring increased yield, a heterologous polynucleotide conferring abiotic stress tolerance, a heterologous polynucleotide conferring drought tolerance, a heterologous polynucleotide conferring cold tolerance, a heterologous polynucleotide conferring herbicide tolerance, a heterologous polynucleotide conferring pest resistance, a heterologous polynucleotide conferring pathogen resistance, a heterologous polynucleotide conferring insect resistance, a heterologous polynucleotide conferring nitrogen use efficiency (NUE), a heterologous polynucleotide conferring disease resistance, a heterologous polynucleotide conferring increased biomass, a heterologous polynucleotide conferring an ability to alter a metabolic pathway, and a combination of the foregoing.

46. The method of claim 6, wherein the heterologous polynucleotide is selected from the group consisting of:
a heterologous polynucleotide conferring a nutritional enhancement, a heterologous polynucleotide conferring a modified oil content, a heterologous polynucleotide conferring a modified protein content, a heterologous polynucleotide conferring a modified metabolite content, a heterologous polynucleotide conferring increased yield, a heterologous polynucleotide conferring abiotic stress tolerance, a heterologous polynucleotide conferring drought tolerance, a heterologous polynucleotide conferring cold tolerance, a heterologous polynucleotide conferring herbicide tolerance, a heterologous polynucleotide conferring pest resistance, a heterologous polynucleotide conferring pathogen resistance, a heterologous polynucleotide conferring insect resistance, a heterologous polynucleotide conferring nitrogen use efficiency (NUE), a heterologous polynucleotide conferring disease resistance, a heterologous polynucleotide conferring increased biomass, a heterologous polynucleotide conferring an ability to alter a metabolic pathway, and a combination of the foregoing.

47. The method of claim 7, wherein the heterologous polynucleotide is selected from the group consisting of:
a heterologous polynucleotide conferring a nutritional enhancement, a heterologous polynucleotide conferring a modified oil content, a heterologous polynucleotide conferring a modified protein content, a heterologous polynucleotide conferring a modified metabolite content, a heterologous polynucleotide conferring increased yield, a heterologous polynucleotide conferring abiotic stress tolerance, a heterologous polynucleotide conferring drought tolerance, a heterologous polynucleotide conferring cold tolerance, a heterologous polynucleotide conferring herbicide tolerance, a heterologous polynucleotide conferring pest resistance, a heterologous polynucleotide conferring pathogen resistance, a heterologous polynucleotide conferring insect resistance, a heterologous polynucleotide conferring nitrogen use efficiency (NUE), a heterologous polynucleotide conferring disease resistance, a heterologous polynucleotide conferring increased biomass, a heterologous polynucleotide conferring an ability to alter a metabolic pathway, and a combination of the foregoing.

48. The method of claim 8, wherein the heterologous polynucleotide is selected from the group consisting of:
a heterologous polynucleotide conferring a nutritional enhancement, a heterologous polynucleotide conferring a modified oil content, a heterologous polynucleotide conferring a modified protein content, a heterologous polynucleotide conferring a modified metabolite content, a heterologous polynucleotide conferring increased yield, a heterologous polynucleotide conferring abiotic stress tolerance, a heterologous polynucleotide conferring drought tolerance, a heterologous polynucleotide conferring cold tolerance, a heterologous polynucleotide conferring herbicide tolerance, a heterologous polynucleotide conferring pest resistance, a heterologous polynucleotide conferring pathogen resistance, a heterologous polynucleotide conferring insect resistance, a heterologous polynucleotide conferring nitrogen use efficiency (NUE), a heterologous polynucleotide conferring disease resistance, a heterologous polynucleotide conferring increased biomass, a heterologous polynucleotide conferring an ability to alter a metabolic pathway, and a combination of the foregoing.

49. The method of claim 10, wherein the dicot is selected from the group consisting of soybean, cotton, sunflower, cassava, common bean, cowpea, tomato, potato, beet, grape, Eucalyptus, citrus, papaya, cacao, cucumber, apple, Capsicum, melon, and Brassica.

50. The method of claim 2, wherein the morphogenic gene expression cassette comprises a polynucleotide encoding a functional WUS/WOX polypeptide,
wherein the functional WUS/WOX polypeptide comprises an amino acid sequence of any of SEQ ID NOS: 61, 63, 65, 67, 69, 71, 73, 75, 77, 79, 81, 83, 85, 87, 89, 91, 93, 95, 97, 99, 101, 103, 105, 107, 128, 130, 132, 134, 136, 138, 140, 142, 144, 146, or 148; or wherein the functional WUS/WOX polypeptide is encoded by a nucleotide sequence of any of SEQ ID NOS: 60, 62, 64, 66, 68, 70, 72, 74, 76, 78, 80, 82, 84, 86, 88, 90, 92, 94, 96, 98, 100, 102, 104, 106, 127, 129, 131, 133, 135, 137, 139, 141, 143, 145, or 147.

51. The method of claim 3, wherein the morphogenic gene expression cassette comprises a polynucleotide encoding a functional WUS/WOX polypeptide,
wherein the functional WUS/WOX polypeptide comprises an amino acid sequence of any of SEQ ID NOS: 61, 63, 65, 67, 69, 71, 73, 75, 77, 79, 81, 83, 85, 87, 89, 91, 93, 95, 97, 99, 101, 103, 105, 107, 128, 130, 132, 134, 136, 138, 140, 142, 144, 146, or 148; or wherein the functional WUS/WOX polypeptide is encoded by a nucleotide sequence of any of SEQ ID NOS: 60, 62, 64, 66, 68, 70, 72, 74, 76, 78, 80, 82, 84, 86, 88, 90, 92, 94, 96, 98, 100, 102, 104, 106, 127, 129, 131, 133, 135, 137, 139, 141, 143, 145, or 147.

52. The method of claim 4, wherein the morphogenic gene expression cassette comprises a polynucleotide encoding a functional WUS/WOX polypeptide,
wherein the functional WUS/WOX polypeptide comprises an amino acid sequence of any of SEQ ID NOS: 61, 63, 65, 67, 69, 71, 73, 75, 77, 79, 81, 83, 85, 87, 89, 91, 93, 95, 97, 99, 101, 103, 105, 107, 128, 130, 132, 134, 136, 138, 140, 142, 144, 146, or 148; or wherein the functional WUS/WOX polypeptide is encoded by a nucleotide sequence of any of SEQ ID NOS: 60, 62, 64, 66, 68, 70, 72, 74, 76, 78, 80, 82, 84, 86, 88, 90, 92, 94, 96, 98, 100, 102, 104, 106, 127, 129, 131, 133, 135, 137, 139, 141, 143, 145, or 147.

53. The method of claim 7, wherein the morphogenic gene expression cassette comprises a polynucleotide encoding a functional WUS/WOX polypeptide,
wherein the functional WUS/WOX polypeptide comprises an amino acid sequence of any of SEQ ID NOS: 61, 63, 65, 67, 69, 71, 73, 75, 77, 79, 81, 83, 85, 87, 89, 91, 93, 95, 97, 99, 101, 103, 105, 107, 128, 130, 132, 134, 136, 138, 140, 142, 144, 146, or 148; or wherein the functional WUS/WOX polypeptide is encoded by a nucleotide sequence of any of SEQ ID NOS: 60, 62, 64, 66, 68, 70, 72, 74, 76, 78, 80, 82, 84, 86, 88, 90, 92, 94, 96, 98, 100, 102, 104, 106, 127, 129, 131, 133, 135, 137, 139, 141, 143, 145, or 147.

54. The method of claim 8, wherein the morphogenic gene expression cassette comprises a polynucleotide encoding a functional WUS/WOX polypeptide,
wherein the functional WUS/WOX polypeptide comprises an amino acid sequence of any of SEQ ID NOS: 61, 63, 65, 67, 69, 71, 73, 75, 77, 79, 81, 83, 85, 87, 89, 91, 93, 95, 97, 99, 101, 103, 105, 107, 128, 130, 132, 134, 136, 138, 140, 142, 144, 146, or 148; or wherein the functional WUS/WOX polypeptide is encoded by a nucleotide sequence of any of SEQ ID NOS: 60, 62, 64, 66, 68, 70, 72, 74, 76, 78, 80, 82, 84, 86, 88, 90, 92, 94, 96, 98, 100, 102, 104, 106, 127, 129, 131, 133, 135, 137, 139, 141, 143, 145, or 147.

55. The method of claim 2, wherein the morphogenic gene expression cassette further comprises a polynucleotide sequence encoding a site-specific recombinase selected from the group consisting of FLP, FLPe, KD, Cre, SSV1, lambda Int, phi C31 Int, HK022, R, B2, B3, Gin, Tn1721, CinH, ParA, Tn5053, Bxb1, TP907-1, or U153, wherein the site-specific recombinase is operably linked to a constitutive promoter, an inducible promoter, a tissue-specific promoter, or a developmentally regulated promoter.

56. The transgenic plant produced by the method of claim 16.

57. A seed of the transgenic plant of claim 17, wherein the seed comprises the heterologous polynucleotide.

58. The method of claim 14, wherein the regenerable plant structure is formed at an increased frequency of from about 0.1% to about 1.0%, from about 1.1% to about 10%, from about 10.1% to about 20%, from about 20.1% to about 30%, from about 30.1% to about 40%, from about 40.1% to about 50%, from about 50.1% to about 60%, from about 60.1% to about 70%, from about 70.1% to about 80%, from about 80.1% to about 90%, and from about 90.1% to about 100%, compared to the frequency of regenerable plant structures formed when the dicot vegetative plant organ or its composite tissue is not contacted with the morphogenic gene expression cassette.

59. The method of claim 28, wherein the site-specific nuclease effects an insertion, a deletion, or a substitution mutation.

60. The method of claim 29, wherein the site-specific nuclease effects an insertion, a deletion, or a substitution mutation.

61. The method of claim 32, wherein the dicot, is selected from the group consisting of soybean, cotton, sunflower, cassava, common bean, cowpea, tomato, potato, beet, grape, Eucalyptus, citrus, papaya, cacao, cucumber, apple, Capsicum, melon, and Brassica.

62. The method of claim 21, wherein the morphogenic gene expression cassette comprises a polynucleotide encoding a functional WUS/WOX polypeptide,
wherein the functional WUS/WOX polypeptide comprises an amino acid sequence of any of SEQ ID NOS: 61, 63, 65, 67, 69, 71, 73, 75, 77, 79, 81, 83, 85, 87, 89, 91, 93, 95, 97, 99, 101, 103, 105, 107, 128, 130, 132, 134, 136, 138, 140, 142, 144, 146, or 148; or wherein the functional WUS/WOX polypeptide is encoded by a nucleotide sequence of any of SEQ ID NOS: 60, 62, 64, 66, 68, 70, 72, 74, 76, 78, 80, 82, 84, 86, 88, 90, 92, 94, 96, 98, 100, 102, 104, 106, 127, 129, 131, 133, 135, 137, 139, 141, 143, 145, or 147.

63. The method of claim 22, wherein the morphogenic gene expression cassette comprises a polynucleotide encoding a functional WUS/WOX polypeptide,
wherein the functional WUS/WOX polypeptide comprises an amino acid sequence of any of SEQ ID NOS: 61, 63, 65, 67, 69, 71, 73, 75, 77, 79, 81, 83, 85, 87, 89, 91, 93, 95, 97, 99, 101, 103, 105, 107, 128, 130, 132, 134, 136, 138, 140, 142, 144, 146, or 148; or wherein the functional WUS/WOX polypeptide is encoded by a nucleotide sequence of any of SEQ ID NOS: 60, 62, 64, 66, 68, 70, 72, 74, 76, 78, 80, 82, 84, 86, 88, 90, 92, 94, 96, 98, 100, 102, 104, 106, 127, 129, 131, 133, 135, 137, 139, 141, 143, 145, or 147.

64. The method of claim 23, wherein the morphogenic gene expression cassette comprises a polynucleotide encoding a functional WUS/WOX polypeptide,
wherein the functional WUS/WOX polypeptide comprises an amino acid sequence of any of SEQ ID NOS: 61, 63, 65, 67, 69, 71, 73, 75, 77, 79, 81, 83, 85, 87, 89, 91, 93, 95, 97, 99, 101, 103, 105, 107, 128, 130, 132, 134, 136, 138, 140, 142, 144, 146, or 148; or wherein the functional WUS/WOX polypeptide is encoded by a nucleotide sequence of any of SEQ ID NOS: 60, 62, 64, 66, 68, 70, 72, 74, 76, 78, 80, 82, 84, 86, 88, 90, 92, 94, 96, 98, 100, 102, 104, 106, 127, 129, 131, 133, 135, 137, 139, 141, 143, 145, or 147.

65. The method of claim 26, wherein the morphogenic gene expression cassette comprises a polynucleotide encoding a functional WUS/WOX polypeptide,
wherein the functional WUS/WOX polypeptide comprises an amino acid sequence of any of SEQ ID NOS: 61, 63, 65, 67, 69, 71, 73, 75, 77, 79, 81, 83, 85, 87, 89, 91, 93, 95, 97, 99, 101, 103, 105, 107, 128, 130, 132, 134, 136, 138, 140, 142, 144, 146, or 148; or wherein the functional WUS/WOX polypeptide is encoded by a nucleotide sequence of any of SEQ ID NOS: 60, 62, 64, 66, 68, 70, 72, 74, 76, 78, 80, 82, 84, 86, 88, 90, 92, 94, 96, 98, 100, 102, 104, 106, 127, 129, 131, 133, 135, 137, 139, 141, 143, 145, or 147.

66. The method of claim 27, wherein the morphogenic gene expression cassette comprises a polynucleotide encoding a functional WUS/WOX polypeptide,
wherein the functional WUS/WOX polypeptide comprises an amino acid sequence of any of SEQ ID NOS: 61, 63, 65, 67, 69, 71, 73, 75, 77, 79, 81, 83, 85, 87, 89, 91, 93, 95, 97, 99, 101, 103, 105, 107, 128, 130, 132, 134, 136, 138, 140, 142, 144, 146, or 148; or wherein the functional WUS/WOX polypeptide is encoded by a nucleotide sequence of any of SEQ ID NOS: 60, 62, 64, 66, 68, 70, 72, 74, 76, 78, 80, 82, 84, 86, 88, 90, 92, 94, 96, 98, 100, 102, 104, 106, 127, 129, 131, 133, 135, 137, 139, 141, 143, 145, or 147.

67. The method of claim 21, wherein the morphogenic gene expression cassette further comprises a polynucleotide sequence encoding a site-specific recombinase selected from the group consisting of FLP, FLPe, KD, Cre, SSV1, lambda Int, phi C31 Int, HK022, R, B2, B3, Gin, Tn1721, CinH, ParA, Tn5053, Bxb1, TP907-1, or U153, wherein the site-specific recombinase is operably linked to a constitutive promoter, an inducible promoter, a tissue-specific promoter, or a developmentally regulated promoter.

68. The genome-edited plant produced by the method of claim 38.

69. A seed of the genome-edited plant of claim 39, wherein the seed comprises the genome edit.

70. The method of claim 36, wherein the regenerable plant structure is formed at an increased frequency of from about 0.1% to about 1.0%, from about 1.1% to about 10%, from about 10.1% to about 20%, from about 20.1% to about 30%, from about 30.1% to about 40%, from about 40.1% to about 50%, from about 50.1% to about 60%, from about 60.1% to about 70%, from about 70.1% to about 80%, from about 80.1% to about 90%, and from about 90.1% to about 100%, compared to the frequency of genome-edited regenerable plant structures formed when the dicot vegetative plant organ or its composite tissue is not contacted with the morphogenic gene expression cassette.
